SEQUENCE LISTING (772 sequences)

SEQ ID NO: 1 - RNA, 19 nt, Artificial Sequence
  Description of Artificial Sequence Synthetic
uccuaggaccccugcucgu

SEQ ID NO: 2 - RNA, 19 nt, Artificial Sequence
  Description of Artificial Sequence Synthetic
gagucuagacucguggugg

SEQ ID NO: 3 - RNA, 19 nt, Artificial Sequence
  Description of Artificial Sequence Synthetic
gugcacuucgcuucaccuc

SEQ ID NO: 4 - RNA, 19 nt, Artificial Sequence
  Description of Artificial Sequence Synthetic
gccauggcguuaguaugag

SEQ ID NO: 5 - RNA, 19 nt, Artificial Sequence
  Description of Artificial Sequence Synthetic
cucccgggagagccauagu

SEQ ID NO: 6 - RNA, 19 nt, Artificial Sequence
  Description of Artificial Sequence Synthetic
accggugaguacaccggaa

SEQ ID NO: 7 - RNA, 19 nt, Artificial Sequence
  Description of Artificial Sequence Synthetic
cccgggaggucucguagac

SEQ ID NO: 8 - RNA, 19 nt, Artificial Sequence
  Description of Artificial Sequence Synthetic
uuuaaaaggcacccagcac

SEQ ID NO: 9 - RNA, 19 nt, Artificial Sequence
  Description of Artificial Sequence Synthetic
auauauaugauaaagcauu

SEQ ID NO: 10 - RNA, 27 nt, Artificial Sequence
  Description of Artificial Sequence Synthetic
aaugcuuuaucauauauaugauaaagc

SEQ ID NO: 11 - RNA, 25 nt, Artificial Sequence
  Description of Artificial Sequence Synthetic
aaugcuuuaucauauaugauaaagc

SEQ ID NO: 12 - RNA, 23 nt, Artificial Sequence
  Description of Artificial Sequence Synthetic
aaugcuuuaucauaugauaaagc

SEQ ID NO: 13 - RNA, 24 nt, Artificial Sequence
  Description of Artificial Sequence Synthetic
aaugcuuuaucauaugauaaagca

SEQ ID NO: 14 - RNA, 28 nt, Artificial Sequence
  Description of Artificial Sequence Synthetic
aaugcuuuaucauauaugauaaagcauu

SEQ ID NO: 15 - DNA, 21 bp, Artificial Sequence
  Description of Artificial Sequence Synthetic
gaacugaguuuaaaaggcatt

SEQ ID NO: 16 - DNA, 21 bp, Artificial Sequence
  Description of Artificial Sequence Synthetic
ugccuuuuaaacucaguuctt

SEQ ID NO: 17 - DNA, 21 bp, Artificial Sequence
  Description of Artificial Sequence Synthetic
acggaaaauuugagucaagtt

SEQ ID NO: 18 - DNA, 21 bp, Artificial Sequence
  Description of Artificial Sequence Synthetic
cuugacucaaauuuuccgutt

SEQ ID NO: 19 - DNA, 21 bp, Artificial Sequence
  Description of Artificial Sequence Synthetic
gcgagggaguucuucuucutt

SEQ ID NO: 20 - DNA, 21 bp, Artificial Sequence
  Description of Artificial Sequence Synthetic
ugccuuuuaaacucaguuctt

SEQ ID NO: 21 - DNA, 21 bp, Artificial Sequence
  Description of Artificial Sequence Synthetic
acggaaaauuugagucaagnt

SEQ ID NO: 22 - DNA, 21 bp, Artificial Sequence
  Description of Artificial Sequence Synthetic
cuugacucaaauuuuccgutt

SEQ ID NO: 23 - DNA, 21 bp, Artificial Sequence
  Description of Artificial Sequence Synthetic
cugaguuuaaaaggcacccnt

SEQ ID NO: 24 - DNA, 21 bp, Artificial Sequence
  Description of Artificial Sequence Synthetic
gggugccuuuuaaacucagtt

SEQ ID NO: 25 - DNA, 21 bp, Artificial Sequence
  Description of Artificial Sequence Synthetic
cccacggaaaauuugagucnt

SEQ ID NO: 26 - DNA, 21 bp, Artificial Sequence
  Description of Artificial Sequence Synthetic
gacucaaauuuuccgugggtt

SEQ ID NO: 27 - DNA, 21 bp, Artificial Sequence
  Description of Artificial Sequence Synthetic
ugugcacuucgcuucaccunt

SEQ ID NO: 28 - DNA, 21 bp, Artificial Sequence
  Description of Artificial Sequence Synthetic
aggugaagcgaagugcacatt

SEQ ID NO: 29 - DNA, 21 bp, Artificial Sequence
  Description of Artificial Sequence Synthetic
uccacuucgcuucacgugunt

SEQ ID NO: 30 - DNA, 21 bp, Artificial Sequence
  Description of Artificial Sequence Synthetic
acacgugaagcgaaguggatt

SEQ ID NO: 31 - RNA, 30 nt, Artificial Sequence
  Description of Artificial Sequence Synthetic
aggugaagcgaagugcacacuucgcuucau

SEQ ID NO: 32 - RNA, 29 nt, Artificial Sequence
  Description of Artificial Sequence Synthetic
ugcugggugccuuuuaaaaggcacccagc

SEQ ID NO: 33 - RNA, 28 nt, Artificial Sequence
  Description of Artificial Sequence Synthetic
gcugggugccuuuuaaaaggcacccagc

SEQ ID NO: 34 - RNA, 28 nt, Artificial Sequence
  Description of Artificial Sequence Synthetic
gcugggugccuuuuaaaaggcacccagc

SEQ ID NO: 35 - RNA, 27 nt, Artificial Sequence
  Description of Artificial Sequence Synthetic
gcugggugccuuuuaaaaggcacccag

SEQ ID NO: 36 - RNA, 26 nt, Artificial Sequence
  Description of Artificial Sequence Synthetic
cugggugccuuuuaaaaggcacccag

SEQ ID NO: 37 - RNA, 25 nt, Artificial Sequence
  Description of Artificial Sequence Synthetic
cugggugccuuuuaaaaggcaccca

SEQ ID NO: 38 - RNA, 24 nt, Artificial Sequence
  Description of Artificial Sequence Synthetic
ugggugccuuuuaaaaggcaccca

SEQ ID NO: 39 - RNA, 24 nt, Artificial Sequence
  Description of Artificial Sequence Synthetic
ugggugccuuuuaaaaggcaccca

SEQ ID NO: 40 - DNA, 26 bp, Artificial Sequence
  Description of Artificial Sequence Synthetic
ugggugccuuuuaaaaggcacccatt

SEQ ID NO: 41 - RNA, 26 nt, Artificial Sequence
  Description of Artificial Sequence Synthetic
ugcuuuaucauauauaugauaaagca

SEQ ID NO: 42 - RNA, 25 nt, Artificial Sequence
  Description of Artificial Sequence Synthetic
ugcuuuaucauauauaugauaaagc

SEQ ID NO: 43 - RNA, 24 nt, Artificial Sequence
  Description of Artificial Sequence Synthetic
gcuuuaucauauauaugauaaagc

SEQ ID NO: 44 - RNA, 28 nt, Artificial Sequence
  Description of Artificial Sequence Synthetic
aaugcuuuaucauauaugauaaagcauu

SEQ ID NO: 45 - RNA, 28 nt, Artificial Sequence
  Description of Artificial Sequence Synthetic
aaugcuuuaucauauaugauaaagcauu

SEQ ID NO: 46 - DNA, 30 bp, Artificial Sequence
  Description of Artificial Sequence Synthetic
aaugcuuuaucauauaugauaaagcauutt

SEQ ID NO: 47 - RNA, 27 nt, Artificial Sequence
  Description of Artificial Sequence Synthetic
aaugcuuuaucauauaugauaaagcau

SEQ ID NO: 48 - RNA, 26 nt, Artificial Sequence
  Description of Artificial Sequence Synthetic
augcuuuaucauauaugauaaagcau

SEQ ID NO: 49 - DNA, 27 bp, Artificial Sequence
  Description of Artificial Sequence Synthetic
augcuuuaucauauaugauaaagcaut

SEQ ID NO: 50 - DNA, 28 bp, Artificial Sequence
  Description of Artificial Sequence Synthetic
augcuuuaucauauaugauaaagcautt

SEQ ID NO: 51 - RNA, 25 nt, Artificial Sequence
  Description of Artificial Sequence Synthetic
augcuuuaucauauaugauaaagca

SEQ ID NO: 52 - RNA, 28 nt, Artificial Sequence
  Description of Artificial Sequence Synthetic
aaugcuuuaucauauaucuauaagcauu

SEQ ID NO: 53 - RNA, 28 nt, Artificial Sequence
  Description of Artificial Sequence Synthetic
aaugcuuuuaguuauaugauaaagcauu

SEQ ID NO: 54 - RNA, 28 nt, Artificial Sequence
  Description of Artificial Sequence Synthetic
aauccuuaaucuuauuugauaaagcauu

SEQ ID NO: 55 - RNA, 28 nt, Artificial Sequence
  Description of Artificial Sequence Synthetic
aaugcuuuaucauauaugauaaagcauu

SEQ ID NO: 56 - RNA, 28 nt, Artificial Sequence
  Description of Artificial Sequence Synthetic
aaugcuuuaucauauaugauaaagcauu

SEQ ID NO: 57 - RNA, 28 nt, Artificial Sequence
  Description of Artificial Sequence Synthetic
cgagcagggguccuaggaccccugcucg

SEQ ID NO: 58 - RNA, 26 nt, Artificial Sequence
  Description of Artificial Sequence Synthetic
gagcagggguccuaggaccccugcuc

SEQ ID NO: 59 - RNA, 24 nt, Artificial Sequence
  Description of Artificial Sequence Synthetic
agcagggguccuaggaccccugcu

SEQ ID NO: 60 - RNA, 22 nt, Artificial Sequence
  Description of Artificial Sequence Synthetic
gcagggguccuaggaccccugc

SEQ ID NO: 61 - RNA, 26 nt, Artificial Sequence
  Description of Artificial Sequence Synthetic
ccaccacgagucuagacucguggugg

SEQ ID NO: 62 - RNA, 22 nt, Artificial Sequence
  Description of Artificial Sequence Synthetic
accacgagucuagacucguggu

SEQ ID NO: 63 - RNA, 20 nt, Artificial Sequence
  Description of Artificial Sequence Synthetic
ccacgagucuagacucgugg

SEQ ID NO: 64 - RNA, 18 nt, Artificial Sequence
  Description of Artificial Sequence Synthetic
cacgagucuagacucgug

SEQ ID NO: 65 - RNA, 28 nt, Artificial Sequence
  Description of Artificial Sequence Synthetic
ggugaagcgaagugcacuucgcuucacc

SEQ ID NO: 66 - RNA, 26 nt, Artificial Sequence
  Description of Artificial Sequence Synthetic
gugaagcgaagugcacuucgcuucac

SEQ ID NO: 67 - RNA, 24 nt, Artificial Sequence
  Description of Artificial Sequence Synthetic
ugaagcgaagugcacuucgcuuca

SEQ ID NO: 68 - RNA, 28 nt, Artificial Sequence
  Description of Artificial Sequence Synthetic
ucauacuaacgccauggcguuaguauga

SEQ ID NO: 69 - RNA, 26 nt, Artificial Sequence
  Description of Artificial Sequence Synthetic
cauacuaacgccauggcguuaguaug

SEQ ID NO: 70 - RNA, 24 nt, Artificial Sequence
  Description of Artificial Sequence Synthetic
auacuaacgccauggcguuaguau

SEQ ID NO: 71 - RNA, 22 nt, Artificial Sequence
  Description of Artificial Sequence Synthetic
uacuaacgccauggcguuagua

SEQ ID NO: 72 - RNA, 28 nt, Artificial Sequence
  Description of Artificial Sequence Synthetic
acuauggcucucccgggagagccauagu

SEQ ID NO: 73 - RNA, 26 nt, Artificial Sequence
  Description of Artificial Sequence Synthetic
cuauggcucucccgggagagccauag

SEQ ID NO: 74 - RNA, 24 nt, Artificial Sequence
  Description of Artificial Sequence Synthetic
uauggcucucccgggagagccaua

SEQ ID NO: 75 - RNA, 22 nt, Artificial Sequence
  Description of Artificial Sequence Synthetic
auggcucucccgggagagccau

SEQ ID NO: 76 - RNA, 20 nt, Artificial Sequence
  Description of Artificial Sequence Synthetic
uggcucucccgggagagcca

SEQ ID NO: 77 - RNA, 28 nt, Artificial Sequence
  Description of Artificial Sequence Synthetic
ccgguguacucaccggugaguacaccgg

SEQ ID NO: 78 - RNA, 26 nt, Artificial Sequence
  Description of Artificial Sequence Synthetic
cgguguacucaccggugaguacaccg

SEQ ID NO: 79 - RNA, 24 nt, Artificial Sequence
  Description of Artificial Sequence Synthetic
gguguacucaccggugaguacacc

SEQ ID NO: 80 - RNA, 28 nt, Artificial Sequence
  Description of Artificial Sequence Synthetic
cuacgagaccucccgggaggucucguag

SEQ ID NO: 81 - RNA, 26 nt, Artificial Sequence
  Description of Artificial Sequence Synthetic
uacgagaccucccgggaggucucgua

SEQ ID NO: 82 - RNA, 24 nt, Artificial Sequence
  Description of Artificial Sequence Synthetic
acgagaccucccgggaggucucgu

SEQ ID NO: 83 - RNA, 19 nt, Artificial Sequence
  Description of Artificial Sequence Target Sequence
cccggggaagugguugucu

SEQ ID NO: 84 - RNA, 19 nt, Artificial Sequence
  Description of Artificial Sequence Target Sequence
gccggcggcggcgaacgag

SEQ ID NO: 85 - RNA, 19 nt, Artificial Sequence
  Description of Artificial Sequence Target Sequence
cggccgggucguuggccgg

SEQ ID NO: 86 - RNA, 19 nt, Artificial Sequence
  Description of Artificial Sequence Target Sequence
accauggucagcuacuggg

SEQ ID NO: 87 - RNA, 19 nt, Artificial Sequence
  Description of Artificial Sequence Target Sequence
gcgcgcugcucagcugucu

SEQ ID NO: 88 - RNA, 19 nt, Artificial Sequence
  Description of Artificial Sequence Target Sequence
cagcugucugcuucucaca

SEQ ID NO: 89 - RNA, 19 nt, Artificial Sequence
  Description of Artificial Sequence Target Sequence
uuuaaaaggcacccagcac

SEQ ID NO: 90 - RNA, 19 nt, Artificial Sequence
  Description of Artificial Sequence Target Sequence
cugcaguacuuuaaccuug

SEQ ID NO: 91 - RNA, 19 nt, Artificial Sequence
  Description of Artificial Sequence Target Sequence
cagcugcaaauaucuagcu

SEQ ID NO: 92 - RNA, 19 nt, Artificial Sequence
  Description of Artificial Sequence Target Sequence
uauauauuuauuagugaua

SEQ ID NO: 93 - RNA, 19 nt, Artificial Sequence
  Description of Artificial Sequence Target Sequence
uguacagugaaauccccga

SEQ ID NO: 94 - RNA, 19 nt, Artificial Sequence
  Description of Artificial Sequence Target Sequence
gagcucgucauucccugcc

SEQ ID NO: 95 - RNA, 19 nt, Artificial Sequence
  Description of Artificial Sequence Target Sequence
uuuaaaaaaguuuccacuu

SEQ ID NO: 96 - RNA, 19 nt, Artificial Sequence
  Description of Artificial Sequence Target Sequence
caauuguacugcuaccacu

SEQ ID NO: 97 - RNA, 19 nt, Artificial Sequence
  Description of Artificial Sequence Target Sequence
uguuaacaccucagugcau

SEQ ID NO: 98 - RNA, 19 nt, Artificial Sequence
  Description of Artificial Sequence Target Sequence
cauauauaugauaaagcau

SEQ ID NO: 99 - RNA, 19 nt, Artificial Sequence
  Description of Artificial Sequence Target Sequence
uaauuaucaaggacguaac

SEQ ID NO: 100 - RNA, 19 nt, Artificial Sequence
  Description of Artificial Sequence Target Sequence
uuuaaaaaccucacugcca

SEQ ID NO: 101 - RNA, 19 nt, Artificial Sequence
  Description of Artificial Sequence Target Sequence
cauaugguaucccucaacc

SEQ ID NO: 102 - RNA, 19 nt, Artificial Sequence
  Description of Artificial Sequence Target Sequence
gcuagcaccuugguugugg

SEQ ID NO: 103 - RNA, 19 nt, Artificial Sequence
  Description of Artificial Sequence Target Sequence
ucuagaauuucuggaaucu

SEQ ID NO: 104 - RNA, 19 nt, Artificial Sequence
  Description of Artificial Sequence Target Sequence
aagcuuuuauaucacagau

SEQ ID NO: 105 - RNA, 19 nt, Artificial Sequence
  Description of Artificial Sequence Target Sequence
guuaacuuggaaaaaaugc

SEQ ID NO: 106 - RNA, 19 nt, Artificial Sequence
  Description of Artificial Sequence Target Sequence
guuaacaaguucuuauaca

SEQ ID NO: 107 - RNA, 19 nt, Artificial Sequence
  Description of Artificial Sequence Target Sequence
auggccaucacuaaggagc

SEQ ID NO: 108 - RNA, 19 nt, Artificial Sequence
  Description of Artificial Sequence Target Sequence
ucaugaauguuucccugca

SEQ ID NO: 109 - RNA, 19 nt, Artificial Sequence
  Description of Artificial Sequence Target Sequence
cugcagagccaggaaugua

SEQ ID NO: 110 - RNA, 19 nt, Artificial Sequence
  Description of Artificial Sequence Target Sequence
uguauacacaggggaagaa

SEQ ID NO: 111 - RNA, 19 nt, Artificial Sequence
  Description of Artificial Sequence Target Sequence
gugaucacacaguggccau

SEQ ID NO: 112 - RNA, 19 nt, Artificial Sequence
  Description of Artificial Sequence Target Sequence
uggccaucagcaguuccac

SEQ ID NO: 113 - RNA, 19 nt, Artificial Sequence
  Description of Artificial Sequence Target Sequence
uuuaaaaacaaccacaaaa

SEQ ID NO: 114 - RNA, 19 nt, Artificial Sequence
  Description of Artificial Sequence Target Sequence
ugaucacucuaacaugcac

SEQ ID NO: 115 - RNA, 19 nt, Artificial Sequence
  Description of Artificial Sequence Target Sequence
auuauaauggacccagaug

SEQ ID NO: 116 - RNA, 19 nt, Artificial Sequence
  Description of Artificial Sequence Target Sequence
cccgggagagacuuaaacu

SEQ ID NO: 117 - RNA, 19 nt, Artificial Sequence
  Description of Artificial Sequence Target Sequence
uggccaccaucugaacgug

SEQ ID NO: 118 - RNA, 19 nt, Artificial Sequence
  Description of Artificial Sequence Target Sequence
gguuaaccugcugggagcc

SEQ ID NO: 119 - RNA, 19 nt, Artificial Sequence
  Description of Artificial Sequence Target Sequence
ccaggccuggaacaaggca

SEQ ID NO: 120 - RNA, 19 nt, Artificial Sequence
  Description of Artificial Sequence Target Sequence
aaagcuuugcgagcuccgg

SEQ ID NO: 121 - RNA, 19 nt, Artificial Sequence
  Description of Artificial Sequence Target Sequence
gagcuccggcuuucaggaa

SEQ ID NO: 122 - RNA, 19 nt, Artificial Sequence
  Description of Artificial Sequence Target Sequence
agaucugauuucuuacagu

SEQ ID NO: 123 - RNA, 19 nt, Artificial Sequence
  Description of Artificial Sequence Target Sequence
uggccagaggcauggaguu

SEQ ID NO: 124 - RNA, 19 nt, Artificial Sequence
  Description of Artificial Sequence Target Sequence
cccgggauauuuauaagaa

SEQ ID NO: 125 - RNA, 19 nt, Artificial Sequence
  Description of Artificial Sequence Target Sequence
uuauaagaaccccgauuau

SEQ ID NO: 126 - RNA, 19 nt, Artificial Sequence
  Description of Artificial Sequence Target Sequence
gagcuccugaguacucuac

SEQ ID NO: 127 - RNA, 19 nt, Artificial Sequence
  Description of Artificial Sequence Target Sequence
gaguacucuacuccugaaa

SEQ ID NO: 128 - RNA, 19 nt, Artificial Sequence
  Description of Artificial Sequence Target Sequence
uguacaacaggaugguaaa

SEQ ID NO: 129 - RNA, 19 nt, Artificial Sequence
  Description of Artificial Sequence Target Sequence
ucaugagccuggaaagaau

SEQ ID NO: 130 - RNA, 19 nt, Artificial Sequence
  Description of Artificial Sequence Target Sequence
ugaagcgcuucaccuggac

SEQ ID NO: 131 - RNA, 19 nt, Artificial Sequence
  Description of Artificial Sequence Target Sequence
aggccucgcucaagauuga

SEQ ID NO: 132 - RNA, 19 nt, Artificial Sequence
  Description of Artificial Sequence Target Sequence
cagcugugggcacgucagc

SEQ ID NO: 133 - RNA, 19 nt, Artificial Sequence
  Description of Artificial Sequence Target Sequence
ucuagaguuugacacgaag

SEQ ID NO: 134 - RNA, 19 nt, Artificial Sequence
  Description of Artificial Sequence Target Sequence
uucuagaagcacaugugua

SEQ ID NO: 135 - RNA, 19 nt, Artificial Sequence
  Description of Artificial Sequence Target Sequence
cacauguguauuuauaccc

SEQ ID NO: 136 - RNA, 19 nt, Artificial Sequence
  Description of Artificial Sequence Target Sequence
uaugcauauauaaguuuac

SEQ ID NO: 137 - RNA, 19 nt, Artificial Sequence
  Description of Artificial Sequence Target Sequence
ccaugggagccagcugcuu

SEQ ID NO: 138 - RNA, 19 nt, Artificial Sequence
  Description of Artificial Sequence Target Sequence
cagcugcuuuuugugauuu

SEQ ID NO: 139 - RNA, 19 nt, Artificial Sequence
  Description of Artificial Sequence Target Sequence
ugaucacccaaugcaucac

SEQ ID NO: 140 - RNA, 19 nt, Artificial Sequence
  Description of Artificial Sequence Target Sequence
augcaucacguaccccacu

SEQ ID NO: 141 - RNA, 19 nt, Artificial Sequence
  Description of Artificial Sequence Target Sequence
cugcagcccaaaacccagg

SEQ ID NO: 142 - RNA, 19 nt, Artificial Sequence
  Description of Artificial Sequence Target Sequence
aggccuaagacaugugagg

SEQ ID NO: 143 - RNA, 19 nt, Artificial Sequence
  Description of Artificial Sequence Target Sequence
acaugugaggaggaaaagg

SEQ ID NO: 144 - RNA, 19 nt, Artificial Sequence
  Description of Artificial Sequence Target Sequence
gggcccagccaggagcaga

SEQ ID NO: 145 - RNA, 19 nt, Artificial Sequence
  Description of Artificial Sequence Target Sequence
aaauuuuagaccuuuaccu

SEQ ID NO: 146 - RNA, 19 nt, Artificial Sequence
  Description of Artificial Sequence Target Sequence
uauuaauauauaguccaga

SEQ ID NO: 147 - RNA, 19 nt, Artificial Sequence
  Description of Artificial Sequence Target Sequence
ggcgccuacucuucagggu

SEQ ID NO: 148 - RNA, 19 nt, Artificial Sequence
  Description of Artificial Sequence Target Sequence
cugcagccagucagaagcu

SEQ ID NO: 149 - RNA, 19 nt, Artificial Sequence
  Description of Artificial Sequence Target Sequence
gagcucuaaguaaccgaag

SEQ ID NO: 150 - RNA, 19 nt, Artificial Sequence
  Description of Artificial Sequence Target Sequence
uuuaaaggcucucuguaug

SEQ ID NO: 151 - RNA, 19 nt, Artificial Sequence
  Description of Artificial Sequence Target Sequence
agaucuaaauccaaacaaa

SEQ ID NO: 152 - RNA, 19 nt, Artificial Sequence
  Description of Artificial Sequence Target Sequence
cagcuggcaauuuuauaaa

SEQ ID NO: 153 - RNA, 19 nt, Artificial Sequence
  Description of Artificial Sequence Target Sequence
uuuauaaaucagguaacug

SEQ ID NO: 154 - RNA, 19 nt, Artificial Sequence
  Description of Artificial Sequence Target Sequence
uaauuaauucuuaaucauu

SEQ ID NO: 155 - RNA, 19 nt, Artificial Sequence
  Description of Artificial Sequence Target Sequence
aauauuccaaucauuugcc

SEQ ID NO: 156 - RNA, 19 nt, Artificial Sequence
  Description of Artificial Sequence Target Sequence
gcuagccucauuuaaauug

SEQ ID NO: 157 - RNA, 19 nt, Artificial Sequence
  Description of Artificial Sequence Target Sequence
auuuaaauugauuaaagga

SEQ ID NO: 158 - RNA, 19 nt, Artificial Sequence
  Description of Artificial Sequence Target Sequence
uuuaaaguuacuuuuauac

SEQ ID NO: 159 - RNA, 19 nt, Artificial Sequence
  Description of Artificial Sequence Target Sequence
auauaugcuacagauauaa

SEQ ID NO: 160 - RNA, 19 nt, Artificial Sequence
  Description of Artificial Sequence Target Sequence
ucaugaugaauguauuuug

SEQ ID NO: 161 - RNA, 19 nt, Artificial Sequence
  Description of Artificial Sequence Target Sequence
guauaccaucuucauauaa

SEQ ID NO: 162 - RNA, 19 nt, Artificial Sequence
  Description of Artificial Sequence Target Sequence
aaauauuucuuaauuggga

SEQ ID NO: 163 - RNA, 19 nt, Artificial Sequence
  Description of Artificial Sequence Target Sequence
aaauuuuucaaaauacuaa

SEQ ID NO: 164 - RNA, 19 nt, Artificial Sequence
  Description of Artificial Sequence Target Sequence
aaauuuauccuuguuuaga

SEQ ID NO: 165 - RNA, 19 nt, Artificial Sequence
  Description of Artificial Sequence Target Sequence
aaauauuuucaauggaaaa

SEQ ID NO: 166 - RNA, 19 nt, Artificial Sequence
  Description of Artificial Sequence Target Sequence
uucgaaccuuucacuuuuu

SEQ ID NO: 167 - RNA, 19 nt, Artificial Sequence
  Description of Artificial Sequence Target Sequence
auauauuugaccaucaccc

SEQ ID NO: 168 - RNA, 19 nt, Artificial Sequence
  Description of Artificial Sequence Target Sequence
uauauauucucugcucuuu

SEQ ID NO: 169 - RNA, 6 nt, Artificial Sequence
  Description of Artificial Sequence Synthetic
cccggg

SEQ ID NO: 170 - RNA, 6 nt, Artificial Sequence
  Description of Artificial Sequence Synthetic
gccggc

SEQ ID NO: 171 - RNA, 6 nt, Artificial Sequence
  Description of Artificial Sequence Synthetic
cggccg

SEQ ID NO: 172 - RNA, 8 nt, Artificial Sequence
  Description of Artificial Sequence Synthetic
accauggu

SEQ ID NO: 173 - RNA, 6 nt, Artificial Sequence
  Description of Artificial Sequence Synthetic
gcgcgc

SEQ ID NO: 174 - RNA, 6 nt, Artificial Sequence
  Description of Artificial Sequence Synthetic
cagcug

SEQ ID NO: 175 - RNA, 6 nt, Artificial Sequence
  Description of Artificial Sequence Synthetic
uuuaaa

SEQ ID NO: 176 - RNA, 6 nt, Artificial Sequence
  Description of Artificial Sequence Synthetic
cugcag

SEQ ID NO: 177 - RNA, 6 nt, Artificial Sequence
  Description of Artificial Sequence Synthetic
cagcug

SEQ ID NO: 178 - RNA, 6 nt, Artificial Sequence
  Description of Artificial Sequence Synthetic
uauaua

SEQ ID NO: 179 - RNA, 6 nt, Artificial Sequence
  Description of Artificial Sequence Synthetic
uguaca

SEQ ID NO: 180 - RNA, 6 nt, Artificial Sequence
  Description of Artificial Sequence Synthetic
gagcuc

SEQ ID NO: 181 - RNA, 6 nt, Artificial Sequence
  Description of Artificial Sequence Synthetic
uuuaaa

SEQ ID NO: 182 - RNA, 6 nt, Artificial Sequence
  Description of Artificial Sequence Synthetic
caauug

SEQ ID NO: 183 - RNA, 8 nt, Artificial Sequence
  Description of Artificial Sequence Synthetic
uguuaaca

SEQ ID NO: 184 - RNA, 10 nt, Artificial Sequence
  Description of Artificial Sequence Synthetic
cauauauaug

SEQ ID NO: 185 - RNA, 6 nt, Artificial Sequence
  Description of Artificial Sequence Synthetic
uaauua

SEQ ID NO: 186 - RNA, 6 nt, Artificial Sequence
  Description of Artificial Sequence Synthetic
uuuaaa

SEQ ID NO: 187 - RNA, 6 nt, Artificial Sequence
  Description of Artificial Sequence Synthetic
cauaug

SEQ ID NO: 188 - RNA, 6 nt, Artificial Sequence
  Description of Artificial Sequence Synthetic
gcuagc

SEQ ID NO: 189 - RNA, 6 nt, Artificial Sequence
  Description of Artificial Sequence Synthetic
ucuaga

SEQ ID NO: 190 - RNA, 6 nt, Artificial Sequence
  Description of Artificial Sequence Synthetic
aagcuu

SEQ ID NO: 191 - RNA, 6 nt, Artificial Sequence
  Description of Artificial Sequence Synthetic
guuaac

SEQ ID NO: 192 - RNA, 6 nt, Artificial Sequence
  Description of Artificial Sequence Synthetic
guuaac

SEQ ID NO: 193 - RNA, 8 nt, Artificial Sequence
  Description of Artificial Sequence Synthetic
auggccau

SEQ ID NO: 194 - RNA, 6 nt, Artificial Sequence
  Description of Artificial Sequence Synthetic
ucauga

SEQ ID NO: 195 - RNA, 6 nt, Artificial Sequence
  Description of Artificial Sequence Synthetic
cugcag

SEQ ID NO: 196 - RNA, 8 nt, Artificial Sequence
  Description of Artificial Sequence Synthetic
uguauaca

SEQ ID NO: 197 - RNA, 8 nt, Artificial Sequence
  Description of Artificial Sequence Synthetic
gugaucac

SEQ ID NO: 198 - RNA, 6 nt, Artificial Sequence
  Description of Artificial Sequence Synthetic
uggcca

SEQ ID NO: 199 - RNA, 6 nt, Artificial Sequence
  Description of Artificial Sequence Synthetic
uuuaaa

SEQ ID NO: 200 - RNA, 6 nt, Artificial Sequence
  Description of Artificial Sequence Synthetic
ugauca

SEQ ID NO: 201 - RNA, 8 nt, Artificial Sequence
  Description of Artificial Sequence Synthetic
auuauaau

SEQ ID NO: 202 - RNA, 6 nt, Artificial Sequence
  Description of Artificial Sequence Synthetic
cccggg

SEQ ID NO: 203 - RNA, 6 nt, Artificial Sequence
  Description of Artificial Sequence Synthetic
uggcca

SEQ ID NO: 204 - RNA, 8 nt, Artificial Sequence
  Description of Artificial Sequence Synthetic
gguuaacc

SEQ ID NO: 205 - RNA, 10 nt, Artificial Sequence
  Description of Artificial Sequence Synthetic
ccaggccugg

SEQ ID NO: 206 - RNA, 8 nt, Artificial Sequence
  Description of Artificial Sequence Synthetic
aaagcuuu

SEQ ID NO: 207 - RNA, 6 nt, Artificial Sequence
  Description of Artificial Sequence Synthetic
gagcuc

SEQ ID NO: 208 - RNA, 6 nt, Artificial Sequence
  Description of Artificial Sequence Synthetic
agaucu

SEQ ID NO: 209 - RNA, 6 nt, Artificial Sequence
  Description of Artificial Sequence Synthetic
uggcca

SEQ ID NO: 210 - RNA, 6 nt, Artificial Sequence
  Description of Artificial Sequence Synthetic
cccggg

SEQ ID NO: 211 - RNA, 6 nt, Artificial Sequence
  Description of Artificial Sequence Synthetic
uuauaa

SEQ ID NO: 212 - RNA, 6 nt, Artificial Sequence
  Description of Artificial Sequence Synthetic
gagcuc

SEQ ID NO: 213 - RNA, 8 nt, Artificial Sequence
  Description of Artificial Sequence Synthetic
gaguacuc

SEQ ID NO: 214 - RNA, 6 nt, Artificial Sequence
  Description of Artificial Sequence Synthetic
uguaca

SEQ ID NO: 215 - RNA, 6 nt, Artificial Sequence
  Description of Artificial Sequence Synthetic
ucauga

SEQ ID NO: 216 - RNA, 12 nt, Artificial Sequence
  Description of Artificial Sequence Synthetic
ugaagcgcuuca

SEQ ID NO: 217 - RNA, 6 nt, Artificial Sequence
  Description of Artificial Sequence Synthetic
aggccu

SEQ ID NO: 218 - RNA, 6 nt, Artificial Sequence
  Description of Artificial Sequence Synthetic
cagcug

SEQ ID NO: 219 - RNA, 6 nt, Artificial Sequence
  Description of Artificial Sequence Synthetic
ucuaga

SEQ ID NO: 220 - RNA, 8 nt, Artificial Sequence
  Description of Artificial Sequence Synthetic
uucuagaa

SEQ ID NO: 221 - RNA, 8 nt, Artificial Sequence
  Description of Artificial Sequence Synthetic
cacaugug

SEQ ID NO: 222 - RNA, 8 nt, Artificial Sequence
  Description of Artificial Sequence Synthetic
uaugcaua

SEQ ID NO: 223 - RNA, 6 nt, Artificial Sequence
  Description of Artificial Sequence Synthetic
ccaugg

SEQ ID NO: 224 - RNA, 6 nt, Artificial Sequence
  Description of Artificial Sequence Synthetic
cagcug

SEQ ID NO: 225 - RNA, 6 nt, Artificial Sequence
  Description of Artificial Sequence Synthetic
ugauca

SEQ ID NO: 226 - RNA, 6 nt, Artificial Sequence
  Description of Artificial Sequence Synthetic
augcau

SEQ ID NO: 227 - RNA, 6 nt, Artificial Sequence
  Description of Artificial Sequence Synthetic
cugcag

SEQ ID NO: 228 - RNA, 6 nt, Artificial Sequence
  Description of Artificial Sequence Synthetic
aggccu

SEQ ID NO: 229 - RNA, 6 nt, Artificial Sequence
  Description of Artificial Sequence Synthetic
acaugu

SEQ ID NO: 230 - RNA, 6 nt, Artificial Sequence
  Description of Artificial Sequence Synthetic
gggccc

SEQ ID NO: 231 - RNA, 6 nt, Artificial Sequence
  Description of Artificial Sequence Synthetic
aaauuu

SEQ ID NO: 232 - RNA, 8 nt, Artificial Sequence
  Description of Artificial Sequence Synthetic
uauuaaua

SEQ ID NO: 233 - RNA, 6 nt, Artificial Sequence
  Description of Artificial Sequence Synthetic
ggcgcc

SEQ ID NO: 234 - RNA, 6 nt, Artificial Sequence
  Description of Artificial Sequence Synthetic
cugcag

SEQ ID NO: 235 - RNA, 6 nt, Artificial Sequence
  Description of Artificial Sequence Synthetic
gagcuc

SEQ ID NO: 236 - RNA, 6 nt, Artificial Sequence
  Description of Artificial Sequence Synthetic
uuuaaa

SEQ ID NO: 237 - RNA, 6 nt, Artificial Sequence
  Description of Artificial Sequence Synthetic
agaucu

SEQ ID NO: 238 - RNA, 6 nt, Artificial Sequence
  Description of Artificial Sequence Synthetic
cagcug

SEQ ID NO: 239 - RNA, 8 nt, Artificial Sequence
  Description of Artificial Sequence Synthetic
uuuauaaa

SEQ ID NO: 240 - RNA, 6 nt, Artificial Sequence
  Description of Artificial Sequence Synthetic
uaauua

SEQ ID NO: 241 - RNA, 6 nt, Artificial Sequence
  Description of Artificial Sequence Synthetic
aauauu

SEQ ID NO: 242 - RNA, 6 nt, Artificial Sequence
  Description of Artificial Sequence Synthetic
gcuagc

SEQ ID NO: 243 - RNA, 8 nt, Artificial Sequence
  Description of Artificial Sequence Synthetic
auuuaaau

SEQ ID NO: 244 - RNA, 6 nt, Artificial Sequence
  Description of Artificial Sequence Synthetic
uuuaaa

SEQ ID NO: 245 - RNA, 6 nt, Artificial Sequence
  Description of Artificial Sequence Synthetic
auauau

SEQ ID NO: 246 - RNA, 6 nt, Artificial Sequence
  Description of Artificial Sequence Synthetic
ucauga

SEQ ID NO: 247 - RNA, 6 nt, Artificial Sequence
  Description of Artificial Sequence Synthetic
guauac

SEQ ID NO: 248 - RNA, 8 nt, Artificial Sequence
  Description of Artificial Sequence Synthetic
aaauauuu

SEQ ID NO: 249 - RNA, 6 nt, Artificial Sequence
  Description of Artificial Sequence Synthetic
aaauuu

SEQ ID NO: 250 - RNA, 6 nt, Artificial Sequence
  Description of Artificial Sequence Synthetic
aaauuu

SEQ ID NO: 251 - RNA, 8 nt, Artificial Sequence
  Description of Artificial Sequence Synthetic
aaauauuu

SEQ ID NO: 252 - RNA, 6 nt, Artificial Sequence
  Description of Artificial Sequence Synthetic
uucgaa

SEQ ID NO: 253 - RNA, 6 nt, Artificial Sequence
  Description of Artificial Sequence Synthetic
auauau

SEQ ID NO: 254 - RNA, 6 nt, Artificial Sequence
  Description of Artificial Sequence Synthetic
uauaua

SEQ ID NO: 255 - RNA, 19 nt, Artificial Sequence
  Description of Artificial Sequence Target Sequence
gucccgggaccccgggaga

SEQ ID NO: 256 - RNA, 19 nt, Artificial Sequence
  Description of Artificial Sequence Target Sequence
cccgggagagcggucagug

SEQ ID NO: 257 - RNA, 19 nt, Artificial Sequence
  Description of Artificial Sequence Target Sequence
gcgcgccgcagaaaguccg

SEQ ID NO: 258 - RNA, 19 nt, Artificial Sequence
  Description of Artificial Sequence Target Sequence
ggauauccucuccuaccgg

SEQ ID NO: 259 - RNA, 19 nt, Artificial Sequence
  Description of Artificial Sequence Target Sequence
cugcagccgccggucggcg

SEQ ID NO: 260 - RNA, 19 nt, Artificial Sequence
  Description of Artificial Sequence Target Sequence
ggcgcccgggcucccuagc

SEQ ID NO: 261 - RNA, 19 nt, Artificial Sequence
  Description of Artificial Sequence Target Sequence
cccgggcucccuagcccug

SEQ ID NO: 262 - RNA, 19 nt, Artificial Sequence
  Description of Artificial Sequence Target Sequence
ucuagacaggcgcugggag

SEQ ID NO: 263 - RNA, 19 nt, Artificial Sequence
  Description of Artificial Sequence Target Sequence
cucgaggugcaggaugcag

SEQ ID NO: 264 - RNA, 19 nt, Artificial Sequence
  Description of Artificial Sequence Target Sequence
cccgggccgccucuguggg

SEQ ID NO: 265 - RNA, 19 nt, Artificial Sequence
  Description of Artificial Sequence Target Sequence
agaucuccauuuauugcuu

SEQ ID NO: 266 - RNA, 19 nt, Artificial Sequence
  Description of Artificial Sequence Target Sequence
uguacauuacugagaacaa

SEQ ID NO: 267 - RNA, 19 nt, Artificial Sequence
  Description of Artificial Sequence Target Sequence
ugaucagcuaugcuggcau

SEQ ID NO: 268 - RNA, 19 nt, Artificial Sequence
  Description of Artificial Sequence Target Sequence
auuaaugaugaaaguuacc

SEQ ID NO: 269 - RNA, 19 nt, Artificial Sequence
  Description of Artificial Sequence Target Sequence
uauguacauaguugucguu

SEQ ID NO: 270 - RNA, 19 nt, Artificial Sequence
  Description of Artificial Sequence Target Sequence
aagcuugucuuaaauugua

SEQ ID NO: 271 - RNA, 19 nt, Artificial Sequence
  Description of Artificial Sequence Target Sequence
uguacagcaagaacugaac

SEQ ID NO: 272 - RNA, 19 nt, Artificial Sequence
  Description of Artificial Sequence Target Sequence
cuucgaagcaucagcauaa

SEQ ID NO: 273 - RNA, 19 nt, Artificial Sequence
  Description of Artificial Sequence Target Sequence
aaauuuuugagcaccuuaa

SEQ ID NO: 274 - RNA, 19 nt, Artificial Sequence
  Description of Artificial Sequence Target Sequence
cuauagaugguguaacccg

SEQ ID NO: 275 - RNA, 19 nt, Artificial Sequence
  Description of Artificial Sequence Target Sequence
uguacaccugugcagcauc

SEQ ID NO: 276 - RNA, 19 nt, Artificial Sequence
  Description of Artificial Sequence Target Sequence
acauguacggucuaugcca

SEQ ID NO: 277 - RNA, 19 nt, Artificial Sequence
  Description of Artificial Sequence Target Sequence
uuuguacaaaugugaagcg

SEQ ID NO: 278 - RNA, 19 nt, Artificial Sequence
  Description of Artificial Sequence Target Sequence
cacgugaccagggguccug

SEQ ID NO: 279 - RNA, 19 nt, Artificial Sequence
  Description of Artificial Sequence Target Sequence
uugcaaccugacaugcagc

SEQ ID NO: 280 - RNA, 19 nt, Artificial Sequence
  Description of Artificial Sequence Target Sequence
gugcacugcagacagaucu

SEQ ID NO: 281 - RNA, 19 nt, Artificial Sequence
  Description of Artificial Sequence Target Sequence
cugcagacagaucuacguu

SEQ ID NO: 282 - RNA, 19 nt, Artificial Sequence
  Description of Artificial Sequence Target Sequence
agaucuacguuugagaacc

SEQ ID NO: 283 - RNA, 19 nt, Artificial Sequence
  Description of Artificial Sequence Target Sequence
caagcuuggcccacagccu

SEQ ID NO: 284 - RNA, 19 nt, Artificial Sequence
  Description of Artificial Sequence Target Sequence
uugcaagaacuuggauacu

SEQ ID NO: 285 - RNA, 19 nt, Artificial Sequence
  Description of Artificial Sequence Target Sequence
ugaucauggagcuuaagaa

SEQ ID NO: 286 - RNA, 19 nt, Artificial Sequence
  Description of Artificial Sequence Target Sequence
cuuaagaaugcauccuugc

SEQ ID NO: 287 - RNA, 19 nt, Artificial Sequence
  Description of Artificial Sequence Target Sequence
augcauccuugcaggacca

SEQ ID NO: 288 - RNA, 19 nt, Artificial Sequence
  Description of Artificial Sequence Target Sequence
uuuaaagauaaugagaccc

SEQ ID NO: 289 - RNA, 19 nt, Artificial Sequence
  Description of Artificial Sequence Target Sequence
aggccucuacaccugccag

SEQ ID NO: 290 - RNA, 19 nt, Artificial Sequence
  Description of Artificial Sequence Target Sequence
gcaugcaguguucuuggcu

SEQ ID NO: 291 - RNA, 19 nt, Artificial Sequence
  Description of Artificial Sequence Target Sequence
uggauccagaugaacuccc

SEQ ID NO: 292 - RNA, 19 nt, Artificial Sequence
  Description of Artificial Sequence Target Sequence
gggaauuccccagagaccg

SEQ ID NO: 293 - RNA, 19 nt, Artificial Sequence
  Description of Artificial Sequence Target Sequence
uuggccaagugauugaagc

SEQ ID NO: 294 - RNA, 19 nt, Artificial Sequence
  Description of Artificial Sequence Target Sequence
gagcucucaugucugaacu

SEQ ID NO: 295 - RNA, 19 nt, Artificial Sequence
  Description of Artificial Sequence Target Sequence
gaauucugcaaauuuggaa

SEQ ID NO: 296 - RNA, 19 nt, Artificial Sequence
  Description of Artificial Sequence Target Sequence
caaauuuggaaaccugucc

SEQ ID NO: 297 - RNA, 19 nt, Artificial Sequence
  Description of Artificial Sequence Target Sequence
gagcucagccagcucugga

SEQ ID NO: 298 - RNA, 19 nt, Artificial Sequence
  Description of Artificial Sequence Target Sequence
agaucuguauaaggacuuc

SEQ ID NO: 299 - RNA, 19 nt, Artificial Sequence
  Description of Artificial Sequence Target Sequence
ucgcgaaaguguauccaca

SEQ ID NO: 300 - RNA, 19 nt, Artificial Sequence
  Description of Artificial Sequence Target Sequence
cccgggauauuuauaaaga

SEQ ID NO: 301 - RNA, 19 nt, Artificial Sequence
  Description of Artificial Sequence Target Sequence
uuuauaaagauccagauua

SEQ ID NO: 302 - RNA, 19 nt, Artificial Sequence
  Description of Artificial Sequence Target Sequence
guguacacaauccagagug

SEQ ID NO: 303 - RNA, 19 nt, Artificial Sequence
  Description of Artificial Sequence Target Sequence
gacgucuggucuuuuggug

SEQ ID NO: 304 - RNA, 19 nt, Artificial Sequence
  Description of Artificial Sequence Target Sequence
aaauauuuuccuuaggugc

SEQ ID NO: 305 - RNA, 19 nt, Artificial Sequence
  Description of Artificial Sequence Target Sequence
gggccccugauuauacuac

SEQ ID NO: 306 - RNA, 19 nt, Artificial Sequence
  Description of Artificial Sequence Target Sequence
cuugcaagcuaaugcucag

SEQ ID NO: 307 - RNA, 19 nt, Artificial Sequence
  Description of Artificial Sequence Target Sequence
gauaucagagacuuugagc

SEQ ID NO: 308 - RNA, 19 nt, Artificial Sequence
  Description of Artificial Sequence Target Sequence
ucugcagaacaguaagcga

SEQ ID NO: 309 - RNA, 19 nt, Artificial Sequence
  Description of Artificial Sequence Target Sequence
gccggccugugaguguaaa

SEQ ID NO: 310 - RNA, 19 nt, Artificial Sequence
  Description of Artificial Sequence Target Sequence
gauaucccguuagaagaac

SEQ ID NO: 311 - RNA, 19 nt, Artificial Sequence
  Description of Artificial Sequence Target Sequence
uccggauaucacuccgaug

SEQ ID NO: 312 - RNA, 19 nt, Artificial Sequence
  Description of Artificial Sequence Target Sequence
gauaucacuccgaugacac

SEQ ID NO: 313 - RNA, 19 nt, Artificial Sequence
  Description of Artificial Sequence Target Sequence
uuuaaagcugauagagauu

SEQ ID NO: 314 - RNA, 19 nt, Artificial Sequence
  Description of Artificial Sequence Target Sequence
accgguagcacagcccaga

SEQ ID NO: 315 - RNA, 19 nt, Artificial Sequence
  Description of Artificial Sequence Target Sequence
gagcucuccuccuguuuaa

SEQ ID NO: 316 - RNA, 19 nt, Artificial Sequence
  Description of Artificial Sequence Target Sequence
uuuaaaaggaagcauccac

SEQ ID NO: 317 - RNA, 19 nt, Artificial Sequence
  Description of Artificial Sequence Target Sequence
cugcagggagccagucuuc

SEQ ID NO: 318 - RNA, 19 nt, Artificial Sequence
  Description of Artificial Sequence Target Sequence
uuuaaaaagcauuaucaug

SEQ ID NO: 319 - RNA, 19 nt, Artificial Sequence
  Description of Artificial Sequence Target Sequence
ccauggguuuagaacaaag

SEQ ID NO: 320 - RNA, 19 nt, Artificial Sequence
  Description of Artificial Sequence Target Sequence
cuuaaguguggaauucgga

SEQ ID NO: 321 - RNA, 19 nt, Artificial Sequence
  Description of Artificial Sequence Target Sequence
gaauucggauugauagaaa

SEQ ID NO: 322 - RNA, 19 nt, Artificial Sequence
  Description of Artificial Sequence Target Sequence
uaacguuaccuugcuuugg

SEQ ID NO: 323 - RNA, 19 nt, Artificial Sequence
  Description of Artificial Sequence Target Sequence
aguacuggagccugcaaau

SEQ ID NO: 324 - RNA, 19 nt, Artificial Sequence
  Description of Artificial Sequence Target Sequence
aaugcauuguguuugcucu

SEQ ID NO: 325 - RNA, 19 nt, Artificial Sequence
  Description of Artificial Sequence Target Sequence
uuauaacaucuauuguauu

SEQ ID NO: 326 - RNA, 19 nt, Artificial Sequence
  Description of Artificial Sequence Target Sequence
ugguaccauagugugaaau

SEQ ID NO: 327 - RNA, 19 nt, Artificial Sequence
  Description of Artificial Sequence Target Sequence
auauauuuauagucuguuu

SEQ ID NO: 328 - RNA, 19 nt, Artificial Sequence
  Description of Artificial Sequence Target Sequence
uaauauauuaaagccuuau

SEQ ID NO: 329 - RNA, 19 nt, Artificial Sequence
  Description of Artificial Sequence Target Sequence
uuauauauaaugaacuuug

SEQ ID NO: 330 - RNA, 19 nt, Artificial Sequence
  Description of Artificial Sequence Target Sequence
caauugaugucauuuuauu

SEQ ID NO: 331 - RNA, 10 nt, Artificial Sequence
  Description of Artificial Sequence Synthetic
gucccgggac

SEQ ID NO: 332 - RNA, 6 nt, Artificial Sequence
  Description of Artificial Sequence Synthetic
cccggg

SEQ ID NO: 333 - RNA, 6 nt, Artificial Sequence
  Description of Artificial Sequence Synthetic
gcgcgc

SEQ ID NO: 334 - RNA, 8 nt, Artificial Sequence
  Description of Artificial Sequence Synthetic
ggauaucc

SEQ ID NO: 335 - RNA, 6 nt, Artificial Sequence
  Description of Artificial Sequence Synthetic
cugcag

SEQ ID NO: 336 - RNA, 6 nt, Artificial Sequence
  Description of Artificial Sequence Synthetic
ggcgcc

SEQ ID NO: 337 - RNA, 6 nt, Artificial Sequence
  Description of Artificial Sequence Synthetic
cccggg

SEQ ID NO: 338 - RNA, 6 nt, Artificial Sequence
  Description of Artificial Sequence Synthetic
ucuaga

SEQ ID NO: 339 - RNA, 6 nt, Artificial Sequence
  Description of Artificial Sequence Synthetic
cucgag

SEQ ID NO: 340 - RNA, 6 nt, Artificial Sequence
  Description of Artificial Sequence Synthetic
cccggg

SEQ ID NO: 341 - RNA, 6 nt, Artificial Sequence
  Description of Artificial Sequence Synthetic
agaucu

SEQ ID NO: 342 - RNA, 6 nt, Artificial Sequence
  Description of Artificial Sequence Synthetic
uguaca

SEQ ID NO: 343 - RNA, 6 nt, Artificial Sequence
  Description of Artificial Sequence Synthetic
ugauca

SEQ ID NO: 344 - RNA, 6 nt, Artificial Sequence
  Description of Artificial Sequence Synthetic
auuaau

SEQ ID NO: 345 - RNA, 10 nt, Artificial Sequence
  Description of Artificial Sequence Synthetic
uauguacaua

SEQ ID NO: 346 - RNA, 6 nt, Artificial Sequence
  Description of Artificial Sequence Synthetic
aagcuu

SEQ ID NO: 347 - RNA, 6 nt, Artificial Sequence
  Description of Artificial Sequence Synthetic
uguaca

SEQ ID NO: 348 - RNA, 8 nt, Artificial Sequence
  Description of Artificial Sequence Synthetic
cuucgaag

SEQ ID NO: 349 - RNA, 6 nt, Artificial Sequence
  Description of Artificial Sequence Synthetic
aaauuu

SEQ ID NO: 350 - RNA, 6 nt, Artificial Sequence
  Description of Artificial Sequence Synthetic
cuauag

SEQ ID NO: 351 - RNA, 6 nt, Artificial Sequence
  Description of Artificial Sequence Synthetic
uguaca

SEQ ID NO: 352 - RNA, 6 nt, Artificial Sequence
  Description of Artificial Sequence Synthetic
acaugu

SEQ ID NO: 353 - RNA, 10 nt, Artificial Sequence
  Description of Artificial Sequence Synthetic
uuuguacaaa

SEQ ID NO: 354 - RNA, 6 nt, Artificial Sequence
  Description of Artificial Sequence Synthetic
cacgug

SEQ ID NO: 355 - RNA, 6 nt, Artificial Sequence
  Description of Artificial Sequence Synthetic
uugcaa

SEQ ID NO: 356 - RNA, 6 nt, Artificial Sequence
  Description of Artificial Sequence Synthetic
gugcac

SEQ ID NO: 357 - RNA, 6 nt, Artificial Sequence
  Description of Artificial Sequence Synthetic
cugcag

SEQ ID NO: 358 - RNA, 6 nt, Artificial Sequence
  Description of Artificial Sequence Synthetic
agaucu

SEQ ID NO: 359 - RNA, 8 nt, Artificial Sequence
  Description of Artificial Sequence Synthetic
caagcuug

SEQ ID NO: 360 - RNA, 6 nt, Artificial Sequence
  Description of Artificial Sequence Synthetic
uugcaa

SEQ ID NO: 361 - RNA, 6 nt, Artificial Sequence
  Description of Artificial Sequence Synthetic
ugauca

SEQ ID NO: 362 - RNA, 6 nt, Artificial Sequence
  Description of Artificial Sequence Synthetic
cuuaag

SEQ ID NO: 363 - RNA, 6 nt, Artificial Sequence
  Description of Artificial Sequence Synthetic
augcau

SEQ ID NO: 364 - RNA, 6 nt, Artificial Sequence
  Description of Artificial Sequence Synthetic
uuuaaa

SEQ ID NO: 365 - RNA, 6 nt, Artificial Sequence
  Description of Artificial Sequence Synthetic
aggccu

SEQ ID NO: 366 - RNA, 6 nt, Artificial Sequence
  Description of Artificial Sequence Synthetic
gcaugc

SEQ ID NO: 367 - RNA, 8 nt, Artificial Sequence
  Description of Artificial Sequence Synthetic
uggaucca

SEQ ID NO: 368 - RNA, 10 nt, Artificial Sequence
  Description of Artificial Sequence Synthetic
gggaauuccc

SEQ ID NO: 369 - RNA, 8 nt, Artificial Sequence
  Description of Artificial Sequence Synthetic
uuggccaa

SEQ ID NO: 370 - RNA, 6 nt, Artificial Sequence
  Description of Artificial Sequence Synthetic
gagcuc

SEQ ID NO: 371 - RNA, 6 nt, Artificial Sequence
  Description of Artificial Sequence Synthetic
gaauuc

SEQ ID NO: 372 - RNA, 8 nt, Artificial Sequence
  Description of Artificial Sequence Synthetic
caaauuug

SEQ ID NO: 373 - RNA, 6 nt, Artificial Sequence
  Description of Artificial Sequence Synthetic
gagcuc

SEQ ID NO: 374 - RNA, 6 nt, Artificial Sequence
  Description of Artificial Sequence Synthetic
agaucu

SEQ ID NO: 375 - RNA, 6 nt, Artificial Sequence
  Description of Artificial Sequence Synthetic
ucgcga

SEQ ID NO: 376 - RNA, 6 nt, Artificial Sequence
  Description of Artificial Sequence Synthetic
cccggg

SEQ ID NO: 377 - RNA, 8 nt, Artificial Sequence
  Description of Artificial Sequence Synthetic
uuuauaaa

SEQ ID NO: 378 - RNA, 8 nt, Artificial Sequence
  Description of Artificial Sequence Synthetic
guguacac

SEQ ID NO: 379 - RNA, 6 nt, Artificial Sequence
  Description of Artificial Sequence Synthetic
gacguc

SEQ ID NO: 380 - RNA, 8 nt, Artificial Sequence
  Description of Artificial Sequence Synthetic
aaauauuu

SEQ ID NO: 381 - RNA, 6 nt, Artificial Sequence
  Description of Artificial Sequence Synthetic
gggccc

SEQ ID NO: 382 - RNA, 8 nt, Artificial Sequence
  Description of Artificial Sequence Synthetic
cuugcaag

SEQ ID NO: 383 - RNA, 6 nt, Artificial Sequence
  Description of Artificial Sequence Synthetic
gauauc

SEQ ID NO: 384 - RNA, 8 nt, Artificial Sequence
  Description of Artificial Sequence Synthetic
ucugcaga

SEQ ID NO: 385 - RNA, 6 nt, Artificial Sequence
  Description of Artificial Sequence Synthetic
gccggc

SEQ ID NO: 386 - RNA, 6 nt, Artificial Sequence
  Description of Artificial Sequence Synthetic
gauauc

SEQ ID NO: 387 - RNA, 6 nt, Artificial Sequence
  Description of Artificial Sequence Synthetic
uccgga

SEQ ID NO: 388 - RNA, 6 nt, Artificial Sequence
  Description of Artificial Sequence Synthetic
gauauc

SEQ ID NO: 389 - RNA, 6 nt, Artificial Sequence
  Description of Artificial Sequence Synthetic
uuuaaa

SEQ ID NO: 390 - RNA, 6 nt, Artificial Sequence
  Description of Artificial Sequence Synthetic
accggu

SEQ ID NO: 391 - RNA, 6 nt, Artificial Sequence
  Description of Artificial Sequence Synthetic
gagcuc

SEQ ID NO: 392 - RNA, 6 nt, Artificial Sequence
  Description of Artificial Sequence Synthetic
uuuaaa

SEQ ID NO: 393 - RNA, 6 nt, Artificial Sequence
  Description of Artificial Sequence Synthetic
cugcag

SEQ ID NO: 394 - RNA, 6 nt, Artificial Sequence
  Description of Artificial Sequence Synthetic
uuuaaa

SEQ ID NO: 395 - RNA, 6 nt, Artificial Sequence
  Description of Artificial Sequence Synthetic
ccaugg

SEQ ID NO: 396 - RNA, 6 nt, Artificial Sequence
  Description of Artificial Sequence Synthetic
cuuaag

SEQ ID NO: 397 - RNA, 6 nt, Artificial Sequence
  Description of Artificial Sequence Synthetic
gaauuc

SEQ ID NO: 398 - RNA, 8 nt, Artificial Sequence
  Description of Artificial Sequence Synthetic
uaacguua

SEQ ID NO: 399 - RNA, 6 nt, Artificial Sequence
  Description of Artificial Sequence Synthetic
aguacu

SEQ ID NO: 400 - RNA, 8 nt, Artificial Sequence
  Description of Artificial Sequence Synthetic
aaugcauu

SEQ ID NO: 401 - RNA, 6 nt, Artificial Sequence
  Description of Artificial Sequence Synthetic
uuauaa

SEQ ID NO: 402 - RNA, 8 nt, Artificial Sequence
  Description of Artificial Sequence Synthetic
ugguacca

SEQ ID NO: 403 - RNA, 6 nt, Artificial Sequence
  Description of Artificial Sequence Synthetic
auauau

SEQ ID NO: 404 - RNA, 10 nt, Artificial Sequence
  Description of Artificial Sequence Synthetic
uaauauauua

SEQ ID NO: 405 - RNA, 10 nt, Artificial Sequence
  Description of Artificial Sequence Synthetic
uuauauauaa

SEQ ID NO: 406 - RNA, 6 nt, Artificial Sequence
  Description of Artificial Sequence Synthetic
caauug

SEQ ID NO: 407 - RNA, 19 nt, Artificial Sequence
  Description of Artificial Sequence Target Sequence
gcuagcaccagcgcucugu

SEQ ID NO: 408 - RNA, 19 nt, Artificial Sequence
  Description of Artificial Sequence Target Sequence
agcgcucugucgggaggcg

SEQ ID NO: 409 - RNA, 19 nt, Artificial Sequence
  Description of Artificial Sequence Target Sequence
gaccggucagcggacucac

SEQ ID NO: 410 - RNA, 19 nt, Artificial Sequence
  Description of Artificial Sequence Target Sequence
uuuuaaaacuguauuguuu

SEQ ID NO: 411 - RNA, 19 nt, Artificial Sequence
  Description of Artificial Sequence Target Sequence
gagcuccagagagaagucg

SEQ ID NO: 412 - RNA, 19 nt, Artificial Sequence
  Description of Artificial Sequence Target Sequence
gcgcgcgggcgugcgagca

SEQ ID NO: 413 - RNA, 19 nt, Artificial Sequence
  Description of Artificial Sequence Target Sequence
gggaucccgcagcugacca

SEQ ID NO: 414 - RNA, 19 nt, Artificial Sequence
  Description of Artificial Sequence Target Sequence
cagcugaccagucgcgcug

SEQ ID NO: 415 - RNA, 19 nt, Artificial Sequence
  Description of Artificial Sequence Target Sequence
ccggccggcggcggacagu

SEQ ID NO: 416 - RNA, 19 nt, Artificial Sequence
  Description of Artificial Sequence Target Sequence
gccggcggcggacagugga

SEQ ID NO: 417 - RNA, 19 nt, Artificial Sequence
  Description of Artificial Sequence Target Sequence
ccgcgggcaggggccggag

SEQ ID NO: 418 - RNA, 19 nt, Artificial Sequence
  Description of Artificial Sequence Target Sequence
ccgcgcgggggaagccgag

SEQ ID NO: 419 - RNA, 19 nt, Artificial Sequence
  Description of Artificial Sequence Target Sequence
gcuagcucgggccgggagg

SEQ ID NO: 420 - RNA, 19 nt, Artificial Sequence
  Description of Artificial Sequence Target Sequence
ugcgcagacagugcuccag

SEQ ID NO: 421 - RNA, 19 nt, Artificial Sequence
  Description of Artificial Sequence Target Sequence
cgcgcgcgcuccccaggcc

SEQ ID NO: 422 - RNA, 19 nt, Artificial Sequence
  Description of Artificial Sequence Target Sequence
ggcccgggccucgggccgg

SEQ ID NO: 423 - RNA, 19 nt, Artificial Sequence
  Description of Artificial Sequence Target Sequence
ggcgccgaggagagcgggc

SEQ ID NO: 424 - RNA, 19 nt, Artificial Sequence
  Description of Artificial Sequence Target Sequence
gccggccccggucgggccu

SEQ ID NO: 425 - RNA, 19 nt, Artificial Sequence
  Description of Artificial Sequence Target Sequence
ccauggcagaaggaggagg

SEQ ID NO: 426 - RNA, 19 nt, Artificial Sequence
  Description of Artificial Sequence Target Sequence
uuguacaagauccgcagac

SEQ ID NO: 427 - RNA, 19 nt, Artificial Sequence
  Description of Artificial Sequence Target Sequence
uugcaaggcgaggcagcuu

SEQ ID NO: 428 - RNA, 6 nt, Artificial Sequence
  Description of Artificial Sequence Synthetic
gcuagc

SEQ ID NO: 429 - RNA, 6 nt, Artificial Sequence
  Description of Artificial Sequence Synthetic
agcgcu

SEQ ID NO: 430 - RNA, 8 nt, Artificial Sequence
  Description of Artificial Sequence Synthetic
gaccgguc

SEQ ID NO: 431 - RNA, 8 nt, Artificial Sequence
  Description of Artificial Sequence Synthetic
uuuuaaaa

SEQ ID NO: 432 - RNA, 6 nt, Artificial Sequence
  Description of Artificial Sequence Synthetic
gagcuc

SEQ ID NO: 433 - RNA, 6 nt, Artificial Sequence
  Description of Artificial Sequence Synthetic
gcgcgc

SEQ ID NO: 434 - RNA, 8 nt, Artificial Sequence
  Description of Artificial Sequence Synthetic
gggauccc

SEQ ID NO: 435 - RNA, 6 nt, Artificial Sequence
  Description of Artificial Sequence Synthetic
cagcug

SEQ ID NO: 436 - RNA, 8 nt, Artificial Sequence
  Description of Artificial Sequence Synthetic
ccggccgg

SEQ ID NO: 437 - RNA, 6 nt, Artificial Sequence
  Description of Artificial Sequence Synthetic
gccggc

SEQ ID NO: 438 - RNA, 6 nt, Artificial Sequence
  Description of Artificial Sequence Synthetic
ccgcgg

SEQ ID NO: 439 - RNA, 8 nt, Artificial Sequence
  Description of Artificial Sequence Synthetic
ccgcgcgg

SEQ ID NO: 440 - RNA, 6 nt, Artificial Sequence
  Description of Artificial Sequence Synthetic
gcuagc

SEQ ID NO: 441 - RNA, 6 nt, Artificial Sequence
  Description of Artificial Sequence Synthetic
ugcgca

SEQ ID NO: 442 - RNA, 8 nt, Artificial Sequence
  Description of Artificial Sequence Synthetic
cgcgcgcg

SEQ ID NO: 443 - RNA, 10 nt, Artificial Sequence
  Description of Artificial Sequence Synthetic
ggcccgggcc

SEQ ID NO: 444 - RNA, 6 nt, Artificial Sequence
  Description of Artificial Sequence Synthetic
ggcgcc

SEQ ID NO: 445 - RNA, 6 nt, Artificial Sequence
  Description of Artificial Sequence Synthetic
gccggc

SEQ ID NO: 446 - RNA, 6 nt, Artificial Sequence
  Description of Artificial Sequence Synthetic
ccaugg

SEQ ID NO: 447 - RNA, 8 nt, Artificial Sequence
  Description of Artificial Sequence Synthetic
uuguacaa

SEQ ID NO: 448 - RNA, 6 nt, Artificial Sequence
  Description of Artificial Sequence Synthetic
uugcaa

SEQ ID NO: 449 - RNA, 19 nt, Artificial Sequence
  Description of Artificial Sequence Target Sequence
cggccgggccgggccgggc

SEQ ID NO: 450 - RNA, 19 nt, Artificial Sequence
  Description of Artificial Sequence Target Sequence
ccauggaggcggcggucgc

SEQ ID NO: 451 - RNA, 19 nt, Artificial Sequence
  Description of Artificial Sequence Target Sequence
cccgggggcgacggcguua

SEQ ID NO: 452 - RNA, 19 nt, Artificial Sequence
  Description of Artificial Sequence Target Sequence
uguacaaaagacaauuuua

SEQ ID NO: 453 - RNA, 19 nt, Artificial Sequence
  Description of Artificial Sequence Target Sequence
cucgagauaggccguuugu

SEQ ID NO: 454 - RNA, 19 nt, Artificial Sequence
  Description of Artificial Sequence Target Sequence
gugcacccucuucaaaaac

SEQ ID NO: 455 - RNA, 19 nt, Artificial Sequence
  Description of Artificial Sequence Target Sequence
auugcaauaaaauagaacu

SEQ ID NO: 456 - RNA, 19 nt, Artificial Sequence
  Description of Artificial Sequence Target Sequence
cagcugucauugcuggacc

SEQ ID NO: 457 - RNA, 19 nt, Artificial Sequence
  Description of Artificial Sequence Target Sequence
caauugcgagaacuauugu

SEQ ID NO: 458 - RNA, 19 nt, Artificial Sequence
  Description of Artificial Sequence Target Sequence
cucuagagaagaacguucg

SEQ ID NO: 459 - RNA, 19 nt, Artificial Sequence
  Description of Artificial Sequence Target Sequence
gaacguucgugguuccgug

SEQ ID NO: 460 - RNA, 19 nt, Artificial Sequence
  Description of Artificial Sequence Target Sequence
ucaugaaaacauccuggga

SEQ ID NO: 461 - RNA, 19 nt, Artificial Sequence
  Description of Artificial Sequence Target Sequence
ucaugagcauggaucccuu

SEQ ID NO: 462 - RNA, 19 nt, Artificial Sequence
  Description of Artificial Sequence Target Sequence
ggaucccuuuuugauuacu

SEQ ID NO: 463 - RNA, 19 nt, Artificial Sequence
  Description of Artificial Sequence Target Sequence
gguacccaaggaaagccag

SEQ ID NO: 464 - RNA, 19 nt, Artificial Sequence
  Description of Artificial Sequence Target Sequence
gaauucaugaagauuacca

SEQ ID NO: 465 - RNA, 19 nt, Artificial Sequence
  Description of Artificial Sequence Target Sequence
uucaugaagauuaccaacu

SEQ ID NO: 466 - RNA, 19 nt, Artificial Sequence
  Description of Artificial Sequence Target Sequence
cagaucugcuccuggguuu

SEQ ID NO: 467 - RNA, 19 nt, Artificial Sequence
  Description of Artificial Sequence Target Sequence
gugcacuaugaacgcuucu

SEQ ID NO: 468 - RNA, 19 nt, Artificial Sequence
  Description of Artificial Sequence Target Sequence
uuuuuaaaaagaugauugc

SEQ ID NO: 469 - RNA, 19 nt, Artificial Sequence
  Description of Artificial Sequence Target Sequence
acaugucuuauuacuaaag

SEQ ID NO: 470 - RNA, 19 nt, Artificial Sequence
  Description of Artificial Sequence Target Sequence
cuauaguuuuucaggaucu

SEQ ID NO: 471 - RNA, 19 nt, Artificial Sequence
  Description of Artificial Sequence Target Sequence
uuauaaaacucuuaucuug

SEQ ID NO: 472 - RNA, 19 nt, Artificial Sequence
  Description of Artificial Sequence Target Sequence
caauuguauuuuguauacu

SEQ ID NO: 473 - RNA, 19 nt, Artificial Sequence
  Description of Artificial Sequence Target Sequence
guauacuauuauuguucuu

SEQ ID NO: 474 - RNA, 6 nt, Artificial Sequence
  Description of Artificial Sequence Synthetic
cggccg

SEQ ID NO: 475 - RNA, 6 nt, Artificial Sequence
  Description of Artificial Sequence Synthetic
ccaugg

SEQ ID NO: 476 - RNA, 6 nt, Artificial Sequence
  Description of Artificial Sequence Synthetic
cccggg

SEQ ID NO: 477 - RNA, 6 nt, Artificial Sequence
  Description of Artificial Sequence Synthetic
uguaca

SEQ ID NO: 478 - RNA, 6 nt, Artificial Sequence
  Description of Artificial Sequence Synthetic
cucgag

SEQ ID NO: 479 - RNA, 6 nt, Artificial Sequence
  Description of Artificial Sequence Synthetic
gugcac

SEQ ID NO: 480 - RNA, 8 nt, Artificial Sequence
  Description of Artificial Sequence Synthetic
auugcaau

SEQ ID NO: 481 - RNA, 6 nt, Artificial Sequence
  Description of Artificial Sequence Synthetic
cagcug

SEQ ID NO: 482 - RNA, 6 nt, Artificial Sequence
  Description of Artificial Sequence Synthetic
caauug

SEQ ID NO: 483 - RNA, 8 nt, Artificial Sequence
  Description of Artificial Sequence Synthetic
cucuagag

SEQ ID NO: 484 - RNA, 8 nt, Artificial Sequence
  Description of Artificial Sequence Synthetic
gaacguuc

SEQ ID NO: 485 - RNA, 6 nt, Artificial Sequence
  Description of Artificial Sequence Synthetic
ucauga

SEQ ID NO: 486 - RNA, 6 nt, Artificial Sequence
  Description of Artificial Sequence Synthetic
ucauga

SEQ ID NO: 487 - RNA, 6 nt, Artificial Sequence
  Description of Artificial Sequence Synthetic
ggaucc

SEQ ID NO: 488 - RNA, 6 nt, Artificial Sequence
  Description of Artificial Sequence Synthetic
gguacc

SEQ ID NO: 489 - RNA, 6 nt, Artificial Sequence
  Description of Artificial Sequence Synthetic
gaauuc

SEQ ID NO: 490 - RNA, 8 nt, Artificial Sequence
  Description of Artificial Sequence Synthetic
uucaugaa

SEQ ID NO: 491 - RNA, 8 nt, Artificial Sequence
  Description of Artificial Sequence Synthetic
cagaucug

SEQ ID NO: 492 - RNA, 6 nt, Artificial Sequence
  Description of Artificial Sequence Synthetic
gugcac

SEQ ID NO: 493 - RNA, 10 nt, Artificial Sequence
  Description of Artificial Sequence Synthetic
uuuuuaaaaa

SEQ ID NO: 494 - RNA, 6 nt, Artificial Sequence
  Description of Artificial Sequence Synthetic
acaugu

SEQ ID NO: 495 - RNA, 6 nt, Artificial Sequence
  Description of Artificial Sequence Synthetic
cuauag

SEQ ID NO: 496 - RNA, 6 nt, Artificial Sequence
  Description of Artificial Sequence Synthetic
uuauaa

SEQ ID NO: 497 - RNA, 6 nt, Artificial Sequence
  Description of Artificial Sequence Synthetic
caauug

SEQ ID NO: 498 - RNA, 6 nt, Artificial Sequence
  Description of Artificial Sequence Synthetic
guauac

SEQ ID NO: 499 - RNA, 19 nt, Artificial Sequence
  Description of Artificial Sequence Target Sequence
caauggccauugacagaag

SEQ ID NO: 500 - RNA, 19 nt, Artificial Sequence
  Description of Artificial Sequence Target Sequence
uuuuaaaagaaaagggggg

SEQ ID NO: 501 - RNA, 19 nt, Artificial Sequence
  Description of Artificial Sequence Target Sequence
uuuuaaaagaaaagggggg

SEQ ID NO: 502 - RNA, 19 nt, Artificial Sequence
  Description of Artificial Sequence Target Sequence
ccaugcauggacaaguaga

SEQ ID NO: 503 - RNA, 19 nt, Artificial Sequence
  Description of Artificial Sequence Target Sequence
augcauuaggaaucauuca

SEQ ID NO: 504 - RNA, 19 nt, Artificial Sequence
  Description of Artificial Sequence Target Sequence
aaaauuuucggguuuauua

SEQ ID NO: 505 - RNA, 19 nt, Artificial Sequence
  Description of Artificial Sequence Target Sequence
caauuggagaagugaauua

SEQ ID NO: 506 - RNA, 19 nt, Artificial Sequence
  Description of Artificial Sequence Target Sequence
cuauagguacaguauuagu

SEQ ID NO: 507 - RNA, 19 nt, Artificial Sequence
  Description of Artificial Sequence Target Sequence
aaauuucaaaaauugggcc

SEQ ID NO: 508 - RNA, 19 nt, Artificial Sequence
  Description of Artificial Sequence Target Sequence
gcuuaagccucaauaaagc

SEQ ID NO: 509 - RNA, 19 nt, Artificial Sequence
  Description of Artificial Sequence Target Sequence
gcuuaagccucaauaaagc

SEQ ID NO: 510 - RNA, 19 nt, Artificial Sequence
  Description of Artificial Sequence Target Sequence
uuuaaaugcauggguaaaa

SEQ ID NO: 511 - RNA, 19 nt, Artificial Sequence
  Description of Artificial Sequence Target Sequence
gauaucaguacaaugugcu

SEQ ID NO: 512 - RNA, 19 nt, Artificial Sequence
  Description of Artificial Sequence Target Sequence
aaauuuaucaagagccauu

SEQ ID NO: 513 - RNA, 19 nt, Artificial Sequence
  Description of Artificial Sequence Target Sequence
uguacagaaauggaaaagg

SEQ ID NO: 514 - RNA, 19 nt, Artificial Sequence
  Description of Artificial Sequence Target Sequence
acauguggaaaaauaacau

SEQ ID NO: 515 - RNA, 19 nt, Artificial Sequence
  Description of Artificial Sequence Target Sequence
guugcaacucacagucugg

SEQ ID NO: 516 - RNA, 19 nt, Artificial Sequence
  Description of Artificial Sequence Target Sequence
gguaccagcacacaaagga

SEQ ID NO: 517 - RNA, 19 nt, Artificial Sequence
  Description of Artificial Sequence Target Sequence
cagcugugauaaaugucag

SEQ ID NO: 518 - RNA, 19 nt, Artificial Sequence
  Description of Artificial Sequence Target Sequence
augcauauuuuucaguucc

SEQ ID NO: 519 - RNA, 19 nt, Artificial Sequence
  Description of Artificial Sequence Target Sequence
aagcuugccuugagugcuu

SEQ ID NO: 520 - RNA, 19 nt, Artificial Sequence
  Description of Artificial Sequence Target Sequence
aguacuaaauggagaaaau

SEQ ID NO: 521 - RNA, 19 nt, Artificial Sequence
  Description of Artificial Sequence Target Sequence
uuuaaacuacccauacaaa

SEQ ID NO: 522 - RNA, 19 nt, Artificial Sequence
  Description of Artificial Sequence Target Sequence
aagcuugccuugagugcuu

SEQ ID NO: 523 - RNA, 19 nt, Artificial Sequence
  Description of Artificial Sequence Target Sequence
uacuaguacccuucaggaa

SEQ ID NO: 524 - RNA, 19 nt, Artificial Sequence
  Description of Artificial Sequence Target Sequence
ccuaggaaaaagggcuguu

SEQ ID NO: 525 - RNA, 19 nt, Artificial Sequence
  Description of Artificial Sequence Target Sequence
cagcuggacugucaaugac

SEQ ID NO: 526 - RNA, 19 nt, Artificial Sequence
  Description of Artificial Sequence Target Sequence
augcauggguaaaaguagu

SEQ ID NO: 527 - RNA, 19 nt, Artificial Sequence
  Description of Artificial Sequence Target Sequence
uaaauuuaaacuacccaua

SEQ ID NO: 528 - RNA, 19 nt, Artificial Sequence
  Description of Artificial Sequence Target Sequence
cagaucugagccugggagc

SEQ ID NO: 529 - RNA, 19 nt, Artificial Sequence
  Description of Artificial Sequence Target Sequence
aaauuuguacagaaaugga

SEQ ID NO: 530 - RNA, 19 nt, Artificial Sequence
  Description of Artificial Sequence Target Sequence
uguacacauuuagaaggaa

SEQ ID NO: 531 - RNA, 19 nt, Artificial Sequence
  Description of Artificial Sequence Target Sequence
uuauauaaauauaaaguag

SEQ ID NO: 532 - RNA, 19 nt, Artificial Sequence
  Description of Artificial Sequence Target Sequence
cagaucugagccugggagc

SEQ ID NO: 533 - RNA, 19 nt, Artificial Sequence
  Description of Artificial Sequence Target Sequence
ugguaccaguuagagaaag

SEQ ID NO: 534 - RNA, 19 nt, Artificial Sequence
  Description of Artificial Sequence Target Sequence
aaauuuucccauuaguccu

SEQ ID NO: 535 - RNA, 19 nt, Artificial Sequence
  Description of Artificial Sequence Target Sequence
ucuuaagacagcaguacaa

SEQ ID NO: 536 - RNA, 19 nt, Artificial Sequence
  Description of Artificial Sequence Target Sequence
uguacacauggaauuaggc

SEQ ID NO: 537 - RNA, 19 nt, Artificial Sequence
  Description of Artificial Sequence Target Sequence
gggccccuaggaaaaaggg

SEQ ID NO: 538 - RNA, 19 nt, Artificial Sequence
  Description of Artificial Sequence Target Sequence
aguauacugcauuuaccau

SEQ ID NO: 539 - RNA, 19 nt, Artificial Sequence
  Description of Artificial Sequence Target Sequence
uuuuaaaagcauugggacc

SEQ ID NO: 540 - RNA, 19 nt, Artificial Sequence
  Description of Artificial Sequence Target Sequence
cuauagugcugccagaaaa

SEQ ID NO: 541 - RNA, 19 nt, Artificial Sequence
  Description of Artificial Sequence Target Sequence
uuuaaauuuucccauuagu

SEQ ID NO: 542 - RNA, 19 nt, Artificial Sequence
  Description of Artificial Sequence Target Sequence
auauauagaagcagaaguu

SEQ ID NO: 543 - RNA, 19 nt, Artificial Sequence
  Description of Artificial Sequence Target Sequence
uauauaaaaauauucauaa

SEQ ID NO: 544 - RNA, 19 nt, Artificial Sequence
  Description of Artificial Sequence Target Sequence
uuuaaacaccaugcuaaac

SEQ ID NO: 545 - RNA, 19 nt, Artificial Sequence
  Description of Artificial Sequence Target Sequence
uggccaguaaaaacaauac

SEQ ID NO: 546 - RNA, 19 nt, Artificial Sequence
  Description of Artificial Sequence Target Sequence
caauuguggcaaagaaggg

SEQ ID NO: 547 - RNA, 19 nt, Artificial Sequence
  Description of Artificial Sequence Target Sequence
caguacuggauguggguga

SEQ ID NO: 548 - RNA, 19 nt, Artificial Sequence
  Description of Artificial Sequence Target Sequence
aaaauuuuaacauguggaa

SEQ ID NO: 549 - RNA, 19 nt, Artificial Sequence
  Description of Artificial Sequence Target Sequence
aauauucauaaugauagua

SEQ ID NO: 550 - RNA, 19 nt, Artificial Sequence
  Description of Artificial Sequence Target Sequence
aagcagcugcuuuuugccu

SEQ ID NO: 551 - RNA, 19 nt, Artificial Sequence
  Description of Artificial Sequence Target Sequence
agguaccuuuaagaccaau

SEQ ID NO: 552 - RNA, 19 nt, Artificial Sequence
  Description of Artificial Sequence Target Sequence
aagcagcugcuuuuugccu

SEQ ID NO: 553 - RNA, 19 nt, Artificial Sequence
  Description of Artificial Sequence Target Sequence
gcgcgcacggcaagaggcg

SEQ ID NO: 554 - RNA, 19 nt, Artificial Sequence
  Description of Artificial Sequence Target Sequence
accgguucuauaaaacucu

SEQ ID NO: 555 - RNA, 19 nt, Artificial Sequence
  Description of Artificial Sequence Target Sequence
uuuaaaaaucugaaaacag

SEQ ID NO: 556 - RNA, 19 nt, Artificial Sequence
  Description of Artificial Sequence Target Sequence
aagcuucacaggagguaaa

SEQ ID NO: 557 - RNA, 19 nt, Artificial Sequence
  Description of Artificial Sequence Target Sequence
agaauucugcaacaacugc

SEQ ID NO: 558 - RNA, 19 nt, Artificial Sequence
  Description of Artificial Sequence Target Sequence
aauauuccaaaguagcaug

SEQ ID NO: 559 - RNA, 19 nt, Artificial Sequence
  Description of Artificial Sequence Target Sequence
augcaugaggauauaauca

SEQ ID NO: 560 - RNA, 19 nt, Artificial Sequence
  Description of Artificial Sequence Target Sequence
aaaauuuugacuagcggag

SEQ ID NO: 561 - RNA, 19 nt, Artificial Sequence
  Description of Artificial Sequence Target Sequence
auuauauaauacaguagca

SEQ ID NO: 562 - RNA, 19 nt, Artificial Sequence
  Description of Artificial Sequence Target Sequence
aauuaauuguacaagaccc

SEQ ID NO: 563 - RNA, 19 nt, Artificial Sequence
  Description of Artificial Sequence Target Sequence
agaucuuagccacuuuuua

SEQ ID NO: 564 - RNA, 19 nt, Artificial Sequence
  Description of Artificial Sequence Target Sequence
acaggccuguccaaaggua

SEQ ID NO: 565 - RNA, 19 nt, Artificial Sequence
  Description of Artificial Sequence Target Sequence
aauauugguggaaucuccu

SEQ ID NO: 566 - RNA, 19 nt, Artificial Sequence
  Description of Artificial Sequence Target Sequence
gagcucucuggcuaacuag

SEQ ID NO: 567 - RNA, 19 nt, Artificial Sequence
  Description of Artificial Sequence Target Sequence
gagcucucuggcuaacuag

SEQ ID NO: 568 - RNA, 19 nt, Artificial Sequence
  Description of Artificial Sequence Target Sequence
aguacuauuuuuagaugga

SEQ ID NO: 569 - RNA, 19 nt, Artificial Sequence
  Description of Artificial Sequence Target Sequence
gauauccacugaccuuugg

SEQ ID NO: 570 - RNA, 19 nt, Artificial Sequence
  Description of Artificial Sequence Target Sequence
acauguggcaggaaguagg

SEQ ID NO: 571 - RNA, 19 nt, Artificial Sequence
  Description of Artificial Sequence Target Sequence
gauauccacugaccuuugg

SEQ ID NO: 572 - RNA, 19 nt, Artificial Sequence
  Description of Artificial Sequence Target Sequence
gagcucucucgacgcagga

SEQ ID NO: 573 - RNA, 19 nt, Artificial Sequence
  Description of Artificial Sequence Target Sequence
uuguacaagacccaacaac

SEQ ID NO: 574 - RNA, 19 nt, Artificial Sequence
  Description of Artificial Sequence Target Sequence
ggcgcccgaacagggacuu

SEQ ID NO: 575 - RNA, 19 nt, Artificial Sequence
  Description of Artificial Sequence Target Sequence
aauauuacagggcugcuau

SEQ ID NO: 576 - RNA, 19 nt, Artificial Sequence
  Description of Artificial Sequence Target Sequence
ugucgacauagcagaauag

SEQ ID NO: 577 - RNA, 19 nt, Artificial Sequence
  Description of Artificial Sequence Target Sequence
aauauuaagacaaagaaaa

SEQ ID NO: 578 - RNA, 19 nt, Artificial Sequence
  Description of Artificial Sequence Target Sequence
ccuaggugugaauaucaag

SEQ ID NO: 579 - RNA, 19 nt, Artificial Sequence
  Description of Artificial Sequence Target Sequence
uguacaaaugucagcacag

SEQ ID NO: 580 - RNA, 19 nt, Artificial Sequence
  Description of Artificial Sequence Target Sequence
uuauaaacauguggcagga

SEQ ID NO: 581 - RNA, 19 nt, Artificial Sequence
  Description of Artificial Sequence Target Sequence
cauaugauacagagguaca

SEQ ID NO: 582 - RNA, 19 nt, Artificial Sequence
  Description of Artificial Sequence Target Sequence
gcuauagcaguagcugagg

SEQ ID NO: 583 - RNA, 19 nt, Artificial Sequence
  Description of Artificial Sequence Target Sequence
cuuaagaaugaagcuguua

SEQ ID NO: 584 - RNA, 19 nt, Artificial Sequence
  Description of Artificial Sequence Target Sequence
agaucuucagaccuggagg

SEQ ID NO: 585 - RNA, 19 nt, Artificial Sequence
  Description of Artificial Sequence Target Sequence
cuauagugcagaacaucca

SEQ ID NO: 586 - RNA, 19 nt, Artificial Sequence
  Description of Artificial Sequence Target Sequence
gauauccuugaucugugga

SEQ ID NO: 587 - RNA, 19 nt, Artificial Sequence
  Description of Artificial Sequence Target Sequence
auuaauaacaccaaaaaag

SEQ ID NO: 588 - RNA, 19 nt, Artificial Sequence
  Description of Artificial Sequence Target Sequence
gauauccuugaucugugga

SEQ ID NO: 589 - RNA, 19 nt, Artificial Sequence
  Description of Artificial Sequence Target Sequence
uuuauaaaagauggauaau

SEQ ID NO: 590 - RNA, 19 nt, Artificial Sequence
  Description of Artificial Sequence Target Sequence
agaucuggccuuccuacaa

SEQ ID NO: 591 - RNA, 19 nt, Artificial Sequence
  Description of Artificial Sequence Target Sequence
cagcuguagaucuuagcca

SEQ ID NO: 592 - RNA, 19 nt, Artificial Sequence
  Description of Artificial Sequence Target Sequence
ugaucagauacucauagaa

SEQ ID NO: 593 - RNA, 19 nt, Artificial Sequence
  Description of Artificial Sequence Target Sequence
ggaauucccuacaaucccc

SEQ ID NO: 594 - RNA, 19 nt, Artificial Sequence
  Description of Artificial Sequence Target Sequence
aaauuuauaaaagauggau

SEQ ID NO: 595 - RNA, 19 nt, Artificial Sequence
  Description of Artificial Sequence Target Sequence
aagcuucucuaucaaagca

SEQ ID NO: 596 - RNA, 19 nt, Artificial Sequence
  Description of Artificial Sequence Target Sequence
uccggaguacuucaagaac

SEQ ID NO: 597 - RNA, 19 nt, Artificial Sequence
  Description of Artificial Sequence Target Sequence
cauauguauguuucaggga

SEQ ID NO: 598 - RNA, 19 nt, Artificial Sequence
  Description of Artificial Sequence Target Sequence
ccauggcuuagggcaacau

SEQ ID NO: 599 - RNA, 19 nt, Artificial Sequence
  Description of Artificial Sequence Target Sequence
uccggaguacuucaagaac

SEQ ID NO: 600 - RNA, 19 nt, Artificial Sequence
  Description of Artificial Sequence Target Sequence
aguacuucaagaacugcug

SEQ ID NO: 601 - RNA, 19 nt, Artificial Sequence
  Description of Artificial Sequence Target Sequence
gguaccuguguggaaggaa

SEQ ID NO: 602 - RNA, 19 nt, Artificial Sequence
  Description of Artificial Sequence Target Sequence
aguacuucaagaacugcug

SEQ ID NO: 603 - RNA, 19 nt, Artificial Sequence
  Description of Artificial Sequence Target Sequence
cugcagaaugggauagagu

SEQ ID NO: 604 - RNA, 19 nt, Artificial Sequence
  Description of Artificial Sequence Target Sequence
aucgaugggaaaaaauucg

SEQ ID NO: 605 - RNA, 19 nt, Artificial Sequence
  Description of Artificial Sequence Target Sequence
aagcuuuagacaagauaga

SEQ ID NO: 606 - RNA, 19 nt, Artificial Sequence
  Description of Artificial Sequence Target Sequence
uuuaaagugcacugauuug

SEQ ID NO: 607 - RNA, 19 nt, Artificial Sequence
  Description of Artificial Sequence Target Sequence
cagcugacacaggacacag

SEQ ID NO: 608 - RNA, 19 nt, Artificial Sequence
  Description of Artificial Sequence Target Sequence
uacauguaaugcaaccuau

SEQ ID NO: 609 - RNA, 19 nt, Artificial Sequence
  Description of Artificial Sequence Target Sequence
aagcuuaauacacuccuua

SEQ ID NO: 610 - RNA, 19 nt, Artificial Sequence
  Description of Artificial Sequence Target Sequence
agugcacugauuugaagaa

SEQ ID NO: 611 - RNA, 19 nt, Artificial Sequence
  Description of Artificial Sequence Target Sequence
augcauuuuuuuauaaacu

SEQ ID NO: 612 - RNA, 19 nt, Artificial Sequence
  Description of Artificial Sequence Target Sequence
uuuaaaacagauagcuagc

SEQ ID NO: 613 - RNA, 19 nt, Artificial Sequence
  Description of Artificial Sequence Target Sequence
uagcuagcaaauuaagaga

SEQ ID NO: 614 - RNA, 19 nt, Artificial Sequence
  Description of Artificial Sequence Target Sequence
ucucgagaccuagaaaaac

SEQ ID NO: 615 - RNA, 19 nt, Artificial Sequence
  Description of Artificial Sequence Target Sequence
uuuuaaaauuagcaggaag

SEQ ID NO: 616 - RNA, 19 nt, Artificial Sequence
  Description of Artificial Sequence Target Sequence
uuuauaaacuugauauaau

SEQ ID NO: 617 - RNA, 19 nt, Artificial Sequence
  Description of Artificial Sequence Target Sequence
aguacugaagggucaaaua

SEQ ID NO: 618 - RNA, 19 nt, Artificial Sequence
  Description of Artificial Sequence Target Sequence
ggauccuuagcacuuaucu

SEQ ID NO: 619 - RNA, 19 nt, Artificial Sequence
  Description of Artificial Sequence Target Sequence
aaggccuuauuaggacaca

SEQ ID NO: 620 - RNA, 19 nt, Artificial Sequence
  Description of Artificial Sequence Target Sequence
cagcugaacacaucuguag

SEQ ID NO: 621 - RNA, 19 nt, Artificial Sequence
  Description of Artificial Sequence Target Sequence
gcaugccagggaguaggag

SEQ ID NO: 622 - RNA, 19 nt, Artificial Sequence
  Description of Artificial Sequence Target Sequence
gagcucaucagaacaguca

SEQ ID NO: 623 - RNA, 19 nt, Artificial Sequence
  Description of Artificial Sequence Target Sequence
uaauuagaucugucaauuu

SEQ ID NO: 624 - RNA, 19 nt, Artificial Sequence
  Description of Artificial Sequence Target Sequence
agaucugucaauuucacgg

SEQ ID NO: 625 - RNA, 19 nt, Artificial Sequence
  Description of Artificial Sequence Target Sequence
ucaugacaaaagccuuagg

SEQ ID NO: 626 - RNA, 19 nt, Artificial Sequence
  Description of Artificial Sequence Target Sequence
ugcaugcagggccuaucgc

SEQ ID NO: 627 - RNA, 19 nt, Artificial Sequence
  Description of Artificial Sequence Target Sequence
caauauuggagucaggagc

SEQ ID NO: 628 - RNA, 19 nt, Artificial Sequence
  Description of Artificial Sequence Target Sequence
cucgagagcugcauccgga

SEQ ID NO: 629 - RNA, 19 nt, Artificial Sequence
  Description of Artificial Sequence Target Sequence
uccuaggauuuagcuccau

SEQ ID NO: 630 - RNA, 19 nt, Artificial Sequence
  Description of Artificial Sequence Target Sequence
cucgagagcugcauccgga

SEQ ID NO: 631 - RNA, 19 nt, Artificial Sequence
  Description of Artificial Sequence Target Sequence
agaucuaucuggcaugggu

SEQ ID NO: 632 - RNA, 19 nt, Artificial Sequence
  Description of Artificial Sequence Target Sequence
aguacuuggaguaauagua

SEQ ID NO: 633 - RNA, 19 nt, Artificial Sequence
  Description of Artificial Sequence Target Sequence
aguacuuugaguacugaag

SEQ ID NO: 634 - RNA, 12 nt, Artificial Sequence
  Description of Artificial Sequence Synthetic
caauggccauug

SEQ ID NO: 635 - RNA, 8 nt, Artificial Sequence
  Description of Artificial Sequence Synthetic
uuuuaaaa

SEQ ID NO: 636 - RNA, 8 nt, Artificial Sequence
  Description of Artificial Sequence Synthetic
uuuuaaaa

SEQ ID NO: 637 - RNA, 10 nt, Artificial Sequence
  Description of Artificial Sequence Synthetic
ccaugcaugg

SEQ ID NO: 638 - RNA, 6 nt, Artificial Sequence
  Description of Artificial Sequence Synthetic
augcau

SEQ ID NO: 639 - RNA, 8 nt, Artificial Sequence
  Description of Artificial Sequence Synthetic
aaaauuuu

SEQ ID NO: 640 - RNA, 6 nt, Artificial Sequence
  Description of Artificial Sequence Synthetic
caauug

SEQ ID NO: 641 - RNA, 6 nt, Artificial Sequence
  Description of Artificial Sequence Synthetic
cuauag

SEQ ID NO: 642 - RNA, 6 nt, Artificial Sequence
  Description of Artificial Sequence Synthetic
aaauuu

SEQ ID NO: 643 - RNA, 8 nt, Artificial Sequence
  Description of Artificial Sequence Synthetic
gcuuaagc

SEQ ID NO: 644 - RNA, 8 nt, Artificial Sequence
  Description of Artificial Sequence Synthetic
gcuuaagc

SEQ ID NO: 645 - RNA, 6 nt, Artificial Sequence
  Description of Artificial Sequence Synthetic
uuuaaa

SEQ ID NO: 646 - RNA, 6 nt, Artificial Sequence
  Description of Artificial Sequence Synthetic
gauauc

SEQ ID NO: 647 - RNA, 6 nt, Artificial Sequence
  Description of Artificial Sequence Synthetic
aaauuu

SEQ ID NO: 648 - RNA, 6 nt, Artificial Sequence
  Description of Artificial Sequence Synthetic
uguaca

SEQ ID NO: 649 - RNA, 6 nt, Artificial Sequence
  Description of Artificial Sequence Synthetic
acaugu

SEQ ID NO: 650 - RNA, 8 nt, Artificial Sequence
  Description of Artificial Sequence Synthetic
guugcaac

SEQ ID NO: 651 - RNA, 6 nt, Artificial Sequence
  Description of Artificial Sequence Synthetic
gguacc

SEQ ID NO: 652 - RNA, 6 nt, Artificial Sequence
  Description of Artificial Sequence Synthetic
cagcug

SEQ ID NO: 653 - RNA, 6 nt, Artificial Sequence
  Description of Artificial Sequence Synthetic
augcau

SEQ ID NO: 654 - RNA, 6 nt, Artificial Sequence
  Description of Artificial Sequence Synthetic
aagcuu

SEQ ID NO: 655 - RNA, 6 nt, Artificial Sequence
  Description of Artificial Sequence Synthetic
aguacu

SEQ ID NO: 656 - RNA, 6 nt, Artificial Sequence
  Description of Artificial Sequence Synthetic
uuuaaa

SEQ ID NO: 657 - RNA, 6 nt, Artificial Sequence
  Description of Artificial Sequence Synthetic
aagcuu

SEQ ID NO: 658 - RNA, 8 nt, Artificial Sequence
  Description of Artificial Sequence Synthetic
uacuagua

SEQ ID NO: 659 - RNA, 6 nt, Artificial Sequence
  Description of Artificial Sequence Synthetic
ccuagg

SEQ ID NO: 660 - RNA, 6 nt, Artificial Sequence
  Description of Artificial Sequence Synthetic
cagcug

SEQ ID NO: 661 - RNA, 6 nt, Artificial Sequence
  Description of Artificial Sequence Synthetic
augcau

SEQ ID NO: 662 - RNA, 8 nt, Artificial Sequence
  Description of Artificial Sequence Synthetic
uaaauuua

SEQ ID NO: 663 - RNA, 8 nt, Artificial Sequence
  Description of Artificial Sequence Synthetic
cagaucug

SEQ ID NO: 664 - RNA, 6 nt, Artificial Sequence
  Description of Artificial Sequence Synthetic
aaauuu

SEQ ID NO: 665 - RNA, 6 nt, Artificial Sequence
  Description of Artificial Sequence Synthetic
uguaca

SEQ ID NO: 666 - RNA, 8 nt, Artificial Sequence
  Description of Artificial Sequence Synthetic
uuauauaa

SEQ ID NO: 667 - RNA, 8 nt, Artificial Sequence
  Description of Artificial Sequence Synthetic
cagaucug

SEQ ID NO: 668 - RNA, 8 nt, Artificial Sequence
  Description of Artificial Sequence Synthetic
ugguacca

SEQ ID NO: 669 - RNA, 6 nt, Artificial Sequence
  Description of Artificial Sequence Synthetic
aaauuu

SEQ ID NO: 670 - RNA, 8 nt, Artificial Sequence
  Description of Artificial Sequence Synthetic
ucuuaaga

SEQ ID NO: 671 - RNA, 6 nt, Artificial Sequence
  Description of Artificial Sequence Synthetic
uguaca

SEQ ID NO: 672 - RNA, 6 nt, Artificial Sequence
  Description of Artificial Sequence Synthetic
gggccc

SEQ ID NO: 673 - RNA, 8 nt, Artificial Sequence
  Description of Artificial Sequence Synthetic
aguauacu

SEQ ID NO: 674 - RNA, 8 nt, Artificial Sequence
  Description of Artificial Sequence Synthetic
uuuuaaaa

SEQ ID NO: 675 - RNA, 6 nt, Artificial Sequence
  Description of Artificial Sequence Synthetic
cuauag

SEQ ID NO: 676 - RNA, 6 nt, Artificial Sequence
  Description of Artificial Sequence Synthetic
uuuaaa

SEQ ID NO: 677 - RNA, 6 nt, Artificial Sequence
  Description of Artificial Sequence Synthetic
auauau

SEQ ID NO: 678 - RNA, 6 nt, Artificial Sequence
  Description of Artificial Sequence Synthetic
uauaua

SEQ ID NO: 679 - RNA, 6 nt, Artificial Sequence
  Description of Artificial Sequence Synthetic
uuuaaa

SEQ ID NO: 680 - RNA, 6 nt, Artificial Sequence
  Description of Artificial Sequence Synthetic
uggcca

SEQ ID NO: 681 - RNA, 6 nt, Artificial Sequence
  Description of Artificial Sequence Synthetic
caauug

SEQ ID NO: 682 - RNA, 8 nt, Artificial Sequence
  Description of Artificial Sequence Synthetic
caguacug

SEQ ID NO: 683 - RNA, 8 nt, Artificial Sequence
  Description of Artificial Sequence Synthetic
aaaauuuu

SEQ ID NO: 684 - RNA, 6 nt, Artificial Sequence
  Description of Artificial Sequence Synthetic
aauauu

SEQ ID NO: 685 - RNA, 12 nt, Artificial Sequence
  Description of Artificial Sequence Synthetic
aagcagcugcuu

SEQ ID NO: 686 - RNA, 8 nt, Artificial Sequence
  Description of Artificial Sequence Synthetic
agguaccu

SEQ ID NO: 687 - RNA, 12 nt, Artificial Sequence
  Description of Artificial Sequence Synthetic
aagcagcugcuu

SEQ ID NO: 688 - RNA, 6 nt, Artificial Sequence
  Description of Artificial Sequence Synthetic
gcgcgc

SEQ ID NO: 689 - RNA, 6 nt, Artificial Sequence
  Description of Artificial Sequence Synthetic
accggu

SEQ ID NO: 690 - RNA, 6 nt, Artificial Sequence
  Description of Artificial Sequence Synthetic
uuuaaa

SEQ ID NO: 691 - RNA, 6 nt, Artificial Sequence
  Description of Artificial Sequence Synthetic
aagcuu

SEQ ID NO: 692 - RNA, 8 nt, Artificial Sequence
  Description of Artificial Sequence Synthetic
agaauucu

SEQ ID NO: 693 - RNA, 6 nt, Artificial Sequence
  Description of Artificial Sequence Synthetic
aauauu

SEQ ID NO: 694 - RNA, 6 nt, Artificial Sequence
  Description of Artificial Sequence Synthetic
augcau

SEQ ID NO: 695 - RNA, 8 nt, Artificial Sequence
  Description of Artificial Sequence Synthetic
aaaauuuu

SEQ ID NO: 696 - RNA, 10 nt, Artificial Sequence
  Description of Artificial Sequence Synthetic
auuauauaau

SEQ ID NO: 697 - RNA, 8 nt, Artificial Sequence
  Description of Artificial Sequence Synthetic
aauuaauu

SEQ ID NO: 698 - RNA, 6 nt, Artificial Sequence
  Description of Artificial Sequence Synthetic
agaucu

SEQ ID NO: 699 - RNA, 10 nt, Artificial Sequence
  Description of Artificial Sequence Synthetic
acaggccugu

SEQ ID NO: 700 - RNA, 6 nt, Artificial Sequence
  Description of Artificial Sequence Synthetic
aauauu

SEQ ID NO: 701 - RNA, 6 nt, Artificial Sequence
  Description of Artificial Sequence Synthetic
gagcuc

SEQ ID NO: 702 - RNA, 6 nt, Artificial Sequence
  Description of Artificial Sequence Synthetic
gagcuc

SEQ ID NO: 703 - RNA, 6 nt, Artificial Sequence
  Description of Artificial Sequence Synthetic
aguacu

SEQ ID NO: 704 - RNA, 6 nt, Artificial Sequence
  Description of Artificial Sequence Synthetic
gauauc

SEQ ID NO: 705 - RNA, 6 nt, Artificial Sequence
  Description of Artificial Sequence Synthetic
acaugu

SEQ ID NO: 706 - RNA, 6 nt, Artificial Sequence
  Description of Artificial Sequence Synthetic
gauauc

SEQ ID NO: 707 - RNA, 6 nt, Artificial Sequence
  Description of Artificial Sequence Synthetic
gagcuc

SEQ ID NO: 708 - RNA, 8 nt, Artificial Sequence
  Description of Artificial Sequence Synthetic
uuguacaa

SEQ ID NO: 709 - RNA, 6 nt, Artificial Sequence
  Description of Artificial Sequence Synthetic
ggcgcc

SEQ ID NO: 710 - RNA, 6 nt, Artificial Sequence
  Description of Artificial Sequence Synthetic
aauauu

SEQ ID NO: 711 - RNA, 8 nt, Artificial Sequence
  Description of Artificial Sequence Synthetic
ugucgaca

SEQ ID NO: 712 - RNA, 6 nt, Artificial Sequence
  Description of Artificial Sequence Synthetic
aauauu

SEQ ID NO: 713 - RNA, 6 nt, Artificial Sequence
  Description of Artificial Sequence Synthetic
ccuagg

SEQ ID NO: 714 - RNA, 6 nt, Artificial Sequence
  Description of Artificial Sequence Synthetic
uguaca

SEQ ID NO: 715 - RNA, 6 nt, Artificial Sequence
  Description of Artificial Sequence Synthetic
uuauaa

SEQ ID NO: 716 - RNA, 6 nt, Artificial Sequence
  Description of Artificial Sequence Synthetic
cauaug

SEQ ID NO: 717 - RNA, 8 nt, Artificial Sequence
  Description of Artificial Sequence Synthetic
gcuauagc

SEQ ID NO: 718 - RNA, 6 nt, Artificial Sequence
  Description of Artificial Sequence Synthetic
cuuaag

SEQ ID NO: 719 - RNA, 6 nt, Artificial Sequence
  Description of Artificial Sequence Synthetic
agaucu

SEQ ID NO: 720 - RNA, 6 nt, Artificial Sequence
  Description of Artificial Sequence Synthetic
cuauag

SEQ ID NO: 721 - RNA, 6 nt, Artificial Sequence
  Description of Artificial Sequence Synthetic
gauauc

SEQ ID NO: 722 - RNA, 6 nt, Artificial Sequence
  Description of Artificial Sequence Synthetic
auuaau

SEQ ID NO: 723 - RNA, 6 nt, Artificial Sequence
  Description of Artificial Sequence Synthetic
gauauc

SEQ ID NO: 724 - RNA, 8 nt, Artificial Sequence
  Description of Artificial Sequence Synthetic
uuuauaaa

SEQ ID NO: 725 - RNA, 6 nt, Artificial Sequence
  Description of Artificial Sequence Synthetic
agaucu

SEQ ID NO: 726 - RNA, 6 nt, Artificial Sequence
  Description of Artificial Sequence Synthetic
cagcug

SEQ ID NO: 727 - RNA, 6 nt, Artificial Sequence
  Description of Artificial Sequence Synthetic
ugauca

SEQ ID NO: 728 - RNA, 8 nt, Artificial Sequence
  Description of Artificial Sequence Synthetic
ggaauucc

SEQ ID NO: 729 - RNA, 6 nt, Artificial Sequence
  Description of Artificial Sequence Synthetic
aaauuu

SEQ ID NO: 730 - RNA, 6 nt, Homo Sapiens
aagcuu

SEQ ID NO: 731 - RNA, 6 nt, Artificial Sequence
  Description of Artificial Sequence Synthetic
uccgga

SEQ ID NO: 732 - RNA, 6 nt, Artificial Sequence
  Description of Artificial Sequence Synthetic
cauaug

SEQ ID NO: 733 - RNA, 6 nt, Artificial Sequence
  Description of Artificial Sequence Synthetic
ccaugg

SEQ ID NO: 734 - RNA, 6 nt, Artificial Sequence
  Description of Artificial Sequence Synthetic
uccgga

SEQ ID NO: 735 - RNA, 6 nt, Artificial Sequence
  Description of Artificial Sequence Synthetic
aguacu

SEQ ID NO: 736 - RNA, 6 nt, Artificial Sequence
  Description of Artificial Sequence Synthetic
gguacc

SEQ ID NO: 737 - RNA, 6 nt, Artificial Sequence
  Description of Artificial Sequence Synthetic
aguacu

SEQ ID NO: 738 - RNA, 6 nt, Artificial Sequence
  Description of Artificial Sequence Synthetic
cugcag

SEQ ID NO: 739 - RNA, 6 nt, Artificial Sequence
  Description of Artificial Sequence Synthetic
aucgau

SEQ ID NO: 740 - RNA, 6 nt, Artificial Sequence
  Description of Artificial Sequence Synthetic
aagcuu

SEQ ID NO: 741 - RNA, 6 nt, Artificial Sequence
  Description of Artificial Sequence Synthetic
uuuaaa

SEQ ID NO: 742 - RNA, 6 nt, Artificial Sequence
  Description of Artificial Sequence Synthetic
cagcug

SEQ ID NO: 743 - RNA, 8 nt, Artificial Sequence
  Description of Artificial Sequence Synthetic
uacaugua

SEQ ID NO: 744 - RNA, 6 nt, Artificial Sequence
  Description of Artificial Sequence Synthetic
aagcuu

SEQ ID NO: 745 - RNA, 8 nt, Artificial Sequence
  Description of Artificial Sequence Synthetic
agugcacu

SEQ ID NO: 746 - RNA, 6 nt, Artificial Sequence
  Description of Artificial Sequence Synthetic
augcau

SEQ ID NO: 747 - RNA, 6 nt, Artificial Sequence
  Description of Artificial Sequence Synthetic
uuuaaa

SEQ ID NO: 748 - RNA, 6 nt, Artificial Sequence
  Description of Artificial Sequence Synthetic
uagcua

SEQ ID NO: 749 - RNA, 8 nt, Artificial Sequence
  Description of Artificial Sequence Synthetic
ucucgaga

SEQ ID NO: 750 - RNA, 8 nt, Artificial Sequence
  Description of Artificial Sequence Synthetic
uuuuaaaa

SEQ ID NO: 751 - RNA, 8 nt, Artificial Sequence
  Description of Artificial Sequence Synthetic
uuuauaaa

SEQ ID NO: 752 - RNA, 6 nt, Artificial Sequence
  Description of Artificial Sequence Synthetic
aguacu

SEQ ID NO: 753 - RNA, 6 nt, Artificial Sequence
  Description of Artificial Sequence Synthetic
ggaucc

SEQ ID NO: 754 - RNA, 8 nt, Artificial Sequence
  Description of Artificial Sequence Synthetic
aaggccuu

SEQ ID NO: 755 - RNA, 6 nt, Artificial Sequence
  Description of Artificial Sequence Synthetic
cagcug

SEQ ID NO: 756 - RNA, 6 nt, Artificial Sequence
  Description of Artificial Sequence Synthetic
gcaugc

SEQ ID NO: 757 - RNA, 6 nt, Artificial Sequence
  Description of Artificial Sequence Synthetic
gagcuc

SEQ ID NO: 758 - RNA, 6 nt, Artificial Sequence
  Description of Artificial Sequence Synthetic
uaauua

SEQ ID NO: 759 - RNA, 6 nt, Artificial Sequence
  Description of Artificial Sequence Synthetic
agaucu

SEQ ID NO: 760 - RNA, 6 nt, Artificial Sequence
  Description of Artificial Sequence Synthetic
ucauga

SEQ ID NO: 761 - RNA, 8 nt, Artificial Sequence
  Description of Artificial Sequence Synthetic
ugcaugca

SEQ ID NO: 762 - RNA, 8 nt, Artificial Sequence
  Description of Artificial Sequence Synthetic
caauauug

SEQ ID NO: 763 - RNA, 6 nt, Artificial Sequence
  Description of Artificial Sequence Synthetic
cucgag

SEQ ID NO: 764 - RNA, 8 nt, Artificial Sequence
  Description of Artificial Sequence Synthetic
uccuagga

SEQ ID NO: 765 - RNA, 6 nt, Artificial Sequence
  Description of Artificial Sequence Synthetic
cucgag

SEQ ID NO: 766 - RNA, 6 nt, Artificial Sequence
  Description of Artificial Sequence Synthetic
agaucu

SEQ ID NO: 767 - RNA, 6 nt, Artificial Sequence
  Description of Artificial Sequence Synthetic
aguacu

SEQ ID NO: 768 - RNA, 6 nt, Artificial Sequence
  Description of Artificial Sequence Synthetic
aguacu

SEQ ID NO: 769 - RNA, 14 nt, Artificial Sequence
  Description of Artificial Sequence  Target Sequence
auauaucuauuucg

SEQ ID NO: 770 - RNA, 14 nt, Artificial Sequence
  Description of Artificial Sequence  Complement to Target Sequence
cgaaauaguauaua

SEQ ID NO: 771 - RNA, 22 nt, Artificial Sequence
  Description of Artificial Sequence  appended target/complement
cgaaauaguauauacuauuucg

SEQ ID NO: 772 - DNA, 24 bp, Artificial Sequence
  Description of Artificial Sequence  Synthetic
cgaaauaguauauacuauuucgtt

(19) United States
(12) Patent Application Publication
(10) Pub. No.: US 2005/0233329 A1
(43) Pub. Date: Oct. 20, 2005

(54) Inhibition of gene expression using duplex forming oligonucleotides

(75) Inventors: James McSwiggen, Boulder, CO (US); Narendra K. Vaish, Denver, CO (US); Shawn Zinnen, Denver, CO (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Sirna Therapeutics, Inc., Boulder, CO (US)

(21) Appl. No.: 10/727,780

(22) Filed: Dec. 3, 2003

Related U.S. Application Data
(63) Continuation-in-part of application No. 10/720,448, filed on Nov. 24, 2003.
Application No. 10/720,448 is a continuation-in-part of application No. 10/693,059, filed on Oct. 23, 2003.
Application No. 10/693,059 is a continuation-in-part of application No. 10/444,853, filed on May 23, 2003.
Application No. 10/444,853 is a continuation-in-part of application No. PCT/US03/05346, filed on Feb. 20, 2003.
Application No. 10/444,853 is a continuation-in-part of application No. PCT/US03/05028, filed on Feb. 20, 2003.
(60) Provisional application No. 60/358,580, filed on Feb. 20, 2002.
(60) Provisional application No. 60/358,580, filed on Feb. 20, 2002.
(60) Provisional application No. 60/363,124, filed on Mar. 11, 2002.
(60) Provisional application No. 60/363,124, filed on Mar. 11, 2002.
(60) Provisional application No. 60/386,782, filed on Jun. 6, 2002.
(60) Provisional application No. 60/386,782, filed on Jun. 6, 2002.
(60) Provisional application No. 60/406,784, filed on Aug. 29, 2002.
(60) Provisional application No. 60/406,784, filed on Aug. 29, 2002.
(60) Provisional application No. 60/408,378, filed on Sep. 5, 2002.
(60) Provisional application No. 60/408,378, filed on Sep. 5, 2002.
(60) Provisional application No. 60/409,293, filed on Sep. 9, 2002.
(60) Provisional application No. 60/409,293, filed on Sep. 9, 2002.
(60) Provisional application No. 60/440,129, filed on Jan. 15, 2003.
(60) Provisional application No. 60/440,129, filed on Jan. 15, 2003.

(51) Int. Cl. C12Q 1/68; C07H 21/04

(52) U.S. Cl. 435/6; 536/23.1

(57) ABSTRACT

The present invention concerns methods and nucleic acid based reagents useful in modulating gene expression in a variety of applications, including use in therapeutic, veterinary, agricultural, diagnostic, target validation, and genomic discovery applications. Specifically, the invention relates to double strand forming oligonucleotides (DFO) that can self assemble to form double stranded oligonucleotides, such as short interfering nucleic acid (siNA), short interfering RNA (siRNA) molecules, and modulate gene expression, for example by RNA interference (RNAi). The self complementary DFO nucleic acid molecules are useful in the treatment of any disease or condition that responds to modulation of gene expression or activity in a cell, tissue, or organism.

### FIELD OF THE INVENTION

The present invention concerns methods and reagents useful in modulating gene expression in a variety of applications, including use in therapeutic, veterinary, agricultural, diagnostic, target validation, and genomic discovery applications. Specifically, the invention relates to self complementary duplex forming oligonucleotides (DFO) that modulate gene expression and methods of generating such self complementary duplex forming oligonucleotides.

### BACKGROUND OF THE INVENTION

The following is a discussion of relevant art pertaining to nucleic acid molecules that moduate gene expression. The discussion is provided only for understanding of the invention that follows. The summary is not an admission that any of the work described below is prior art to the claimed invention.

Various single strand, double strand, and triple strand nucleic acid molecules are presently known that possess biological activity. Examples of single strand nucleic acid molecules that have biologic activity to mediate alteration of gene expression include antisense nucleic acid molecules, enzymatic nucleic acid molecules or ribozymes, and 2′-5′-oligoadenylate nucleic acid molecules. Examples of triple strand nucleic acid molecules that have biologic activity to mediate alteration of gene expression include triplex forming oligonucleotides. Examples of double strand nucleic acid molecules that have biologic activity to mediate alteration of gene expression include dsRNA and siRNA. For example, interferon mediated induction of double stranded protein kinase PKR is known to be activated in a non-sequence specific manner by long double stranded RNA (see for example Wu and Kaufman, 1997, J. Biol. Chem., 272, 1921-6). This pathway shares a common feature with the 2′,5′-linked oligoadenylate (2-5A) system in mediating RNA cleavage via RNaseL (see for example Cole et al., 1997, J. Biol. Chem., 272, 19187-92). Whereas these responses are intrinsically sequence-non-specific, inhibition of gene expression via short interfering RNA mediated RNA interference (RNAi) is known to be highly sequence specific (see for example Elbashir et al., 2001, Nature, 411, 494-498).

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Hamilton et al., supra; Zamore et al., 2000, Cell, 101, 25-33; Berstein et al., 2001, Nature, 409, 363). Short interfering RNAs derived from dicer activity are typically about 21 to about 23 nucleotides in length and comprise about 19 base pair duplexes (Hamilton et al., supra; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

RNAi has been studied in a variety of systems. Fire et al., 1998, Nature, 391, 806, were the first to observe RNAi in C. elegatis. Bahramian and Zarbl, 1999, Molecular and Cellular Biology, 19, 274-283 and Wianny and Goetz, 1999, Nature Cell Biol., 2, 70, describe RNAi mediated by dsRNA in mammalian systems. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J., 20, 6877) has revealed certain requirements for siRNA length, structure, chemical composition, and sequence that are essential to mediate efficient RNAi activity. These studies have shown that 21-nucleotide siRNA duplexes are most active when containing 3′-terminal dinucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the guide sequence (Elbashir et al., 2001, EMBO J, 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have shown that replacing the 3′-terminal nucleotide overhanging segments of a 21-mer siRNA duplex having two-nucleotide 3′-overhangs with deoxyribonucleotides does not have an adverse effect on RNAi activity. Replacing up to four nucleotides on each end of the siRNA with deoxyribonucleotides has been reported to be well tolerated, whereas complete substitution with deoxyribonucleotides results in no RNAi activity (Elbashir et al., 2001, EMBO J., 20, 6877). In addition, Elbashir et al., supra, also report that substitution of siRNA with 2′-O-methyl nucleotides completely abolishes RNAi activity. Li et al., International PCT Publication No. WO 00/44914, and Beach et al., International PCT Publication No. WO 01/68836 preliminarily suggest that siRNA may include modifications to either the phosphate-sugar backbone or the nucleoside to include at least one of a nitrogen or sulfur heteroatom, however, neither application postulates to what extent such modifications would be tolerated in siRNA molecules, nor provides any further guidance or examples of such modified siRNA. Kreutzer et al., Canadian Patent Application No. 2,359,180, also describe certain chemical modifications for use in dsRNA constructs in order to counteract activation of double-stranded RNA-dependent protein kinase PKR, specifically 2′-amino or 2′-O-methyl nucleotides, and nucleotides containing a 2′-O or 4′-C methylene bridge. However, Kreutzer et al. similarly fails to provide examples or guidance as to what extent these modifications would be tolerated in siRNA molecules.

Parrish et al., 2000, Molecular Cell, 6, 1077-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that RNAs with two phosphorothioate modified bases also had substantial decreases in effectiveness as RNAi. Further, Parrish et al. reported that phosphorothioate modification of more than two residues greatly destabilized the RNAs in vitro such that interference activities could not be assayed. Id. at 1081. The authors also tested certain modifications at the 2′-position of the nucleotide sugar in the long siRNA transcripts and found that substituting deoxynucleotides for ribonucleotides produced a substantial decrease in interference activity, especially in the case of Uridine to Thymidine and/or Cytidine to deoxy-Cytidine substitutions. Id. In addition, the authors tested certain base modifications, including substituting, in sense and antisense strands of the siRNA, 4-thiouracil, 5-bromouracil, 5-iodouracil, and 3-(aminoallyl)uracil for uracil, and inosine for guanosine. Whereas 4-thiouracil and 5-bromouracil substitution appeared to be tolerated, Parrish reported that inosine produced a substantial decrease in interference activity when incorporated in either strand. Parrish also reported that incorporation of 5-iodouracil and 3-(aminoallyl)uracil in the antisense strand resulted in a substantial decrease in RNAi activity as well.

The use of longer dsRNA has been described. For example, Beach et al., International PCT Publication No. WO 01/68836, describes specific methods for attenuating gene expression using endogenously-derived dsRNA. Tuschl et al., International PCT Publication No. WO 01/75164, describe a Drosophila in vitro RNAi system and the use of specific siRNA molecules for certain functional genomic and certain therapeutic applications; although Tuschl, 2001, Chem. Biochem., 2, 239-245, doubts that RNAi can be used to cure genetic diseases or viral infection due to the danger of activating interferon response. Li et al., International PCT Publication No. WO 00/44914, describe the use of specific dsRNAs for attenuating the expression of certain target genes. Zernicka-Goetz et al., International PCT Publication No. WO 01/36646, describe certain methods for inhibiting the expression of particular genes in mammalian cells using certain dsRNA molecules. Fire et al., International PCT Publication No. WO 99/32619, describe particular methods for introducing certain dsRNA molecules into cells for use in inhibiting gene expression. Plaetinck et al., International PCT Publication No. WO 00/01846, describe certain methods for identifying specific genes responsible for conferring a particular phenotype in a cell using specific dsRNA molecules. Mello et al., International PCT Publication No. WO 01/29058, describe the identification of specific genes involved in dsRNA-mediated RNAi. Deschamps Depaillette et al., International PCT Publication No. WO 99/07409, describe specific compositions consisting of particular dsRNA molecules combined with certain anti-viral agents. Waterhouse et al., International PCT Publication No. 99/53050, describe certain methods for decreasing the phenotypic expression of a nucleic acid in plant cells using certain dsRNAs. Driscoll et al., International PCT Publication No. WO 01/49844, describe specific DNA constructs for use in facilitating gene silencing in targeted organisms.

Others have reported on various RNAi and gene-silencing systems. For example, Parrish et al., 2000, Molecular Cell, 6, 1077-1087, describe specific chemically-modified siRNA constructs targeting the unc-22 gene of C. elegans. Grossniklaus, International PCT Publication No. WO 01/38551, describes certain methods for regulating polycomb gene expression in plants using certain dsRNAs. Churikov et al., International PCT Publication No. WO 01/42443, describe certain methods for modifying genetic characteristics of an organism using certain dsRNAs. Cogoni et al., International PCT Publication No. WO 01/53475, describe certain methods for isolating a Neurospora silencing gene and uses thereof. Reed et al., International PCT Publication No. WO 01/68836, describe certain methods for gene silencing in plants. Honer et al., International PCT Publication No. WO 01/70944, describe certain methods of drug screening using transgenic nematodes as Parkinson's Disease models using certain dsRNAs. Deak et al., International PCT Publication No. WO 01/72774, describe certain Drosophila-derived gene products that may be related to RNAi in Drosophila. Arndt et al., International PCT Publication No. WO 01/92513 describe certain methods for mediating gene suppression by using factors that enhance RNAi. Tuschl et al., International PCT Publication No. WO 02/44321, describe certain synthetic siRNA constructs. Pachuk et al., International PCT Publication No. WO 00/63364, and Satishchandran et al., International PCT Publication No. WO 01/04313, describe certain methods and compositions for inhibiting the function of certain polynucleotide sequences using certain dsRNAs. Echeverri et al., International PCT Publication No. WO 02/38805, describe certain C. elegans genes identified via RNAi. Kreutzer et al., International PCT Publications Nos. WO 02/055692, WO 02/055693, and EP 1144623 B1 describes certain methods for inhibiting gene expression using RNAi. Graham et al., International PCT Publications Nos. WO 99/49029 and WO 01/70949, and AU 4037501 describe certain vector expressed siRNA molecules. Fire et al., U.S. Pat. No. 6,506,559, describe certain methods for inhibiting gene expression in vitro using certain long dsRNA (greater than 25 nucleotide) constructs that mediate RNAi. Martinez et al., 2002, Cell, 110, 563-574, describe certain single stranded siRNA constructs, including certain 5′-phosphorylated single stranded siRNAs that mediate RNA interference in Hela cells. All of these references describe double stranded nucleic acid constructs where one of the two strands (the antisense strand) is complementary to the target RNA and the other strand (sense strand) is complementary to the antisense strand; the nucleotide sequence of the two strands are distinct and do not share sequence homology with each other. None of these references describe double stranded nucleic acid constructs where each strand of the double strand comprises nucleic acid sequence that is complementary to a target nucleic acid sequence and the nucleotide sequence of the two strands are homologus to each other.

### SUMMARY OF THE INVENTION

This invention relates to nucleic acid-based compounds, compositions, and methods useful for modulating RNA function and/or gene expression in a cell. Specifically, the instant invention features duplex forming oligonucleotides (DFO) that can self-assemble into double stranded oligonucleotides. The duplex forming oligonucleotides of the invention can be chemically synthesized or expressed from transcription units and/or vectors. The DFO molecules of the instant invention provide useful reagents and methods for a variety of therapeutic, diagnostic, agricultural, veterinary, target validation, genomic discovery, genetic engineering and pharmacogenomic applications.

Applicant demonstrates herein that certain oligonucleotides, refered to herein for convenience but not limitation as duplex forming oligonucleotides or DFO molecules, are potent mediators of sequence specific regulation of gene expression. The oligonucleotides of the invention are distinct from other nucleic acid sequences known in the art (e.g., siRNA, mRNA, stRNA, shRNA, antisense oligonucleotides etc.) in that they represent a class of linear polynucleotide sequences that are designed to self-assemble into double stranded oligonucleotides, where each strand in the double stranded oligonucleotides comprises nucleotide sequence that is complementary to a target nucleic acid molecule. Nucleic acid molecules of the invention can thus self assemble into functional duplexes in which each strand of the duplex comprises the same polynucleotide sequence and each strand comprises nucleotide sequence that is complementary to a target nucleic acid molecule.

Generally, double stranded oligonucleotides are formed by the assembly of two distinct oligonucleotide sequences where the oligonucleotide sequence of one strand is complementary to the oligonucleotide sequence of the second strand; such double stranded oligonucleotides are assembled from two separate oligonucleotides, or from a single molecule that folds on itself to form a double stranded structure, often referred to in the field as hairpin stem-loop structure (e.g. shRNA or short hairpin RNA). These double stranded oligonucleotides known in the art all have a common feature in that each strand of the duplex has a distict nucleotide sequence.

Distinct from the double stranded nucleic acid molecules known in the art, the applicants have developed a novel, potentially cost effective and simplified method of forming a double stranded nucleic acid molecule starting from a single stranded or linear oligonucleotide. The two strands of the double stranded oligonucleotide formed according to the instant invention have the same nucleotide sequence and are not covalently linked to each other. Such double-stranded oligonucleotides molecules can be readily linked post-synthetically by methods and reagents known in the art and are within the scope of the invention. In one embodiment, the single stranded oligonucleotide of the invention (the duplex forming oligonucleotide) that forms a double stranded oligonucleotide comprises a first region and a second region, where the second region includes nucleotide sequence that is an inverted repeat of the nucleotide sequence in the first region or a portion thereof, such that the single stranded oligonucleotide self assembles to form a duplex oligonucleotide in which the nucleotide sequence of one strand of the duplex is the same as the nucleotide sequence of the second strand. Non-limiting examples of such duplex forming oligonucleotides are illustrated in FIGS. 1 and 2. These duplex forming oligonucleotides (DFOs) can optionally include certain palindrome or repeat sequences where such palindrome or repeat sequences are present in between the first region and the second region of the DFO.

In one embodiment, the invention features a duplex forming oligonucleotide (DFO) molecule, wherein the DFO comprises a duplex forming self complementary nucleic acid sequence that has nucleotide sequence complementary to a target nucleic acid sequence. The DFO molecule can comprise a single self complementary sequence or a duplex resulting from assembly of such self complementary sequences.

In one embodiment, a duplex forming oligonucleotide (DFO) of the invention comprises a first region and a second region, wherein the second region comprises nucleotide sequence comprising an inverted repeat of nucleotide sequence of the first region, such that the DFO molecule can assemble into a double stranded oligonucleotide. Such double stranded oligonucleotides can act as a short interfering nucleic acid (siNA) to modulate gene expression. Each strand of the double stranded oligonucleotide duplex formed by DFO molecules of the invention can comprise a nucleotide sequence region that is complementary to the same nucleotide sequence in a target nucleic acid molecule (e.g., target RNA).

In one embodiment, the invention features a single stranded DFO that can assemble into a double stranded oligonucleotide. The applicant has surprisingly found that a single stranded oligonucleotide with nucleotide regions of self complementarity can readily assemble into duplex oligonucleotide constructs. Such DFOs can assemble into duplexes that can inhibit gene expression in a sequence specific manner. The DFO moleucles of the invention comprise a first region with nucleotide sequence that is complementary to the nucleotide sequence of a second region and where the sequence of the first region is complementary to a target nucleic acid (e.g., RNA). The DFO can form a double stranded oligonucleotide wherein a portion of each strand of the double stranded oligonucleotide comprises sequence complementary to a target nucleic acid sequence.

In one embodiment, the invention features a double stranded oligonucleotide, wherein the two strands of the double stranded oligonucleotide are not covalently linked to each other, and wherein each strand of the double stranded oligonucleotide comprises nucleotide sequence that is complementary to the same nucleotide sequence in a target nucleic acid molecule or a portion thereof. In another embodiment, the two strands of the double stranded oligonucleotide share an identical nucleotide sequence of at least about 15, preferably at least about 16, 17, 18, 19, 20, or 21 nucleotides.

In one embodiment, a DFO molecule of the invention comprises a structure having Formula I:
5′-p-XZX′-3′
wherein Z comprises a palindromic or repeat nucleic acid sequence or palindromic or repeat-like nucleic acid sequence with one or more modified nucleotides (e.g. nucleotide with a modified base, such as 2-amino purine, 2-amino-1,6-dihydro purine or a universal base), for example of length about 2 to about 24 nucleotides in even numbers (e.g. about 2, 4, 6, 8, 10, 12, 14, 16, 18, 20, or 22 or 24 nucleotides), X represents a nucleic acid sequence, for example of length between about 1 to about 21 nucleotides (e.g., about 1, 2, 3, 4, 5, 6, 7, 8, 9, 10, 11, 12, 13, 14, 15, 16, 17, 18, 19, 20, or 21 nucleotides), X′ comprises a nucleic acid sequence, for example of length about 1 and about 21 nucleotides (e.g., about 1, 2, 3, 4, 5, 6, 7, 8, 9, 10, 11, 12, 13, 14, 15, 16, 17, 18, 19, 20 or 21 nucleotides) having nucleotide sequence complementarity to sequence X or a portion thereof, p comprises a terminal phosphate group that can be present or absent, and wherein sequence X and Z, either independently or together, comprise nucleotide sequence that is complementary to a target nucleic acid sequence or a portion thereof and is of length sufficient to interact (e.g. base pair) with the target nucleic acid sequence of a portion thereof. For example, X independently can comprise sequence from about 12 to about 21 or more (e.g., about 12, 13, 14, 15, 16, 17, 18, 19, 20, 21, or more) nucleotides in length that is complementary to nucleotide sequence in a target RNA or a portion thereof. In another non-limiting example, the length of the nucleotide sequence of X and Z together, when X is present, that is complementary to the target RNA or a portion thereof is from about 12 to about 21 or more nucleotides (e.g., about 12, 13, 14, 15, 16, 17, 18, 19, 20, 21, or more). In yet another non-limiting example, when X is absent, the length of the nucleotide sequence of Z that is complementary to the target RNA or a portion thereof is from about 12 to about 24 or more nucleotides (e.g., about 12, 14, 16, 18, 20, 22, 24, or more). In one embodiment X, Z and X′ are independently oligonucleotides, where X and/or Z comprises nucleotide sequence of length sufficient to interact (e.g. base pair) with nucleotide sequence in the target RNA or a portion thereof. In one embodiment, the lengths of oligonucleotides X and X′ are identical. In another embodiment, the lengths of oligonucleotides X and X′ are not identical. In another embodiment, the lengths of oligonucleotides X and Z, or Z and X′, or X, Z and X′ are either identical or different.

In one embodiment, the invention features a double stranded oligonucleotide construct having Formula I(a):
5′-p-XZX′-3′
3′-X′ZX-p-5′
wherein Z comprises a palindromic or repeat nucleic acid sequence or palindromic or repeat-like nucleic acid sequence with one or more modified nucleotides (e.g. nucleotide with a modified base, such as 2-amino purine, 2-amino-1,6-dihydro purine or a universal base), for example of length about 2 to about 24 nucleotides in even numbers (e.g. about 2, 4, 6, 8, 10, 12, 14, 16, 18, 20, 22 or 24 nucleotides), X represents a nucleic acid sequence, for example of length about 1 to about 21 nucleotides (e.g., about 1, 2, 3, 4, 5, 6, 7, 8, 9, 10, 11, 12, 13, 14, 15, 16, 17, 18, 19, 20, or 21 nucleotides), X′ comprises a nucleic acid sequence, for example of length about 1 to about 21 nucleotides (e.g., about 1, 2, 3, 4, 5, 6, 7, 8, 9, 10, 11, 12, 13, 14, 15, 16, 17, 18, 19, 20 or 21 nucleotides) having nucleotide sequence complementarity to sequence X or a portion thereof, p comprises a terminal phosphate group that can be present or absent, and wherein each X and Z independently comprises nucleotide sequence that is complementary to a target nucleic acid sequence or a portion thereof and is of length sufficient to interact with the target nucleic acid sequence of a portion thereof. For example, sequence X independently can comprise sequence from about 12 to about 21 or more nucleotides (e.g., about 12, 13, 14, 15, 16, 17, 18, 19, 20, 21, or more) nucleotides in length that is complementary to nucleotide sequence in a target RNA or a portion thereof. In another non-limiting example, the length of the nucleotide sequence of X and Z together, when X is present, that is complementary to the target RNA or a portion thereof is from about 12 to about 21 or more nucleotides (e.g., about 12, 13, 14, 15, 16, 17, 18, 19, 20, 21, or more). In yet another non-limiting example, when X is absent, the length of the nucleotide sequence of Z that is complementary to the target RNA or a portion thereof is from about 12 to about 24 or more nucleotides (e.g., about 12, 14, 16, 18, 20, 22, 24 or more). In one embodiment X, Z and X′ are independently oligonucleotides, where X and/or Z comprises nucleotide sequence of length sufficient to interact (e.g. base pair) with nucleotide sequence in the target RNA or a portion thereof. In one embodiment, the lengths of oligonucleotides X and X′ are identical. In another embodiment, the lengths of oligonucleotides X and X′ are not identical. In another embodiment, the lengths of oligonucleotides X and Z or Z and X′ or X, Z and X′ are either identical or different. In one embodiment, the double stranded oligonucleotide construct of Formula I(a) includes one or more, specifically 1, 2, 3 or 4, mismatches, to the extent such mismatches do not significantly diminish the ability of the double stranded oligonucleotide to inhibit target gene expression.

In one embodiment, a DFO molecule of the invention comprises structure having Formula II:
5′-p-XX′-3′
wherein each X and X′ are independently oligonucleotides of length about 12 nucleotides to about 21 nucleotides, wherein X comprises, for example a nucleic acid sequence of length about 12 to about 21 nucleotides (e.g., about 12, 13, 14, 15, 16, 17, 18, 19, 20 or 21 nucleotides), X′ comprises a nucleic acid sequence, for example of length about 12 to about 21 nucleotides (e.g., about 12, 13, 14, 15, 16, 17, 18, 19, 20, or 21 nucleotides) having nucleotide sequence complementarity to sequence X or a portion thereof, p comprises a terminal phosphate group that can be present or absent, and wherein X comprises nucleotide sequence that is complementary to a target nucleic acid sequence (e.g. RNA) or a portion thereof and is of length sufficient to interact (e.g. base pair) with the target nucleic acid sequence of a portion thereof. In one embodiment, the length of oligonucleotides X and X′ are identical. In another embodiment the length of oligonucleotides X and X′ are not identical. In one embodiment, length of the oligonucleotides X and X′ are sufficient to form a relatively stable double stranded oligonucleotide.

In one embodiment, the invention features a double stranded oligonucleotide construct having Formula II(a):
5′-XX′-3′
3′-X′ X-p-5′
wherein each X and X′ are independently oligonucleotides of length about 12 nucleotides to about 21 nucleotides, wherein X comprises a nucleic acid sequence, for example of length about 12 to about 21 nucleotides (e.g., about 12, 13, 14, 15, 16, 17, 18, 19, 20 or 21 nucleotides), X′ comprises a nucleic acid sequence, for example of length about 12 to about 21 nucleotides (e.g., about 12, 13, 14, 15, 16, 17, 18, 19, 20 or 21 nucleotides) having nucleotide sequence complementarity to sequence X or a portion thereof, p comprises a terminal phosphate group that can be present or absent, and wherein X comprises nucleotide sequence that is complementary to a target nucleic acid sequence or a portion thereof and is of length sufficient to interact (e.g. base pair) with the target nucleic acid sequence (e.g. RNA) of a portion thereof. In one embodiment, the lengths of oligonucleotides X and X′ are identical. In another embodiment, the lengths of oligonucleotides X and X′ are not identical. In one embodiment, the lengths of the oligonucleotides X and X′ are sufficient to form a relatively stable double stranded oligonucleotide. In one embodiment, the double stranded oligonucleotide construct of Formula II(a) includes one or more, specifically 1, 2, 3 or 4, mismatches, to the extent such mismatches do not significantly diminish the ability of the double stranded oligonucleotide to inhibit target gene expression.

In one embodiment, the invention features a DFO molecule having Formula I(b):
5′-p-Z-3′
where Z comprises a palindromic or repeat nucleic acid sequence or palindromic or repeat like nucleic acid sequence with one or more non-standard or modified nucleotides (e.g. nucleotide with a modified base, such as 2-amino purine or a universal base) that can facilitate base-pairing with other nucleotides. Z can be for example of length sufficient to interact (e.g. base pair) with nucleotide sequence of a target nucleic acid (e.g. RNA) molecule, preferably of length of at least 12 nucleotides, specifically about 12 to about 24 nucleotides (e.g. about 12, 14, 16, 18, 20, 22 or 24 nucleotides). p represents a terminal phosphate group that can be present or absent.

In one embodiment, a DFO molecule having any of Formula I, I(a), I(b), II(a) or II can comprise chemical modifications as described herein without limitation, such as, for example, nucleotides having any of Formulae III-IX, stabilization chemistries as described in Table V, or any other combination of modified nucleotides and non-nucleotides as described in the various embodiments herein.

In one embodiment, the palidrome or repeat sequence or modified nucleotide (e.g. nucleotide with a modified base, such as 2-amino purine or a universal base) in Z of DFO constructs having Formula I, I(a) and I(b), comprises chemically modified nucleotides that are able to interact with a portion of the target nucleic acid sequence (e.g., modified base analogs that can form Watson Crick base pairs or non-Watson Crick base pairs).

In one embodiment, a DFO molecule of the invention, for example a DFO having Formula I or II, comprises about 15 to about 40 nucleotides (e.g., about 15, 16, 17, 18, 19, 20, 21, 22, 23, 24, 25, 26, 27, 28, 29, 30, 31, 32, 33, 34, 35, 36, 37, 38, 39, or 40 nucleotides). In one embodiment, a DFO molecule of the invention comprises one or more chemical modifications. In a non-limiting example, the introduction of chemically modified nucleotides and/or non-nucleotides into nucleic acid molecules of the invention provides a powerful tool in overcoming potential limitations of in vivo stability and bioavailability inherent to unmodified RNA molecules that are delivered exogenously. For example, the use of chemically modified nucleic acid molecules can enable a lower dose of a particular nucleic acid molecule for a given therapeutic effect since chemically modified nucleic acid molecules tend to have a longer half-life in serum or in cells or tissues. Furthermore, certain chemical modifications can improve the bioavailability and/or potency of nucleic acid molecules by not only enhancing half-life but also facilitating the targeting of nucleic acid molecules to particular organs, cells or tissues and/or improving cellular uptake of the nucleic acid molecules. Therefore, even if the activity of a chemically modified nucleic acid molecule is reduced in vitro as compared to a native/unmodified nucleic acid molecule, for example when compared to an unmodified RNA molecule, the overall activity of the modified nucleic acid molecule can be greater than the native or unmodified nucleic acid molecule due to improved stability, potency, duration of effect, bioavailability and/or delivery of the molecule.

In one embodiment, the invention features chemically modified DFO constructs having specificity for target nucleic acid molecules in a cell. Non-limiting examples of such chemical modifications independently include without limitation phosphate backbone modification (e.g. phosphorothioate internucleotide linkages), nucleotide sugar modification (e.g., 2′-O-methyl nucleotides, 2′-O-allyl nucleotides, 2′-deoxy-2′-fluoro nucleotides, 2′-deoxyribonucleotides), nucleotide base modification (e.g., “universal base” containing nucleotides, 5-C-methyl nucleotides), and non-nucleotide modification (e.g., abasic nucleotides, inverted deoxyabasic residue) or a combination of these modifications. These and other chemical modifications, when used in various DFO constructs, can preserve biological activity of the DFOs in vivo while at the same time, dramatically increasing the serum stability, potency, duration of effect and/or specificity of these compounds.

In one embodiment, a DFO molecule of the invention can generally comprise modified nucleotides from about 5 to about 100% of the nucleotide positions (e.g., 5%, 10%, 15%, 20%, 25%, 30%, 35%, 40%, 45%, 50%, 55%, 60%, 65%, 70%, 75%, 80%, 85%, 90%, 95% or 100% of the nucleotide positions may be modified). The actual percentage of modified nucleotides present in a given DFO molecule depends on the total number of nucleotides present in the DFO. If the DFO molecule is single stranded, the percent modification can be based upon the total number of nucleotides present in the single stranded DFO molecules. Likewise, if the DFO molecule is double stranded, the percent modification can be based upon the total number of nucleotides present in both strands. In addition, the actual percentage of modified nucleotides present in a given DFO molecule can also depend on the total number of purine and pyrimidine nucleotides present in the DFO, for example, wherein all pyrimidine nucleotides and/or all purine nucleotides present in the DFO molecule are modified.

In one embodiment, a DFO duplex molecule can comprise mismatches (e.g., 1, 2, 3 or 4 mismatches), bulges, loops, or wobble base pairs, for example, to modulate or regulate the ability of the DFO molecule to mediate inhibition of gene expression. Mismatches, bulges, loops, or wobble base pairs may be introduced into the DFO duplex molecules to the extent such mismatches, bulges, loops, or wobble base pairs do not significantly impair the ability of the DFOs to mediate inhibition of target gene expression. Such mismatches, bulges, loops, or wobble base pairs may be present in regions of the DFO duplex that do not significantly impair the ability of such DFOs to mediate inhibition of gene expression, for example, mismatches may be present at the terminal regions of the duplex or at one or positions in the internal regions of the duplex. Similarly, the wobble base pairs may, for example, be at the terminal base paired region(s) of the duplex or in the internal regions or in the regions where palindromic sequences are present withing the duplex oligonucleotide.

In one embodiment, a DFO molecule of the invention can comprise one or more (e.g., about 1, 2, 3, 4, or 5) phosphorothioate internucleotide linkages at the 3′-end of the DFO molecule.

In one embodiment, a DFO molecule of the invention comprises a 3′ nucleotide overhang region, which includes one or more (e.g., about 1, 2, 3, 4) unpaired nucleotides when the DFO is in duplex form. In a non-limiting example, the DFO duplex with overhangs includes a fewer number of base pairs than the number of nucleotides present in each strand of the DFO molecule (e.g., a DFO 18 nucleotides in length forming a 16 base-paired duplex with 2 nucleotide overhangs at the 3′ ends; see FIG. 1). Such blunt-end DFO duplex may optionally include one or more mismatches, wobble base-pairs or nucleotide bulges. The 3′-terminal nucleotide overhangs of a DFO molecule of the invention can comprise ribonucleotides or deoxyribonucleotides that are chemically-modified at a nucleic acid sugar, base, or phosphate backbone. The 3′-terminal nucleotide overhangs can comprise one or more universal base nucleotides. The 3′-terminal nucleotide overhangs can comprise one or more acyclic nucleotides or non-nucleotides.

In one embodiment, a DFO molecule of the invention in duplex form comprises blunt ends, i.e., the ends do not include any overhanging nucleotides. For example, a DFO duplex molecule of the invention comprising modifications described herein (e.g., comprising modifications having Formulae III-IX or DFO constructs comprising Stab1-Stab18 or any combination thereof) and/or any length described herein, has blunt ends or ends with no overhanging nucleotides.

In one embodiment, any DFO duplex of the invention can comprise one or more blunt ends, i.e. where a blunt end does not have any overhanging nucleotides. In a non-limiting example, a blunt ended DFO duplex includes the same number of base pairs as the number of nucleotides present in each strand of the DFO molecule (e.g., a DFO 18 nucleotides in length forming an 18 base-paired duplex; see FIG. 1). Such blunt-end DFO duplex may optionally include one or more mismatches, wobble base-pairs or nucleotide bulges.

By “blunt ends” is meant symmetric termini or termini of a DFO duplex having no overhanging nucleotides. The two strands of a DFO duplex molecule align with each other without over-hanging nucleotides at the termini (see FIG. 1). For example, a blunt ended DFO duplex comprises terminal nucleotides that are complementary between the two strands of the DFO duplex.

In one embodiment, the invention features a DFO molecule that down-regulates expression of a target gene in vitro or in vivo, wherein the DFO molecule comprises no ribonucleotides.

In one embodiment, a DFO molecule of the invention comprises sequence wherein one or more pyrimidine nucleotides present in the DFO sequence is a 2′-deoxy-2′-fluoro pyrimidine nucleotide. In another embodiment, a DFO molecule of the invention comprises sequence wherein all pyrimidine nucleotides present in the DFO sequence are 2′-deoxy-2′-fluoro pyrimidine nucleotides. Such DFO sequences can further comprise differing nucleotides or non-nucleotide caps described herein, such as deoxynucleotides, inverted nucleotides, abasic moieties, inverted abasic moieties, and/or any other modification shown in FIG. 9 or those modifications generally known in the art that can be introduced into nucleic acid molecules, to the extent any modification to the DFO molecule does not significantly impair the ability of the DFO molecule to mediate inhibition of gene expression.

In one embodiment, a DFO molecule of the invention comprises sequence wherein one or more purine nucleotides present in the DFO sequence is a 2′-sugar modified purine, (e.g., 2′-O-methyl purine nucleotide, 2′-O-allyl purine nucleotide, or 2′-methoxy-ethoxy purine nucleotides). In another embodiment, a DFO molecule of the invention comprises sequence wherein all purine nucleotides present in the DFO sequence are 2′-sugar modified purines, (e.g., 2′-O-methyl purine nucleotides, 2′-O-allyl purine nucleotides, or 2′-methoxy-ethoxy purine nucleotides).

In one embodiment, a DFO molecule of the invention comprises sequence wherein one or more purine nucleotides present in the DFO sequence is a 2′-deoxy purine nucleotide. In another embodiment, a DFO molecule of the invention comprises sequence wherein all purine nucleotides present in the DFO sequence are 2′-deoxy purine nucleotides.

In one embodiment, a DFO molecule of the invention comprises sequence wherein one or more purine nucleotides present in the DFO sequence is a 2′-deoxy-2′-fluoro purine nucleotide. In another embodiment, a DFO molecule of the invention comprises sequence wherein all purine nucleotides present in the DFO sequence are 2′-deoxy-2′-fluoro purine nucleotides.

In one embodiment, a DFO molecule of the invention comprises sequence wherein the DFO sequence includes a terminal cap moiety at the 5′-end, the 3′-end, or both of the 5′ and 3′ ends of the DFO sequence. In another embodiment, the terminal cap moiety is an inverted deoxy abasic moiety or any other modification shown in FIG. 8 or those modifications generally known in the art that can be introduced into nucleic acid molecules, to the extent any modification to the DFO molecule does not significantly impair the ability of the DFO molecule to mediate inhibition of gene expression.

In one embodiment, a DFO molecule of the invention comprises sequence wherein the DFO sequence includes a terminal cap moiety at the 3′ end of the DFO sequence. In another embodiment, the terminal cap moiety is an inverted deoxy abasic moiety or any other modification shown in FIG. 8 or those modifications generally known in the art that can be introduced into nucleic acid molecules, to the extent any modification to the DFO molecule does not significantly impair the ability of the DFO molecule to mediate inhibition of gene expression.

In one embodiment, a DFO molecule of the invention has activity that modulates expression of RNA encoded by a gene. Because many genes can share some degree of sequence homology with each other, DFO molecules can be designed to target a class of genes (and associated receptor or ligand genes) or alternately specific genes by selecting sequences that are either shared amongst different gene targets or alternatively that are unique for a specific gene target. Therefore, in one embodiment, the DFO molecule can be designed to target conserved regions of a RNA sequence having homology between several genes or genomes (e.g. viral genome, such as HIV, HCV, HBV, SARS and others) so as to target several genes or gene families (e.g., different gene isoforms, splice variants, mutant genes etc.) with one DFO molecule. In another embodiment, the DFO molecule can be designed to target a sequence that is unique to a specific RNA sequence of a specific gene or genome (e.g. viral genome, such as HIV, HCV, HBV, SARS and others). The expression of any target nucleic acid having known sequence can be modulated by DFO molecules of the invention (see for example McSwiggen et al., WO 03/74654 incorporated by reference herein in its entirety for a list of mammalian and viral targets).

In one embodiment, a DFO molecule of the invention does not contain any ribonucleotides. In another embodiment, a DFO molecule of the invention comprises one or more ribonucleotides.

In one embodiment, the DFO molecule of the invention does not include any chemical modification. In another embodiment, the DFO molecule of the invention is RNA comprising no chemical modifications. In another embodiment, the DFO molecule of the invention is RNA comprising two deoxyribonucleotides at the 3′-end. In another embodiment, the DFO molecule of the invention is RNA comprising a 3′-cap structure (e.g., inverted deoxynucleotide, inverted deoxy abasic moiety, a thymidine dinucleotide residues or a thymidine dinucleotide with a phosphorothioate internucleotide linkage, and the like).

In one embodiment of the present invention, each sequence of a DFO molecule is independently about 18 to about 300 nucleotides in length, in specific embodiments about 18-200 nucleotides in length, preferably 18-150 nucleotides in length, more specifically 18-100 nucleotides in length. In another embodiment, the DFO duplexes of the invention independently comprise about 18 to about 300 base pairs (e.g., about 18-200, 18-150, 18-100, 18-75, 18-50, 18-34 or 18-30 base pairs).

In one embodiment, the invention features a DFO molecule that inhibits the replication of a virus (e.g, as plant virus such as tobacco mosaic virus, or mammalian virus, such as hepatitis C virus, human immunodeficiency virus, hepatitis B virus, herpes simplex virus, cytomegalovirus, human papilloma virus, rhino virus, respiratory syncytial virus, SARS, or influenza virus).

In one embodiment, the invention features a medicament comprising a DFO molecule of the invention.

In one embodiment, the invention features an active ingredient comprising a DFO molecule of the invention.

In one embodiment, the invention features the use of a DFO molecule of the invention to down-regulate expression of a target gene.

In one embodiment, the invention features a composition comprising a DFO molecule of the invention and a pharmaceutically acceptable carrier or diluent.

In one embodiment, the invention features a method of increasing the stability of a DFO molecule against cleavage by ribonucleases or other nucleases, comprising introducing at least one modified nucleotide into the DFO molecule, wherein the modified nucleotide is for example a 2′-deoxy-2′-fluoro nucleotide. In another embodiment, all pyrimidine nucleotides present in the DFO are 2′-deoxy-2′-fluoro pyrimidine nucleotides. In another embodiment, the modified nucleotides in the DFO include at least one 2′-deoxy-2′-fluoro cytidine or 2′-deoxy-2′-fluoro uridine nucleotide. In another embodiment, the modified nucleotides in the DFO include at least one 2′-fluoro cytidine and at least one 2′-deoxy-2′-fluoro uridine nucleotides. In another embodiment, all uridine nucleotides present in the DFO are 2′-deoxy-2′-fluoro uridine nucleotides. In another embodiment, all cytidine nucleotides present in the DFO are 2′-deoxy-2′-fluoro cytidine nucleotides. In another embodiment, all adenosine nucleotides present in the DFO are 2′-deoxy-2′-fluoro adenosine nucleotides. In another embodiment, all guanosine nucleotides present in the DFO are 2′-deoxy-2′-fluoro guanosine nucleotides. The DFO can further comprise at least one modified internucleotidic linkage, such as phosphorothioate linkage or phosphorodithioate linkage. In another embodiment, the 2′-deoxy-2′-fluoronucleotides are present at specifically selected locations in the DFO that are sensitive to cleavage by ribonucleases or other nucleases, such as locations having pyrimidine nucleotides or terminal nucleotides. The DFO molecules of the invention can be modified to improve stability, pharmacokinetic properties, in vitro or in vivo delivery, localization and/or potency by methods generally known in the art (see for example Beigelman et al., WO WO 03/70918 incorporated by reference herein in its entirety including the drawings).

In one embodiment, a DFO molecule of the invention comprises nucleotide sequence having complementarity to nucleotide sequence of RNA or a portion thereof encoded by the target nucleic acid or a portion thereof.

In one embodiment, the invention features a DFO molecule having a first region and a second region, wherein the second region comprises nucleotide sequence that is an inverted repeat sequence of the nucleotide sequence of the first region, wherein the first region is complementary to nucleotide sequence of a target nucleic acid (e.g., RNA) or a portion thereof (see for example FIGS. 1 and 2 for an illustration of non-limiting examples of DFO molecules of the instant inventon).

One embodiment of the invention provides an expression vector comprising a nucleic acid sequence encoding at least one DFO molecule of the invention in a manner that allows expression of the DFO sequence. Another embodiment of the invention provides a mammalian cell comprising such an expression vector. The mammalian cell can be a human cell.

In one embodiment, a DFO molecule of the invention comprises one or more (e.g., about 1, 2, 3, 4, 5, 6, 7, 8, 9, 10, or more) nucleotides comprising a backbone modified internucleotide linkage having Formula III: 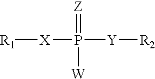
wherein each R1 and R2 is independently any nucleotide, non-nucleotide, or polynucleotide which can be naturally-occurring or chemically-modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y, and Z are optionally not all O. In another embodiment, a backbone modification of the invention comprises a phosphonoacetate and/or thiophosphonoacetate internucleotide linkage (see for example Sheehan et al., 2003, Nucleic Acids Research, 31, 4109-4118).

The chemically-modified internucleotide linkages having Formula III, for example, wherein any Z, W, X, and/or Y independently comprises a sulphur atom, can be present anywhere in the DFO sequence. Non-limiting examples of such phosphate backbone modifications are phosphorothioate and phosphorodithioate. The DFO molecules of the invention can comprise one or more (e.g., about 1, 2, 3, 4, 5, 6, 7, 8, 9, 10, or more) chemically-modified internucleotide linkages having Formula III at the 3′-end, the 5′-end, or both of the 3′ and 5′-ends of the DFO sequence. In another non-limiting example, an exemplary DFO molecule of the invention can comprise one or more (e.g., about 1, 2, 3, 4, 5, 6, 7, 8, 9, 10, or more) pyrimidine nucleotides with chemically-modified internucleotide linkages having Formula III. In yet another non-limiting example, an exemplary DFO molecule of the invention can comprise one or more (e.g., about 1, 2, 3, 4, 5, 6, 7, 8, 9, 10, or more) purine nucleotides with chemically-modified internucleotide linkages having Formula III. In another embodiment, a DFO molecule of the invention having internucleotide linkage(s) of Formula III also comprises a chemically-modified nucleotide or non-nucleotide having any of Formulae III-IX.

In one embodiment, a DFO molecule of the invention comprises one or more (e.g., about 1, 2, 3, 4, 5, 6, 7, 8, 9, 10, or more) nucleotides or non-nucleotides having Formula IV: 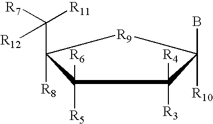
wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO₂, NO₂, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula III or IV; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, 2-aminopurine, 2-amino-1,6-dihydropurine or any other non-naturally occurring base that can be complementary or non-complementary to target RNA or a non-nucleosidic base such as phenyl, naphthyl, 3-nitropyrrole, 5-nitroindole, nebularine, pyridone, pyridinone, or any other non-naturally occurring universal base that can be complementary or non-complementary to target RNA.

The chemically-modified nucleotide or non-nucleotide of Formula IV can be present anywhere in the DFO sequence. The DFO molecules of the invention can comprise one or more chemically-modified nucleotide or non-nucleotide of Formula IV at the 3′-end, the 5′-end, or both of the 3′ and 5′-ends of the DFO sequence. For example, an exemplary DFO molecule of the invention can comprise about 1 to about 5 or more (e.g., about 1, 2, 3, 4, 5, or more) chemically-modified nucleotides or non-nucleotides of Formula IV at the 5′-end of the DFO sequence. In another non-limiting example, an exemplary DFO molecule of the invention can comprise about 1 to about 5 or more (e.g., about 1, 2, 3, 4, 5, or more) chemically-modified nucleotides or non-nucleotides of Formula IV at the 3′-end of the DFO sequence.

In one embodiment, a DFO molecule of the invention comprises one or more (e.g., about 1, 2, 3, 4, 5, 6, 7, 8, 9, 10, or more) nucleotides or non-nucleotides having Formula V: 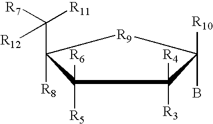
wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO₂, NO₂, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula III or IV; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base that can be employed to be complementary or non-complementary to target RNA or a non-nucleosidic base such as phenyl, naphthyl, 3-nitropyrrole, 5-nitroindole, nebularine, pyridone, pyridinone, or any other non-naturally occurring universal base that can be complementary or non-complementary to target RNA.

The chemically-modified nucleotide or non-nucleotide of Formula V can be present anywhere in the DFO sequence. The DFO molecules of the invention can comprise one or more chemically-modified nucleotide or non-nucleotide of Formula V at the 3′-end, the 5′-end, or both of the 3′ and 5′-ends of the DFO sequence. For example, an exemplary DFO molecule of the invention can comprise about 1 to about 5 or more (e.g., about 1, 2, 3, 4, 5, or more) chemically-modified nucleotide(s) or non-nucleotide(s) of Formula V at the 5′-end of DFO sequence. In anther non-limiting example, an exemplary DFO molecule of the invention can comprise about 1 to about 5 or more (e.g., about 1, 2, 3, 4, 5, or more) chemically-modified nucleotide or non-nucleotide of Formula V at the 3′-end of the DFO sequence.

In another embodiment, a DFO molecule of the invention comprises a nucleotide having Formula IV or V, wherein the nucleotide having Formula IV or V is in an inverted configuration. For example, the nucleotide having Formula IV or V is connected to the DFO construct in a 3′-3′,3′-2′,2′-3′, or 5′-5′ configuration, such as at the 3′-end, the 5′-end, or both of the 3′ and 5′-ends of one or both DFO strands.

In one embodiment, a DFO molecule of the invention comprises a 5′-terminal phosphate group having Formula VI: 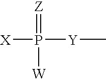
wherein each X and Y is independently O, S, N, alkyl, substituted alkyl, or alkylhalo; wherein each Z and W is independently O, S, N, alkyl, substituted alkyl, O-alkyl, S-alkyl, alkaryl, aralkyl, or alkylhalo or acetyl; and/or wherein W, X, Y and Z are optionally not all O.

In another embodiment, a DFO molecule of the invention comprises one or more (e.g., 1, 2, 3, 4, 5, 6, 7, 8, 9, 10 or more) 2′-5′ internucleotide linkages. The 2′-5′ internucleotide linkage(s) can be anywhere in the DFO sequence. In addition, the 2′-5′ internucleotide linkage(s) can be present at various other positions within the DFO sequence, for example, about 1, 2, 3, 4, 5, 6, 7, 8, 9, 10, or more including every internucleotide linkage of a pyrimidine nucleotide in the DFO molecule can comprise a 2′-5′ internucleotide linkage, or about 1, 2, 3, 4, 5, 6, 7, 8, 9, 10, or more including every internucleotide linkage of a purine nucleotide in the DFO molecule can comprise a 2′-5′ internucleotide linkage.

In one embodiment, a DFO molecule of the invention comprises at least one (e.g., about 1, 2, 3, 4, 5, 6, 7, 8, 9, 10, or more) abasic moiety, for example a compound having Formula VII: 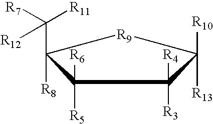
wherein each R3, R4, R5, R6, R7, R8, R10, R11, R12, and R13 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO₂, NO₂, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula III or IV; R9 is O, S, CH2, S═O, CHF, or CF2.

In one embodiment, a DFO molecule of the invention comprises at least one (e.g., about 1, 2, 3, 4, 5, 6, 7, 8, 9, 10, or more) inverted nucleotide or abasic moiety, for example a compound having Formula VIII: 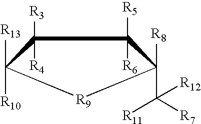
wherein each R3, R4, R5, R6, R7, R8, R10, R11, R12, and R13 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO₂, NO₂, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula III or IV; R9 is O, S, CH2, S═O, CHF, or CF2, and either R3, R5, R8 or R13 serve as points of attachment to the DFO molecule of the invention.

In another embodiment, a DFO molecule of the invention comprises at least one (e.g., about 1, 2, 3, 4, 5, 6, 7, 8, 9, 10, or more) substituted polyalkyl moieties, for example a compound having Formula IX: 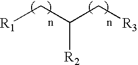
wherein each n is independently an integer from 1 to 12, each R1, R2 and R3 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO₂, NO₂, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or a group having Formula III, and R1, R2 or R3 serves as points of attachment to the DFO molecule of the invention.

In another embodiment, the invention features a compound having Formula IX, wherein R1 and R2 are hydroxyl (OH) groups, n=1, and R3 comprises O and is the point of attachment to the 3′-end, the 5′-end, or both of the 3′ and 5′-ends of one or both strands of a DFO molecule of the invention. This modification is referred to herein as “glyceryl” (for example modification 6 in FIG. 9).

In another embodiment, a moiety having any of Formula VII, VIII or IX of the invention is at the 3′-end, the 5′-end, or both of the 3′ and 5′-ends of a DFO molecule of the invention. In another embodiment, a moiety having any of Formula VII, VIII or IX of the invention is at the 3′-end of a DFO molecule of the invention.

In another embodiment, a DFO molecule of the invention comprises an abasic residue having Formula VII or VIII, wherein the abasic residue having Formula VII or VIII is connected to the DFO construct in a 3-3′, 3-2′, 2-3′, or 5-5′ configuration, such as at the 3′-end, the 5′-end, or both of the 3′ and 5′-ends of the DFO molecule. In another embodiment, a DFO molecule of the invention comprises an abasic residue having Formula VII or VIII, wherein the abasic residue having Formula VII or VIII is connected to the DFO construct in a 3-3′ or 3-2′ configuration at the 3′-end of the DFO molecule.

In one embodiment, a DFO molecule of the invention comprises one or more (e.g., about 1, 2, 3, 4, 5, 6, 7, 8, 9, 10, or more) locked nucleic acid (LNA) nucleotides, for example at the 5′-end, the 3′-end, both of the 5′ and 3′-ends, or any combination thereof, of the DFO molecule.

In another embodiment, a DFO molecule of the invention comprises one or more (e.g., about 1, 2, 3, 4, 5, 6, 7, 8, 9, 10, or more) acyclic nucleotides, for example at the 5′-end, the 3′-end, both of the 5′ and 3′-ends, or any combination thereof, of the DFO molecule. In another embodiment, a DFO molecule of the invention comprises one or more (e.g., about 1, 2, 3, 4, 5, 6, 7, 8, 9, 10, or more) acyclic nucleotides at the 3′-end of the DFO molecule.

In one embodiment, a DFO molecule of the invention comprises a terminal cap moiety, (see for example FIG. 8) such as an inverted deoxyabasic moiety or inverted nucleotide, at the 3′-end, 5′-end, or both 3′ and 5′-ends of the DFO molecule. In another embodiment, a DFO molecule of the invention comprises a terminal cap moiety, (see for example FIG. 8) such as an inverted deoxyabasic moiety or inverted nucleotide, at the 3′-end of the DFO molecule.

In one embodiment, a DFO molecule of the invention comprises sequence wherein any (e.g., one or more or all) pyrimidine nucleotides present in the DFO are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g., one or more or all) purine nucleotides present in the DFO are 2′-deoxy purine nucleotides (e.g., wherein all purine nucleotides are 2′-deoxy purine nucleotides or alternately a plurality of purine nucleotides are 2′-deoxy purine nucleotides). The DFO can further comprise terminal cap modifications as described herein.

In one embodiment, a DFO molecule of the invention comprises sequence wherein any (e.g., one or more or all) pyrimidine nucleotides present in the DFO are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g., one or more or all) purine nucleotides present in the DFO are 2′-O-methyl purine nucleotides (e.g., wherein all purine nucleotides are 2′-O-methyl purine nucleotides or alternately a plurality of purine nucleotides are 2′-O-methyl purine nucleotides). The DFO can further comprise terminal cap modifications as described herein.

In one embodiment, a DFO molecule of the invention comprises sequence wherein any (e.g., one or more or all) pyrimidine nucleotides present in the DFO are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any (e.g., one or more or all) purine nucleotides present in the DFO are selected from the group consisting of 2′-deoxy nucleotides, locked nucleic acid (LNA) nucleotides, 2′-methoxyethyl nucleotides, 4′-thionucleotides, and 2′-O-methyl nucleotides (e.g., wherein all purine nucleotides are selected from the group consisting of 2′-deoxy nucleotides, locked nucleic acid (LNA) nucleotides, 2′-methoxyethyl nucleotides, 4′-thionucleotides, and 2′-O-methyl nucleotides or alternately a plurality of purine nucleotides are selected from the group consisting of 2′-deoxy nucleotides, locked nucleic acid (LNA) nucleotides, 2′-methoxyethyl nucleotides, 4′-thionucleotides, and 2′-O-methyl nucleotides).

In another embodiment, a DFO molecule of the invention comprises modified nucleotides having properties or characteristics similar to naturally occurring ribonucleotides. For example, the invention features DFO molecules including modified nucleotides having a Northern conformation (e.g., Northern pseudorotation cycle, see for example Saenger, Principles of Nucleic Acid Structure, Springer-Verlag ed., 1984). As such, chemically modified nucleotides present in the DFO molecules of the invention are resistant to nuclease degradation while at the same time maintaining the capacity to modulate gene expression. Non-limiting examples of nucleotides having a northern configuration include locked nucleic acid (LNA) nucleotides (e.g., 2′-O,4′-C-methylene-(D-ribofuranosyl) nucleotides); 2′-methoxyethoxy (MOE) nucleotides; 2′-methyl-thio-ethyl, 2′-deoxy-2′-fluoro nucleotides, 2′-deoxy-2′-chloro nucleotides, 2′-azido nucleotides, and 2′-O-methyl nucleotides.

In one embodiment, a DFO molecule of the invention comprises a conjugate attached to the DFO molecule. For example, the conjugate can be attached to the DFO molecule via a covalent attachment. In one embodiment, the conjugate is attached to the DFO molecule via a biodegradable linker. In one embodiment, the conjugate molecule is attached at the 3′-end of the DFO molecule. In another embodiment, the conjugate molecule is attached at the 5′-end of the DFO molecule. In yet another embodiment, the conjugate molecule is attached at both the 3′-end and 5′-end of the DFO molecule, or any combination thereof. In one embodiment, the conjugate molecule of the invention comprises a molecule that facilitates delivery of a DFO molecule into a biological system, such as a cell. In another embodiment, the conjugate molecule attached to the chemically-modified DFO molecule is a polyethylene glycol, human serum albumin, or a ligand for a cellular receptor that can mediate cellular uptake. Examples of specific conjugate molecules contemplated by the instant invention that can be attached to DFO molecules are described in Vargeese et al., U.S. Ser. No. 10/201,394, incorporated by reference herein. The type of conjugates used and the extent of conjugation of DFO molecules of the invention can be evaluated for improved pharmacokinetic profiles, bioavailability, and/or stability of DFO constructs while at the same time maintaining the ability of the DFO to modulate gene expression. As such, one skilled in the art can screen DFO constructs that are modified with various conjugates to determine whether the DFO conjugate complex possesses improved properties while maintaining the ability to modulate gene expression, for example in animal models as are generally known in the art.

In one embodiment, a DFO molecule of the invention comprises a non-nucleotide linker, such as an abasic nucleotide, polyether, polyamine, polyamide, peptide, carbohydrate, lipid, polyhydrocarbon, or other polymeric compounds (e.g. polyethylene glycols such as those having between 2 and 100 ethylene glycol units). Specific examples include those described by Seela and Kaiser, Nucleic Acids Res. 1990, 18:6353 and Nucleic Acids Res. 1987, 15:3113; Cload and Schepartz, J. Am. Chem. Soc: 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine, for example at the Cl position of the sugar.

In one embodiment, the invention features a DFO molecule that does not require the presence of a 2′-OH group (ribonucleotide) to be present within the DFO molecule to support inhibition or modulation of gene expression of a target nucleic acid.

In one embodiment, the invention features a method for modulating the expression of a gene within a cell comprising: (a) synthesizing a DFO molecule of the invention, which can be chemically-modified or unmodified, wherein the DFO comprises sequence complementary to RNA of the gene or a portion thereof; and (b) introducing the DFO molecule into a cell under conditions suitable to modulate the expression of the gene in the cell.

In another embodiment, the invention features a method for modulating the expression of more than one gene within a cell comprising: (a) synthesizing one or more DFO molecules of the invention, which can be chemically-modified or unmodified, wherein the DFO comprises sequence complementary to RNA of the genes or a portion thereof; and (b) introducing the DFO molecule(s) into a cell under conditions suitable to modulate the expression of the genes in the cell.

In one embodiment, DFO molecules of the invention are used as reagents in ex vivo applications. For example, DFO reagents are intoduced into tissue or cells that are transplanted into a subject for therapeutic effect. The cells and/or tissue can be derived from an organism or subject that later receives the explant, or can be derived from another organism or subject prior to transplantation. The DFO molecules can be used to modulate the expression of one or more genes in the cells or tissue, such that the cells or tissue obtain a desired phenotype or are able to perform a function when transplanted in vivo. In one embodiment, certain target cells from a patient are extracted. These extracted cells are contacted with DFOs targeting a specific nucleotide sequence within the cells under conditions suitable for uptake of the DFOs by these cells (e.g., using delivery reagents such as cationic lipids, liposomes and the like or using techniques such as electroporation to facilitate the delivery of DFOs into cells). The cells are then reintroduced back into the same patient or other patients. Non-limiting examples of ex vivo applications include use in organ/tissue transplant, tissue grafting, or treatment of pulmonary disease (e.g., restenosis) or prevent neointimal hyperplasia and atherosclerosis in vein grafts. Such ex vivo applications may also be used to treat conditions associated with coronary and peripheral bypass graft failure, for example, such methods can be used in conjunction with peripheral vascular bypass graft surgery and coronary artery bypass graft surgery. Additional applications include transplants to treat CNS lesions or injury, including use in treatment of neurodegenerative conditions such as Alzheimer's disease, Parkinson's Disease, Epilepsy, Dementia, Huntington's disease, or amyotrophic lateral sclerosis (ALS).

In one embodiment, the invention features a method of modulating the expression of a gene in a tissue explant comprising: (a) synthesizing a DFO molecule of the invention, which can be chemically-modified or unmodified, wherein the DFO comprises sequence complementary to RNA of the gene or a portion thereof, and (b) introducing the DFO molecule into a cell of the tissue explant derived from a particular organism under conditions suitable to modulate the expression of the gene in the tissue explant. In another embodiment, the method further comprises introducing the tissue explant back into the organism the tissue was derived from or into another organism under conditions suitable to modulate the expression of the gene in that organism.

In one embodiment, the invention features a method of modulating the expression of a gene in a tissue explant comprising: (a) synthesizing a DFO molecule of the invention, which can be chemically-modified or unmodified, wherein the DFO comprises sequence complementary to RNA of the gene or a portion thereof and wherein the sense strand sequence of the DFO comprises a sequence substantially similar to the sequence of the target RNA; and (b) introducing the DFO molecule into a cell of the tissue explant derived from a particular organism under conditions suitable to modulate the expression of the gene in the tissue explant. In another embodiment, the method further comprises introducing the tissue explant back into the organism the tissue was derived from or into another organism under conditions suitable to modulate the expression of the gene in that organism.

In another embodiment, the invention features a method of modulating the expression of more than one gene in a tissue explant comprising: (a) synthesizing one or more DFO molecules of the invention, which can be chemically-modified or unmodified, wherein the DFO comprise sequence complementary to RNA of the genes or a portion thereof; and (b) introducing the DFO molecule(s) into a cell of the tissue explant derived from a particular organism under conditions suitable to modulate the expression of the genes in the tissue explant. In another embodiment, the method further comprises introducing the tissue explant back into the organism the tissue was derived from or into another organism under conditions suitable to modulate the expression of the genes in that organism.

In one embodiment, the invention features a method of modulating the expression of a gene in an organism comprising: (a) synthesizing a DFO molecule of the invention, which can be chemically-modified or unmodified, wherein one of the DFO strands comprises a sequence complementary to RNA of the gene or a portion thereof; and (b) introducing the DFO molecule into the organism under conditions suitable to modulate the expression of the gene in the organism.

In another embodiment, the invention features a method of modulating the expression of more than one gene in an organism comprising: (a) synthesizing one or more DFO molecules of the invention, which can be chemically-modified or unmodified, wherein the DFO comprises sequence complementary to RNA of the genes or a portion thereof; and (b) introducing the DFO molecule(s) into the organism under conditions suitable to modulate the expression of the genes in the organism.

In one embodiment, the invention features a method of modulating the expression of a target gene in an tissue or organ comprising: (a) synthesizing a DFO molecule of the invention, which can be chemically-modified or unmodified, wherein the DFO comprises sequence having complementarity to RNA of the target gene; and (b) introducing the DFO molecule into the tissue or organ under conditions suitable to modulate the expression of the target gene in the organism. In another embodiment, the tissue is ocular tissue and the organ is the eye. In another embodiment, the tissue comprises hepatocytes and/or hepatic tissue and the organ is the liver.

In one embodiment, the invention features a method of modulating the expression of a target gene in an tissue or organ comprising: (a) synthesizing a DFO molecule of the invention, which can be chemically-modified or unmodified, wherein the DFO comprises a single stranded sequence having complementarity to RNA of the target gene; and (b) introducing the DFO molecule into the tissue or organ under conditions suitable to modulate the expression of the target gene in the organism. In another embodiment, the tissue is ocular tissue and the organ is the eye. In another embodiment, the tissue comprises hepatocytes and/or hepatic tissue and the organ is the liver.

In one embodiment, the invention features a method of modulating the expression of a gene in an organism comprising contacting the organism with a DFO molecule of the invention under conditions suitable to modulate the expression of the gene in the organism.

In another embodiment, the invention features a method of modulating the expression of more than one gene in an organism comprising contacting the organism with one or more DFO molecules of the invention under conditions suitable to modulate the expression of the genes in the organism.

The DFO molecules of the invention can be designed to down regulate or inhibit target gene expression in a biological system by targeting of a variety of RNA molecules. In one embodiment, the DFO molecules of the invention are used to target various RNAs corresponding to a target gene. Non-limiting examples of such RNAs include messenger RNA (mRNA), alternate RNA splice variants of target gene(s), post-transcriptionally modified RNA of target gene(s), pre-mRNA of target gene(s), and/or RNA templates. If alternate splicing produces a family of transcripts that are distinguished by usage of appropriate exons, the instant invention can be used to inhibit gene expression through the appropriate exons to specifically inhibit or to distinguish among the functions of gene family members. For example, a protein that contains an alternatively spliced transmembrane domain can be expressed in both membrane bound and secreted forms. Use of the invention to target the exon containing the transmembrane domain can be used to determine the functional consequences of pharmaceutical targeting of membrane bound as opposed to the secreted form of the protein. Non-limiting examples of applications of the invention relating to targeting these RNA molecules include therapeutic pharmaceutical applications, pharmaceutical discovery applications, molecular diagnostic and gene function applications, and gene mapping, for example using single nucleotide polymorphism mapping with DFO molecules of the invention. Such applications can be implemented using known gene sequences or from partial sequences available from an expressed sequence tag (EST).

In another embodiment, the DFO molecules of the invention are used to target conserved sequences corresponding to a gene family or gene families. As such, DFO molecules targeting multiple gene targets can provide increased therapeutic effect. In addition, DFO can be used to characterize pathways of gene function in a variety of applications. For example, the present invention can be used to inhibit the activity of target gene(s) in a pathway to determine the function of uncharacterized gene(s) in gene function analysis, mRNA function analysis, or translational analysis. The invention can be used to determine potential target gene pathways involved in various diseases and conditions toward pharmaceutical development. The invention can be used to understand pathways of gene expression involved in, for example, in development, such as prenatal development and postnatal development, and/or the progression and/or maintenance of cancer, infectious disease, autoimmunity, inflammation, endocrine disorders, renal disease, ocular disease, pulmonary disease, neurologic disease, cardiovascular disease, birth defects, aging, any other disease or condition related to gene expression.

In one embodiment, DFO molecule(s) and/or methods of the invention are used to down-regulate or inhibit the expression of gene(s) that encode RNA referred to by Genbank Accession, for example genes encoding RNA sequence(s) referred to herein by Genbank Accession number. See, for example, McSwiggen et al., WO 03/74654 incorporated by reference herein in its entirety for a list of mammalian and viral targets.

In one embodiment, the invention features a method comprising: (a) generating a library of DFO constructs having a predetermined complexity; and (b) assaying the DFO constructs of (a) above, under conditions suitable to determine accessible target sites within the target RNA sequence. In one embodiment, the DFO molecules of (a) have strands of a fixed length, for example, about 28 nucleotides in length. In another embodiment, the DFO molecules of (a) are of differing length, for example having strands of about 19 to about 34 (e.g., about 19, 20, 21, 22, 23, 24, 25, 26, 27, 28, 29, 30, 31, 32, 33, or 34) nucleotides in length. The assay can comprise a cell culture system in which target RNA is expressed. In another embodiment, fragments of target RNA are analyzed for detectable levels of cleavage, for example by gel electrophoresis, northern blot analysis, or RNAse protection assays, to determine the most suitable target site(s) within the target RNA sequence. The target RNA sequence can be obtained as is known in the art, for example, by cloning and/or transcription for in vitro systems, and by cellular expression in in vivo systems.

By “target site” is meant a sequence within a target RNA that is “targeted” for cleavage mediated by a DFO construct which contains sequences within its antisense region that are complementary to the target sequence.

By “detectable level of cleavage” is meant cleavage of target RNA (and formation of cleaved product RNAs) to an extent sufficient to discern cleavage products above the background of RNAs produced by random degradation of the target RNA. Production of cleavage products from 1-5% of the target RNA is sufficient to detect above the background for most methods of detection.

In one embodiment, the invention features a composition comprising a DFO molecule of the invention, which can be chemically-modified or ummodified, in a pharmaceutically acceptable carrier or diluent. In another embodiment, the invention features a pharmaceutical composition comprising DFO molecules of the invention, which can be chemically-modified, targeting one or more genes in a pharmaceutically acceptable carrier or diluent. In another embodiment, the invention features a method for diagnosing a disease or condition in a subject comprising administering to the subject a composition of the invention under conditions suitable for the diagnosis of the disease or condition in the subject. In another embodiment, the invention features a method for treating or preventing a disease or condition in a subject, comprising administering to the subject a composition of the invention under conditions suitable for the treatment or prevention of the disease or condition in the subject, alone or in conjunction with one or more other therapeutic compounds. In yet another embodiment, the invention features a method for reducing or preventing tissue rejection in a subject comprising administering to the subject a composition of the invention under conditions suitable for the reduction or prevention of tissue rejection in the subject.

In another embodiment, the invention features a method for validating a gene target in a biological system comprising: (a) synthesizing a DFO molecule of the invention, which can be chemically-modified or unmodified, wherein the DFO comprises a sequence complementary to RNA of a target gene or a portion thereof; (b) introducing the DFO molecule into a cell, tissue, or organism under conditions suitable for modulating expression of the target gene in the cell, tissue, or organism; and (c) determining the function of the gene by assaying for any phenotypic change in the cell, tissue, or organism.

In another embodiment, the invention features a method for validating a target gene comprising: (a) synthesizing a DFO molecule of the invention, which can be chemically-modified or unmodified, wherein the DFO strands includes a sequence complementary to RNA of a target gene or a portion thereof; (b) introducing the DFO molecule into a biological system under conditions suitable for modulating expression of the target gene in the biological system; and (c) determining the function of the gene by assaying for any phenotypic change in the biological system.

By “biological system” is meant, material, in a purified or unpurified form, from biological sources, including but not limited to human, animal, plant, insect, bacterial, viral or other sources, wherein the system comprises the components required for biologic acitivity (e.g., inhibition of gene expression). The term “biological system” includes, for example, a cell, tissue, or organism, or extract thereof.

By “phenotypic change” is meant any detectable change to a cell that occurs in response to contact or treatment with a nucleic acid molecule of the invention (e.g., DFO). Such detectable changes include, but are not limited to, changes in shape, size, proliferation, motility, protein expression or RNA expression or other physical or chemical changes as can be assayed by methods known in the art. The detectable change can also include expression of reporter genes/molecules such as Green Florescent Protein (GFP) or various tags that are used to identify an expressed protein or any other cellular component that can be assayed.

In one embodiment, the invention features a kit containing a DFO molecule of the invention, which can be chemically-modified or unmodified, that can be used to modulate the expression of a target gene in biological system, including, for example, in a cell, tissue, or organism. In another embodiment, the invention features a kit containing more than one DFO molecule of the invention, which can be chemically-modified, that can be used to modulate the expression of more than one target gene in a biological system, including, for example, in a cell, tissue, or organism.

In one embodiment, the invention features a kit containing a DFO molecule of the invention, which can be chemically-modified or unmodified, that can be used to modulate the expression of a target gene in a biological system. In another embodiment, the invention features a kit containing more than one DFO molecule of the invention, which can be chemically-modified, that can be used to modulate the expression of more than one target gene in a biological system.

In one embodiment, the invention features a cell containing one or more DFO molecules of the invention, which can be chemically-modified or unmodified. In another embodiment, the cell containing a DFO molecule of the invention is a mammalian cell. In yet another embodiment, the cell containing a DFO molecule of the invention is a human cell.

In one embodiment, the synthesis of a DFO duplex molecule of the invention, which can be chemically-modified or unmodified, comprises: (a) synthesizing a self complementary nucleic acid sequence comprising nucleic acid molecule, defined herein as DFO molecule; (b) incubating the nucleic acid molecule of (a) under conditions suitable for the DFO molecule to form a double-stranded DFO molecule. In one embodiment, synthesis of the self complementary nucleic acid sequence containing oligonucleotide or DFO is by solid phase oligonucleotide synthesis. In another embodiment the DFO molecule is expressed from an expression vector or is enzymatically synthesized.

In one embodiment, the synthesis of a DFO duplex molecule of the invention, which can be chemically-modified or unmodified, comprises: (a) synthesizing a nucleic acid molecule, wherein a first region comprises nucleotide sequence that is complementary to a target RNA or a portion thereof and is an inverted repeat of nucleotide sequence in the second region of the nucleic acid molecule, defined herein as the DFO molecule; (b) incubating the nucleic acid molecule of (a) under conditions suitable for the DFO molecule to form a double-stranded DFO molecule. In one embodiment, synthesis of the DFO molecule is by solid phase oligonucleotide synthesis. In another embodiment the DFO molecule is expressed from an expression vector or is enzymatically synthesized.

In another embodiment, the method of synthesis of DFO molecules of the invention comprises the teachings of Scaringe et al., U.S. Pat. Nos. 5,889,136; 6,008,400; and 6,111,086, incorporated by reference herein in their entirety.

In one embodiment, the invention features a DFO construct that mediates modulation or inhibition of gene expression in a cell or reconstituted system, wherein the DFO construct comprises one or more chemical modifications, for example, one or more chemical modifications having any of Formulae III-IX or any combination thereof that increases the nuclease resistance and/or overall effectiveness or potency of the DFO construct.

In another embodiment, the invention features a method for generating DFO molecules with increased nuclease resistance comprising (a) introducing nucleotides having any of Formula III-IX or any combination thereof into a DFO molecule, and (b) assaying the DFO molecule of step (a) under conditions suitable for isolating DFO molecules having increased nuclease resistance.

In another embodiment, the invention features a method for generating DFO molecules with increased duration of effect comprising (a) introducing nucleotides having any of Formula III-IX or any combination thereof into a DFO molecule, and (b) assaying the DFO molecule of step (a) under conditions suitable for isolating DFO molecules having increased duration of effect.

In another embodiment, the invention features a method for generating DFO molecules with increased delivery into a target cell or tissue, such as hepatocytes, endothelial cells, T-cells, primary cells, and neuronal cells, comprising (a) introducing chemical modifications, conjugates, or nucleotides having any of Formula III-IX or any combination thereof into a DFO molecule, and (b) assaying the DFO molecule of step (a) under conditions suitable for isolating DFO molecules having increased delivery into a target cell or tissue. In one embodiment, the invention features DFO duplex constructs that mediate modulation or inhiibtion of gene expression against a target gene, wherein the DFO construct comprises one or more chemical modifications described herein that modulates the binding affinity between the two strands of the DFO construct.

In one embodiment, the binding affinity between the strands of the duplex formed by the DFO of the invention is modulated to increase the activity of the DFO molecule with regard to the ability of the DFO to modulate gene expression. In another embodiment the binding affinity between the two strands of a DFO duplex is decreased. The binding affinity between the strands of the DFO construct can be decreased by introducing one or more chemically modified nucleotides in the DFO sequence that disrupts the duplex stability of the DFO (e.g., lowers the Tm of the duplex). The binding affinity between the strands of the DFO construct can be decreased by introducing one or more nucleotides in the DFO sequence that do not form Watson-Crick base pairs. The binding affinity between the strands of the DFO construct can be decreased by introducing one or more wobble base pairs in the DFO sequence. The binding affinity between the strands of the DFO construct can be decreased by modifying the nucleobase composition of the DFO, such as by altering the G-C content of the DFO sequence (e.g., decreasing the number of G-C base pairs in the DFO sequence). These modifications and alterations in sequence can be introduced selectively at pre-determined positions of the DFO sequence to increase DFO mediated modulation of gene expression. For example, such modifications and sequence alterations can be introduced to disrupt DFO duplex stability between the 5′-end of one strand 3′-end of the other strand, the 3′-end of one strand and the 5′-end of the other strand, or alternately the middle of the DFO duplex. In another embodiment, DFO molecules are screened for optimized activity by introducing such modifications and sequence alterations either by rational design based upon observed rules or trends in increasing DFO activity, or randomly via combinatorial selection processes that cover either partial or complete sequence space of the DFO construct.

In another embodiment, the invention features a method for generating a DFO duplex molecule with increased binding affinity between the strands of the DFO molecule comprising (a) introducing nucleotides having any of Formula III-IX or any combination thereof into a DFO molecule, and (b) assaying the DFO molecule of step (a) under conditions suitable for isolating a DFO molecule having increased binding affinity between the strands of the DFO molecule.

In one embodiment, the invention features a DFO construct that modulates the expression of a target RNA, wherein the DFO construct comprises one or more chemical modifications described herein that modulates the binding affinity between the DFO construct and a complementary target RNA sequence within a cell.

In one embodiment, the invention features a DFO construct that modulates the expression of a target DNA, wherein the DFO construct comprises one or more chemical modifications described herein that modulates the binding affinity between the DFO construct and a complementary target DNA sequence within a cell.

In another embodiment, the invention features a method for generating a DFO molecule with increased binding affinity between the DFO molecule and a complementary target RNA sequence comprising (a) introducing nucleotides having any of Formula III-XI or any combination thereof into a DFO molecule, and (b) assaying the DFO molecule of step (a) under conditions suitable for isolating a DFO molecule having increased binding affinity between the DFO molecule and a complementary target RNA sequence.

In another embodiment, the invention features a method for generating a DFO molecule with increased binding affinity between the DFO molecule and a complementary target DNA sequence comprising (a) introducing nucleotides having any of Formula III-IX or any combination thereof into a DFO molecule, and (b) assaying the DFO molecule of step (a) under conditions suitable for isolating a DFO molecule having increased binding affinity between the DFO molecule and a complementary target DNA sequence.

In one embodiment, the invention features a DFO construct that modulates the expression of a target gene in a cell or reconstituted system, wherein the DFO construct comprises one or more chemical modifications described herein that modulates the cellular uptake of the DFO construct.

In another embodiment, the invention features a method for generating a DFO molecule against a target gene with improved cellular uptake comprising (a) introducing nucleotides having any of Formula III-IX or any combination thereof into a DFO molecule, and (b) assaying the DFO molecule of step (a) under conditions suitable for isolating a DFO molecule having improved cellular uptake.

In one embodiment, the invention features a DFO construct that modulates the expression of a target gene, wherein the DFO construct comprises one or more chemical modifications described herein that increases the bioavailability of the DFO construct, for example, by attaching polymeric conjugates such as polyethyleneglycol or equivalent conjugates that improve the pharmacokinetics of the DFO construct, or by attaching conjugates that target specific tissue types or cell types in vivo. Non-limiting examples of such conjugates are described in Vargeese et al., U.S. Ser. No. 10/201,394 incorporated by reference herein.

In one embodiment, the invention features a method for generating a DFO molecule of the invention with improved bioavailability comprising (a) introducing a conjugate into the structure of a DFO molecule, and (b) assaying the DFO molecule of step (a) under conditions suitable for isolating DFO molecules having improved bioavailability. Such conjugates can include ligands for cellular receptors, such as peptides derived from naturally occurring protein ligands; protein localization sequences, including cellular ZIP code sequences; antibodies; nucleic acid aptamers; vitamins and other co-factors, such as folate and N-acetylgalactosamine; polymers, such as polyethyleneglycol (PEG); phospholipids; cholesterol; polyamines, such as spermine or spermidine; and others.

In one embodiment, the invention features a method for screening DFO molecules against a target nucleic acid sequence comprising, (a) generating a plurality of unmodified DFO molecules, (b) assaying the DFO molecules of step (a) under conditions suitable for isolating DFO molecules that are active in modulating expression of the target nucleic acid sequence, (c) optionally introducing chemical modifications (e.g. chemical modifications as described herein or as otherwise known in the art) into the active DFO molecules of (b), and (d) optionally re-screening the chemically modified DFO molecules of (c) under conditions suitable for isolating chemically modified DFO molecules that are active in modulating expression of the target nucleic acid sequence, for example in a biological system.

In one embodiment, the invention features a method for screening DFO molecules against a target nucleic acid sequence comprising (a) generating a plurality of chemically modified DFO molecules (e.g. DFO molecules as described herein or as otherwise known in the art), and (b) assaying the DFO molecules of step (a) under conditions suitable for isolating chemically modified DFO molecules that are active in modulating expression of the target nucleic acid sequence.

In another embodiment, the invention features a method for generating DFO molecules of the invention with improved bioavailability comprising (a) introducing an excipient formulation to a DFO molecule, and (b) assaying the DFO molecule of step (a) under conditions suitable for isolating DFO molecules having improved bioavailability. Such excipients include polymers such as cyclodextrins, lipids, cationic lipids, polyamines, phospholipids, nanoparticles, receptors, ligands, and others.

In another embodiment, the invention features a method for generating a DFO molecule of the invention with improved bioavailability comprising (a) introducing an excipient formulation to a DFO molecule, and (b) assaying the DFO molecule of step (a) under conditions suitable for isolating DFO molecules having improved bioavailability. Such excipients include polymers such as cyclodextrins, lipids, cationic lipids, polyamines, phospholipids, and others.

In another embodiment, the invention features a method for generating a DFO molecule of the invention with improved bioavailability comprising (a) introducing nucleotides having any of Formulae III-IX, a conjugate, or any combination thereof into a DFO molecule, and (b) assaying the DFO molecule of step (a) under conditions suitable for isolating DFO molecules having improved bioavailability.

In another embodiment, polyethylene glycol (PEG) can be covalently attached to DFO compounds of the present invention. The attached PEG can be any molecular weight, preferably from about 2,000 to about 50,000 daltons (Da).

The present invention can be used alone or as a component of a kit having at least one of the reagents necessary to carry out the in vitro or in vivo introduction of RNA to test samples and/or subjects. For example, preferred components of the kit include a DFO molecule of the invention and a vehicle that promotes introduction of the DFO into cells of interest as described herein (e.g., using lipids and other methods of transfection known in the art, see for example Beigelman et al, U.S. Pat. No. 6,395,713). The kit can be used, for example, for target validation, such as in determining gene function and/or activity, in drug optimization, and in drug discovery (see for example Usman et al., U.S. Ser. No. 60/402,996). Such a kit can also include instructions to allow a user of the kit to practice the invention.

The term “duplex forming oligonucleotide” or “DFO” as used herein refers to any nucleic acid molecule that can form a duplex or a double stranded oligonucleotide in which each strand of the duplex has the same nucleotide sequence.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or down regulating gene expression or viral replication, for example by mediating RNA interference “RNAi” or gene silencing in a sequence-specific manner; see for example Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zemicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). Non limiting examples of siNA molecules of the invention are shown in Table I herein and in Beigelman et al. WO 03/070918. For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic intercations, and/or stacking interactions. In certain embodiments, the siNA molecules of the invention comprise nucleotide sequence that is complementary to nucleotide sequence of a target gene. In another embodiment, the siNA molecule of the invention interacts with nucleotide sequence of a target gene in a manner that causes inhibition of expression of the target gene. As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. Applicant describes in certain embodiments short interfering nucleic acids that do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.” As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (mRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siNA molecules of the invention can be used to epigenetically silence genes at both the post-transcriptional level or the pre-transcriptional level. In a non-limiting example, epigenetic regulation of gene expression by siNA molecules of the invention can result from siNA mediated modification of chromatin structure to alter gene expression (see, for example, Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

By “modulate” is meant that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

By “inhibit”, “down-regulate”, or “reduce”, it is meant that the expression of the gene, or level of RNA molecules or equivalent RNA molecules encoding one or more proteins or protein subunits, or activity of one or more proteins or protein subunits, is reduced below that observed in the absence of the nucleic acid molecules (e.g., DFO) of the invention. In one embodiment, inhibition, down-regulation or reduction with an DFO molecule is below that level observed in the presence of an inactive or attenuated molecule. In another embodiment, inhibition, down-regulation, or reduction with DFO molecules is below that level observed in the presence of, for example, an DFO molecule with scrambled sequence or with mismatches. In another embodiment, inhibition, down-regulation, or reduction of gene expression with a nucleic acid molecule of the instant invention is greater in the presence of the nucleic acid molecule than in its absence.

By “palindrome” or “repeat” nucleic acid sequence is meant, a nucleic acid sequence whose 5′-to-3′ sequence is identical when present in a duplex. For example, a palindrome sequence of the invention in a duplex can comprise sequence having the same sequence when one strand of the duplex is read in the 5′-to-3′ direction (left to right) and the other strand is read 3′- to-5′ direction (right to left). In another example, a repeat sequence of the invention can comprise a sequence having repeated nucleotides so arranged as to provide self complementarity (e.g. 5′-AUAU . . . -3′; 5′-AAUU . . . -3′; 5′-UAUA . . . -3′; 5′-UUAA . . . -3′; 5′-CGCG . . . -3′; 5′-CCGG . . . -3′,5′-GGCC . . . -3′; 5′-CCGG . . . -3′; or any expanded repeat thereof etc., see for example FIG. 4). The palindrome or repeat sequence can comprise about 2 to about 24 nucleotides in even numbers, (e.g., 2, 4, 6, 8, 10, 12, 14, 16, 18, 20, 22, or 24 nucleotides). All that is required of the palindrome or repeat sequence is that it comprises nucleic acid sequence whose 5′-to-3′ sequence is identical when present in a duplex, either alone or as part of a longer nucleic acid sequence. The palindrome or repeat sequence of the invention can comprise chemical modificaitons as described herein that can form, for example, Watson Crick or non-Watson Crick base pairs.

By “gene”, or “target gene”, is meant, a nucleic acid that encodes an RNA, for example, nucleic acid sequences including, but not limited to, structural genes encoding a polypeptide. A gene or target gene can also encode a functional RNA (FRNA) or non-coding RNA (ncRNA), such as small temporal RNA (stRNA), micro RNA (mRNA), small nuclear RNA (snRNA), short interfering RNA (siRNA), small nucleolar RNA (snRNA), ribosomal RNA (rRNA), transfer RNA (tRNA) and precursor RNAs thereof. Such non-coding RNAs can serve as target nucleic acid molecules for DFO mediated RNA interference in modulating the activity of FRNA or ncRNA involved in functional or regulatory cellular processes. Abberant FRNA or ncRNA activity leading to disease can therefore be modulated by DFO molecules of the invention. DFO molecules targeting fRNA and ncRNA can also be used to manipulate or alter the genotype or phenotype of an organism or cell, by intervening in cellular processes such as genetic imprinting, transcription, translation, or nucleic acid processing (e.g., transamination, methylation etc.). The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus. Non-limiting examples of plants include monocots, dicots, or gymnosperms. Non-limiting examples of animals include vertebrates or invertebrates (see for example Zwick et al., U.S. Pat. No. 6,350,934, incorporated by reference herein). Non-limiting examples of fungi include molds or yeasts. Examples of target genes can be found generally in the art, see for example McSwiggen et al., WO 03/74654 and Zwick et al., U.S. Pat. No. 6,350,934, incorporated by reference herein.

By “highly conserved sequence region” is meant, a nucleotide sequence of one or more regions in a target gene does not vary significantly from one generation to the other or from one biological system to the other.

By “cancer” is meant a group of diseases characterized by uncontrolled growth and/or spread of abnormal cells.

By “target nucleic acid” is meant any nucleic acid sequence whose expression or activity is to be modulated. The target nucleic acid can be DNA or RNA, such as endogenous DNA or RNA, viral DNA or viral RNA, or other RNA encoded by a gene, virus, bacteria, fungus, mammal, or plant.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity or inhibition of gene expression or formation of double stranded oligonucleotides by the DFO molecules. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonuelcotide being based paired to a second nucleic acid sequence having 10 nucleotides represents 50%, 60%, 70%, 80%, 90%, and 100% complementary respectively). “Perfectly complementary” or “perfect complementarity” means that all the contiguous residues of a nucleic acid sequence will hydrogen bond with the same number of contiguous residues in a second nucleic acid sequence.

The DFO molecules of the invention represent a novel therapeutic approach to a broad spectrum of diseases and conditions, including cancer or cancerous disease, infectious disease, ocular disease, cardiovascular disease, neurological disease, prion disease, inflammatory disease, autoimmune disease, pulmonary disease, renal disease, liver disease, mitochondrial disease, endocrine disease, reproduction related diseases and conditions, and any other indications that can respond to the level of an expressed gene product or a foreign nucleic acid, such as viral, fungal or bacterial genome, in a cell or organsim.

In one embodiment of the present invention, the sequence of a DFO molecule of the invention is independently about 17 to about 40 nucleotides in length, in specific embodiments about 17, 18, 19, 20, 21, 22, 23, 24, 25, 26, 27, 28, 29 30, 31, 32, 33, 34, 35, 36, 37, 38, 39, or 40 nucleotides in length. In another embodiment, the DFO duplexes of the invention independently comprise about 17 to about 40 base pairs (e.g., about 17, 18, 19, 20, 21, 22, 23, 24, 25, 26, 27, 28, 29 30, 31, 32, 33, 34, 35, 36, 37, 38, 39, or 40 base pairs). Exemplary DFO molecules of the invention are shown in Table I and/or FIGS. 1-3. Non-limiting examples of target sites containing palindromic sequences for VEGFR1, VEGFR2, VEGF, TGFbetaR1, and HIV targets are shown in Table I as well. DFO molecules can be designed to target these sites and such DFO molecules can include chemical modifications as described herein or as otherwise known in the art.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell). The cell can be of somatic or germ line origin, totipotent or pluripotent, dividing or non-dividing. The cell can also be derived from or can comprise a gamete or embryo, a stem cell, or a fully differentiated cell.

The DFO molecules of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In particular embodiments, the nucleic acid molecules of the invention comprise sequences shown in Table I and/or FIGS. 1-3. Examples of such nucleic acid molecules consist essentially of sequences defined in these tables and figures. Furthermore, the chemically modified constructs described in Table IV can be applied to any DFO sequence of the invention.

In another aspect, the invention provides mammalian cells containing one or more DFO molecules of this invention. The one or more DFO molecules can independently be targeted to the same or different sites.

By “RNA” is meant a molecule comprising at least one ribonucleotide residue. By “ribonucleotide” is meant a nucleotide with a hydroxyl group at the 2′ position of a β-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the DFO or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “subject” is meant an organism, which is a donor or recipient of explanted cells or the cells themselves. “Subject” also refers to an organism to which the nucleic acid molecules of the invention can be administered. A subject can be a mammal or mammalian cells, including a human or human cells.

The term “ligand” refers to any compound or molecule, such as a drug, peptide, hormone, or neurotransmitter, that is capable of interacting with another compound, such as a receptor, either directly or indirectly. The receptor that interacts with a ligand can be present on the surface of a cell or can alternately be an intracellular receptor. Interaction of the ligand with the receptor can result in a biochemical reaction, or can simply be a physical interaction or association.

The term “phosphorothioate” as used herein refers to an internucleotide linkage having Formula I, wherein Z and/or W comprise a sulfur atom. Hence, the term phosphorothioate refers to both phosphorothioate and phosphorodithioate internucleotide linkages.

The term “phosphonoacetate” as used herein refers to an internucleotide linkage having Formula I, wherein Z and/or W comprise an acetyl or protected acetyl group.

The term “thiophosphonoacetate” as used herein refers to an internucleotide linkage having Formula I, wherein Z comprises an acetyl or protected acetyl group and W comprises a sulfur atom or alternately W comprises an acetyl or protected acetyl group and Z comprises a sulfur atom.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

The term “acyclic nucleotide” as used herein refers to any nucleotide having an acyclic ribose sugar, for example where any of the ribose carbons (C1, C2, C3, C4, or C5), are independently or in combination absent from the nucleotide.

The nucleic acid molecules of the instant invention, individually, or in combination or in conjunction with other drugs, can be used to treat diseases or conditions discussed herein (e.g., cancers and othe proliferative conditions, viral infection, inflammatory disease, autoimmunity, pulmonary disease, renal disease, ocular disease, etc.). For example, to treat a particular disease or condition, the DFO molecules can be administered to a subject or can be administered to other appropriate cells evident to those skilled in the art, individually or in combination with one or more drugs under conditions suitable for the treatment.

In one embodiment, the invention features a method for treating or preventing a disease or condition in a subject, wherein the disease or condition is related to angiogenesis or neovascularization, comprising administering to the subject a DFO molecule of the invention under conditions suitable for the treatment or prevention of the disease or condition in the subject, alone or in conjunction with one or more other therapeutic compounds. In another embodiment, the disease or condition resulting from angiogenesis, such as tumor angiogenesis leading to cancer, such as without limitation to breast cancer, lung cancer (including non-small cell lung carcinoma), prostate cancer, colorectal cancer, brain cancer, esophageal cancer, bladder cancer, pancreatic cancer, cervical cancer, head and neck cancer, skin cancers, nasopharyngeal carcinoma, liposarcoma, epithelial carcinoma, renal cell carcinoma, gallbladder adeno carcinoma, parotid adenocarcinoma, ovarian cancer, melanoma, lymphoma, glioma, endometrial sarcoma, and multidrug resistant cancers, diabetic retinopathy, macular degeneration, age related macular degeneration, macular adema, neovascular glaucoma, myopic degeneration, arthritis, psoriasis, endometriosis, female reproduction, verruca vulgaris, angiofibroma of tuberous sclerosis, pot-wine stains, Sturge Weber syndrome, Kippel-Trenaunay-Weber syndrome, Osler-Weber-Rendu syndrome, renal disease such as Autosomal dominant polycystic kidney disease (ADPKD), restenosis, arteriosclerosis, and any other diseases or conditions that are related to gene expression or will respond to RNA interference in a cell or tissue, alone or in combination with other therapies.

In one embodiment, the invention features a method for treating or preventing an ocular disease or condition in a subject, wherein the ocular disease or condition is related to angiogenesis or neovascularization (such as those involving genes in the vascular endothelial growth factor, VEGF pathway or TGF-beta pathway), comprising administering to the subject a DFO molecule of the invention under conditions suitable for the treatment or prevention of the disease or condition in the subject, alone or in conjunction with one or more other therapeutic compounds. In another embodiment, the ocular disease or condition comprises macular degeneration, age related macular degeneration, diabetic retinopathy, macular adema, neovascular glaucoma, myopic degeneration, trachoma, scarring of the eye, cataract, ocular inflammation and/or ocular infections.

In one embodiment, the invention features a method of locally administering (e.g. by injection, such as intraocular, intratumoral, periocular, intracranial, etc., topical administration, catheter or the like) to a tissue or cell (e.g., ocular or retinal, brain, CNS) a double stranded RNA formed by a DFO molecule or a vector expressing DFO molecule, comprising nucleotide sequence that is complementary to nucleotide sequence of target RNA, or a portion thereof, (e.g., target RNA encoding VEGF or a VEGF receptor) comprising contacting said tissue of cell with said double stranded RNA under conditions suitable for said local administration.

In one embodiment, the invention features a method of systemically administering (e.g. by injection, such as subcutaneous, intravenous, topical administration, or the like) to a tissue or cell in a subject, a double stranded RNA formed by a DFO molecule or a vector expressing DFO molecule comprising nucleotide sequence that is complementary to nucleotide sequence of target RNA, or a portion thereof, (e.g., target RNA encoding VEGF or a VEGF receptor) comprising contacting said subject with said double stranded RNA under conditions suitable for said systemic administration.

In one embodiment, the invention features a method for treating or preventing tumor angiogenesis in a subject comprising administering to the subject a DFO molecule of the invention under conditions suitable for the treatment or prevention of tumor angiogenesis in the subject, alone or in conjunction with one or more other therapeutic compounds.

In one embodiment, the invention features a method for treating or preventing viral infection or replication in a subject comprising administering to the subject a DFO molecule of the invention under conditions suitable for the treatment or prevention of viral infection or replication in the subject, alone or in conjunction with one or more other therapeutic compounds.

In one embodiment, the invention features a method for treating or preventing autoimmune disease in a subject comprising administering to the subject a DFO molecule of the invention under conditions suitable for the treatment or prevention of autoimmune disease in the subject, alone or in conjunction with one or more other therapeutic compounds.

In one embodiment, the invention features a method for treating or preventing neurologic disease (e.g., Alzheimer's disease, Huntington disease, Parkinson disease, ALS, multiple sclerosis, epilepsy, etc.) in a subject comprising administering to the subject a DFO molecule of the invention under conditions suitable for the treatment or prevention of neurologic disease in the subject, alone or in conjunction with one or more other therapeutic compounds.

In one embodiment, the invention features a method for treating or preventing inflammation in a subject comprising administering to the subject a DFO molecule of the invention under conditions suitable for the treatment or prevention of inflammation in the subject, alone or in conjunction with one or more other therapeutic compounds.

In a further embodiment, the DFO molecules can be used in combination with other known treatments to treat conditions or diseases discussed above. For example, the described molecules could be used in combination with one or more known therapeutic agents to treat a disease or condition. Non-limiting examples of other therapeutic agents that can be readily combined with a DFO molecule of the invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

In another aspect of the invention, DFO molecules that interact with target RNA molecules and down-regulate gene encoding target RNA molecules (for example target RNA molecules referred to by Genbank Accession numbers herein) are expressed from transcription units inserted into DNA or RNA vectors. The recombinant vectors can be DNA plasmids or viral vectors. DFO expressing viral vectors can be constructed based on, but not limited to, adeno-associated virus, retrovirus, adenovirus, or alphavirus. The recombinant vectors capable of expressing the siNA molecules can be delivered as described herein, and persist in target cells. Alternatively, viral vectors can be used that provide for transient expression of DFO molecules. Such vectors can be repeatedly administered as necessary. Once expressed, the DFO molecules interact with target nucleic acids and down-regulate gene function or expression. Delivery of DFO expressing vectors can be systemic, such as by intravenous or intramuscular administration, by administration to target cells ex-planted from a subject followed by reintroduction into the subject, or by any other means that would allow for introduction into the desired target cell.

In one embodiment, the expression vector comprises a transcription initiation region, a transcription termination region, and a gene encoding at least one DFO. The gene can be operably linked to the initiation region and the termination region, in a manner which allows expression and/or delivery of the DFO. In another embodiment, the expression vector can comprises a transcription initiation region, a transcription termination region, an open reading frame and a gene encoding at least one DFO, wherein the gene is operably linked to the 3′-end of the open reading frame. The gene can be operably linked to the initiation region, the open reading frame and the termination region in a manner which allows expression and/or delivery of the DFO. In another embodiment, the expression vector comprises a transcription initiation region, a transcription termination region, an intron, and a gene encoding at least one DFO. The gene can be operably linked to the initiation region, the intron, and the termination region in a manner which allows expression and/or delivery of the DFO. In yet another embodiment, the expression vector comprises a transcription initiation region, a transcription termination region, an intron, an open reading frame, and a gene encoding at least one DFO, wherein the gene is operably linked to the 3′-end of the open reading frame. The gene can be operably linked to the initiation region, the intron, the open reading frame and the termination region in a manner which allows expression and/or delivery of the DFO.

The expression vector can be derived from, for example, a retrovirus, an adenovirus, an adeno-associated virus, an alphavirus or a bacterial plasmid as well as other known vectors. The expression vector can be operably linked to a RNA polymerase II promoter element or a RNA polymerase III promoter element. The RNA polymerase III promoter can be derived from, for example, a transfer RNA gene, a U6 small nuclear RNA gene, or a TRZ RNA gene. The DFO transcript can comprise a sequence at its 5′-end homologous to the terminal 27 nucleotides encoded by the U6 small nuclear RNA gene. The library of DFO constructs can be a multimer random library. The multimer random library can comprise at least one DFO.

The DFO of the instant invention can be chemically synthesized, expressed from a vector, or enzymatically synthesized.

By “vectors” is meant any nucleic acid- and/or viral-based technique used to produce, express and/or deliver a desired nucleic acid, such as the DFO molecule of the invention.

Other features and advantages of the invention will be apparent from the following description of the preferred embodiments thereof, and from the claims.

### BRIEF DESCRIPTION OF THE DRAWINGS

FIG. 1A shows a non-limiting example of methodology used to design self complementary DFO constructs utilizing palidrome and/or repeat nucleic acid sequences that are identifed in a target nucleic acid sequence. (i) A palindrome or repeat sequence is identified in a nucleic acid target sequence. (ii) A sequence is designed that is complementary to the target nucleic acid sequence and the palindrome sequence. (iii) An inverse repeat sequence of the non-palindrome/repeat portion of the complementary sequence is appended to the 3′-end of the complementary sequence to generate a self complementary DFO molecule comprising sequence complementary to the nucleic acid target. (iv) The DFO molecule can self-assemble to form a double stranded oligonucleotide. FIG. 1B shows a non-limiting representative example of a duplex forming oligonucleotide sequence. FIG. 1C shows a non-limiting example of the self assembly schematic of a representative duplex forming oligonucleotide sequence. FIG. 1D shows a non-limiting example of the self assembly schematic of a representative duplex forming oligonucleotide sequence followed by interaction with a target nucleic acid sequence resulting in modulation of gene expression.

FIG. 2 shows a non-limiting example of the design of self complementary DFO constructs utilizing palidrome and/or repeat nucleic acid sequences that are incorporated into the DFO constructs that have sequence complementary to any target nucleic acid sequence of interest. Incorporation of these palindrome/repeat sequences allow the design of DFO constructs that form duplexes in which each strand is capable of mediating modulation of target gene expression, for example by RNAi. First, the target sequence is identified. A complementary sequence is then generated in which nucleotide or non-nucleotide modifications (shown as X or Y) are introduced into the complementary sequence that generate an artificial palindrome (shown as XYXYXY in the Figure). An inverse repeat of the non-palindrome/repeat complementary sequence is appended to the 3′-end of the complementary sequence to generate a self complmentary DFO comprising sequence complementary to the nucleic acid target. The DFO can self-assemble to form a double stranded oligonucleotide.

FIG. 3 shows non-limiting examples of the design of self complementary DFO constructs utilizing palidrome and/or repeat nucleic acid sequences that are incorporated into the DFO constructs that have sequence complementary to any target nucleic acid sequence of interest as described in FIG. 2. The palidrome/repeat sequence comprises chemically modified nucleotides that are able to interact with a portion of the target nucleic acid sequence (e.g., use of modified base analogs that can form Watson Crick base pairs or non-Watson Crick base pairs such as 2-aminopurine or 2-amino-1,6-dihydropurine nucleotides or universal nucleotides).

FIG. 4 shows non-limiting exmples of palindrome/repeat sequences that can be utilized in designing DFO molecules of the invention, for example, where Z in Formula I(a) or I(b) comprises sequences shown as palindromic restriction sites. Non-limiting examples of target nucleic acid sequences for HBV, HCV, and human VEGFR1 RNA that contain palindrome/repeat sequences (in bold) are shown.

FIG. 5 shows non-limiting examples of non-Watson Crick base pairs that can be utilized in generating artificial palindrome sequences for designing DFO molecules of the invention.

FIG. 6 shows non-limiting examples of inhibition of VEGFR1 RNA expression using DFO molecules of the invention. Duplex DFO constructs prepared from compound numbers 32808, 32809, 32810, 32811, and 32812 were assayed along with siNA molecules having known activity against VEGFR1 RNA (compound numbers 32748/32755, 33282/32289, 31270/31273), matched chemistry inverted controls (compound numbers 32772/32779, 32296/32303, 31276/31279), and a transfection agent control (LF2K). As shown in the Figure, the self complementary DFO sequence 32812 shows potent inhibition of VEGFR1 RNA. Sequences for compound numbers are shown in Table I.

FIG. 7 shows non-limiting examples of inhibition of HBV RNA expression using DFO molecules of the invention as assayed by HBsAg levels. A duplex DFO construct prepared from compound 32221 and a hairpin formed with the same sequence (32221 fold) was assayed along with a siNA construct having known activity against HBV RNA (compound number 31335/31337), a matched chemistry inverted control (compound number 31336/31338), and untreated cells (Untreated). As shown in the Figure, the self complementary DFO sequence 32221 shows significant inhibition of HBV HBsAg as a duplex. Sequences for compound numbers are shown in Table I.

FIG. 8 shows non-limiting examples of different stabilization chemistries (1-10) that can be used, for example, to stabilize the 3′-end of DFO sequences of the invention, including (1) [3-3′]-inverted deoxyribose; (2) deoxyribonucleotide; (3) [5′-3′]-3′-deoxyribonucleotide; (4) [5′-3′]-ribonucleotide; (5) [5′-3′]-3′-O-methyl ribonucleotide; (6) 3′-glyceryl; (7) [3′-5′]-3′-deoxyribonucleotide; (8) [3′-3′]-deoxyribonucleotide; (9) [5′-2′]-deoxyribonucleotide; and (10) [5-3′]-dideoxyribonucleotide. In addition to modified and unmodified backbone chemistries indicated in the figure, these chemistries can be combined with different backbone modifications as described herein, for example, backbone modifications having Formula III. In addition, the 2′-deoxy nucleotide shown 5′ to the terminal modifications shown can be another modified or unmodified nucleotide or non-nucleotide described herein, for example modifications having any of Formulae III-IX or any combination thereof.

FIG. 9 shows non-limiting examples of chemically modified terminal phosphate groups of the invention.

FIG. 10A-C is a diagrammatic representation of a scheme utilized in generating an expression cassette to generate DFO constructs. FIG. 10A: A DNA oligomer is synthesized with a 5′-restriction (R1) site sequence followed by a region having sequence identical to a predetermined target sequence, wherein the sense region comprises, for example, about 19, 20, 21, 22, 23, 24, 25, 26, 27, 28, 29, or 30 nucleotides (N) in length, and which is followed by a 3′-restriction site (R2) which is adjacent to a loop sequence of defined sequence (X). FIG. 10B: The synthetic construct is then extended by DNA polymerase to generate a hairpin structure having self-complementary sequence. FIG. 10C: The construct is processed by restriction enzymes specific to R1 and R2 to generate a double-stranded DNA which is then inserted into an appropriate vector for expression in cells. The transcription cassette is designed such that a U6 promoter region flanks each side of the dsDNA which generates the strands of the DFO. Poly T termination sequences can be added to the constructs to generate U overhangs in the resulting transcript.

### DETAILED DESCRIPTION OF THE INVENTION

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small” refers to nucleic acid motifs no more than 100 nucleotides in length, preferably no more than 80 nucleotides in length, and most preferably no more than 50 nucleotides in length; e.g., individual DFO oligonucleotide sequences) are preferably used for exogenous delivery. The simple structure of these molecules increases the ability of the nucleic acid to invade targeted regions of protein and/or RNA structure. Exemplary molecules of the instant invention are chemically synthesized, and others can similarly be synthesized.

Oligonucleotides (e.g., certain modified oligonucleotides or portions of oligonucleotides lacking ribonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 second coupling step for 2′-deoxy nucleotides or 2′-deoxy-2′-fluoro nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 mmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNA-based oligonucleotides is performed as follows: the polymer-bound trityl-on oligoribonucleotide is transferred to a 4 mL glass screw top vial and suspended in a solution of 40% aqueous methylamine (1 mL) at 65° C. for 10 minutes. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the oligoribonucleotide, are dried to a white powder.

The method of synthesis used for RNA including certain DFO molecules of the invention follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 mmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 lmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 mmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 minutes. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA-3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 minutes. The vial is brought to room temperature TEA·3HF (0.1 mL) is added and the vial is heated at 65° C. for 15 minutes. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 minutes. The cartridge is then washed again with water, salt exchanged with 1 M NaCl and washed with water again. The oligonucleotide is then eluted with 30% acetonitrile.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format.

Alternatively, the nucleic acid molecules of the present invention can be synthesized separately and assembled together to form a duplex or joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

A DFO molecule can also be assembled from two distinct nucleic acid strands or fragments wherein the two fragments comprise the same nucleic acid sequence and are self complementary.

DFO constructs can be purified by gel electrophoresis using general methods or can be purified by high pressure liquid chromatography (HPLC; see Wincott et al., supra, the totality of which is hereby incorporated herein by reference) and re-suspended in water.

In another aspect of the invention, DFO molecules of the invention are expressed from transcription units inserted into DNA or RNA vectors. The recombinant vectors can be DNA plasmids or viral vectors. DFO expressing viral vectors can be constructed based on, but not limited to, adeno-associated virus, retrovirus, adenovirus, or alphavirus. The recombinant vectors capable of expressing the DFO molecules can be delivered as described herein, and persist in target cells. Alternatively, viral vectors can be used that provide for transient expression of DFO molecules.

Alternatively, certain DFO molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Thompson et al., 1995, Nucleic Acids Res., 23, 2259; Good et al., 1997, Gene Therapy, 4, 45). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856).

In another aspect of the invention, DFO molecules of the present invention can be expressed from transcription units (see for example Couture et al., 1996, TIG., 12, 510) inserted into DNA or RNA vectors. The recombinant vectors can be DNA plasmids or viral vectors. DFO expressing viral vectors can be constructed based on, but not limited to, adeno-associated virus, retrovirus, adenovirus, or alphavirus. In another embodiment, pol III based constructs are used to express nucleic acid molecules of the invention (see for example Noonberg et al., 5,624,803, Thompson, U.S. Pat. Nos. 5,902,880 and 6,146,886). The recombinant vectors capable of expressing the DFO molecules can be delivered as described above, and persist in target cells. Alternatively, viral vectors can be used that provide for transient expression of nucleic acid molecules. Such vectors can be repeatedly administered as necessary. Once expressed, the DFO molecule interacts with the target mRNA and generates an RNAi response. Delivery of DFO molecule expressing vectors can be systemic, such as by intravenous or intra-muscular administration, by administration to target cells ex-planted from a subject followed by reintroduction into the subject, or by any other means that would allow for introduction into the desired target cell (for a review see Couture et al., 1996, TIG., 12, 510).

In one aspect the invention features an expression vector comprising a nucleic acid sequence encoding at least one DFO molecule of the instant invention. The expression vector can encode the self complementary DFO sequence that can self assemble upon expression from the vector into a duplex oligonucleotide. The nucleic acid sequences encoding the DFO molecules of the instant invention can be operably linked in a manner that allows expression of the DFO molecule (see for example Noonberg et al., 5,624,803; Thompson, U.S. Pat. Nos. 5,902,880 and 6,146,886; Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, 8, 681-686).

In another aspect, the invention features an expression vector comprising: a) a transcription initiation region (e.g., eukaryotic pol I, II or III initiation region); b) a transcription termination region (e.g., eukaryotic pol I, II or III termination region); and c) a nucleic acid sequence encoding at least one of the DFO molecules of the instant invention, wherein said sequence is operably linked to said initiation region and said termination region, in a manner that allows expression and/or delivery of the DFO molecule. The vector can optionally include an open reading frame (ORF) for a protein operably linked on the 5′ side or the 3′-side of the sequence encoding the DFO of the invention; and/or an intron (intervening sequences).

Transcription of the DFO molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J, 11, 4411-8; Lisziewicz et al., 1993, Proc. Natl. Acad. Sci. U S. A, 90, 80004; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as DFO in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736. The above DFO transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect, the invention features an expression vector comprising a nucleic acid sequence encoding at least one of the DFO molecules of the invention, in a manner that allows expression of that DFO molecule. The expression vector comprises in one embodiment; a) a transcription initiation region; b) a transcription termination region; and c) a nucleic acid sequence encoding at least one strand of the DFO molecule, wherein the sequence is operably linked to the initiation region and the termination region in a manner that allows expression and/or delivery of the DFO molecule.

In another embodiment, the expression vector comprises: a) a transcription initiation region; b) a transcription termination region; c) an open reading frame; and d) a nucleic acid sequence encoding at least one strand of a DFO molecule, wherein the sequence is operably linked to the 3′-end of the open reading frame and wherein the sequence is operably linked to the initiation region, the open reading frame and the termination region in a manner that allows expression and/or delivery of the DFO molecule. In yet another embodiment, the expression vector comprises: a) a transcription initiation region; b) a transcription termination region; c) an intron; and d) a nucleic acid sequence encoding at least one DFO molecule, wherein the sequence is operably linked to the initiation region, the intron and the termination region in a manner which allows expression and/or delivery of the nucleic acid molecule.

In another embodiment, the expression vector comprises: a) a transcription initiation region; b) a transcription termination region; c) an intron; d) an open reading frame; and e) a nucleic acid sequence encoding at least one strand of a DFO molecule, wherein the sequence is operably linked to the 3′-end of the open reading frame and wherein the sequence is operably linked to the initiation region, the intron, the open reading frame and the termination region in a manner which allows expression and/or delivery of the DFO molecule.

Optimizing Activity of the Nucleic Acid Molecule of the Invention.

Chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; Burgin et al., supra, and Beigelman et al., WO 03/70918, all of which are incorporated by reference herein). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al, 1996, Biochemistry, 35, 14090). Sugar modification of nucleic acid molecules have been extensively described in the art (see Eckstein et al., International Publication PCT No. WO 92/07065; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Beigelman et al., WO 03/70918; Usman et al., U.S. Pat. No. 5,627,053; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into nucleic acid molecules without modulating catalysis, and are incorporated by reference herein. In view of such teachings, similar modifications can be used as described herein to modify the DFO nucleic acid molecules of the instant invention so long as the ability of DFO to promote RNAI is cells is not significantly inhibited.

While chemical modification of oligonucleotide internucleotide linkages with phosphorothioate, phosphorodithioate, and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

DFO molecules having chemical modifications that maintain or enhance activity are provided. Such a nucleic acid is also generally more resistant to nucleases than an unmodified nucleic acid. Accordingly, the in vitro and/or in vivo activity should not be significantly lowered. In cases in which modulation is the goal, therapeutic nucleic acid molecules delivered exogenously should optimally be stable within cells until translation of the target RNA has been modulated long enough to reduce the levels of the undesirable protein. This period of time varies between hours to days depending upon the disease state. Improvements in the chemical synthesis of RNA and DNA (Wincott et al., 1995, Nucleic Acids Res. 23, 2677; Caruthers et al., 1992, Methods in Enzymology 211,3-19 (incorporated by reference herein)) have expanded the ability to modify nucleic acid molecules by introducing nucleotide modifications to enhance their nuclease stability, as described above.

In one embodiment, nucleic acid molecules of the invention include one or more (e.g., about 1, 2, 3, 4, 5, 6, 7, 8, 9, 10, or more) G-clamp nucleotides. A G-clamp nucleotide is a modified cytosine analog wherein the modifications confer the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets, complementary sequences, or template strands. In another embodiment, nucleic acid molecules of the invention include one or more (e.g., about 1, 2, 3, 4, 5, 6, 7, 8, 9, 10, or more) LNA “locked nucleic acid” nucleotides such as a 2′,4′-C methylene bicyclo nucleotide (see for example Wengel et al., International PCT Publication No. WO 00/66604 and WO 99/14226).

In another embodiment, the invention features conjugates and/or complexes of DFO molecules of the invention. Such conjugates and/or complexes can be used to facilitate delivery of DFO molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention (see for example WO WO 02/094185 and U.S. Ser. No. 10/427,160 both incorporated by reference herein in their entirety including the drawings). The present invention encompasses the design and synthesis of novel conjugates and complexes for the delivery of molecules, including, but not limited to, small molecules, lipids, cholesterol, phospholipids, nucleosides, nucleotides, nucleic acids, antibodies, toxins, negatively charged polymers and other polymers, for example, proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The present invention features compositions and conjugates to facilitate delivery of molecules into a biological system such as cells. The conjugates provided by the instant invention can impart therapeutic activity by transferring therapeutic compounds across cellular membranes. The present invention encompasses the design and synthesis of novel agents for the delivery of molecules, including but not limited to DFO molecules. In general, the transporters described are designed to be used either individually or as part of a multi-component system. The compounds of the invention generally shown in Formulae herein are expected to improve delivery of molecules into a number of cell types originating from different tissues, in the presence or absence of serum.

In another embodiment, the compounds of the invention are provided as a surface component of a lipid aggregate, such as a liposome encapsulated with the predetermined molecule to be delivered. Liposomes, which can be unilamellar or multilamellar, can introduce encapsulated material into a cell by different mechanisms. For example, the liposome can directly introduce its encapsulated material into the cell cytoplasm by fusing with the cell membrane. Alternatively, the liposome can be compartmentalized into an acidic vacuole (i.e., an endosome) and its contents released from the liposome and out of the acidic vacuole into the cellular cytoplasm.

In one embodiment the invention features a lipid aggregate formulation of the compounds described herein, including phosphatidylcholine (of varying chain length; e.g., egg yolk phosphatidylcholine), cholesterol, a cationic lipid, and 1,2-distearoyl-sn-glycero-3-phosphoethanolamine-polythyleneglycol-2000 (DSPE-PEG2000). The cationic lipid component of this lipid aggregate can be any cationic lipid known in the art such as dioleoyl 1,2,-diacyl-3-trimethylammonium-propane (DOTAP). In another embodiment this cationic lipid aggregate comprises a covalently bound compound described in any of the Formulae herein.

In another embodiment, polyethylene glycol (PEG) is covalently attached to the compounds of the present invention. The attached PEG can be any molecular weight but is preferably between 2000-50,000 daltons.

The compounds and methods of the present invention are useful for introducing nucleotides, nucleosides, nucleic acid molecules, lipids, peptides, proteins, and/or non-nucleosidic small molecules into a cell. For example, the invention can be used for nucleotide, nucleoside, nucleic acid, lipids, peptides, proteins, and/or non-nucleosidic small molecule delivery where the corresponding target site of action exists intracellularly.

In one embodiment, the compounds of the instant invention provide conjugates of molecules that can interact with cellular receptors, such as high affinity folate receptors and ASGPr receptors, and provide a number of features that allow the efficient delivery and subsequent release of conjugated compounds across biological membranes. The compounds utilize chemical linkages between the receptor ligand and the compound to be delivered of length that can interact preferentially with cellular receptors. Furthermore, the chemical linkages between the ligand and the compound to be delivered can be designed as degradable linkages, for example by utilizing a phosphate linkage that is proximal to a nucleophile, such as a hydroxyl group. Deprotonation of the hydroxyl group or an equivalent group, as a result of pH or interaction with a nuclease, can result in nucleophilic attack of the phosphate resulting in a cyclic phosphate intermediate that can be hydrolyzed. This cleavage mechanism is analogous RNA cleavage in the presence of a base or RNA nuclease. Alternately, other degradable linkages can be selected that respond to various factors such as UV irradiation, cellular nucleases, pH, temperature etc. The use of degradable linkages allows the delivered compound to be released in a predetermined system, for example in the cytoplasm of a cell, or in a particular cellular organelle.

The present invention also provides ligand derived phosphoramidites that are readily conjugated to compounds and molecules of interest. Phosphoramidite compounds of the invention permit the direct attachment of conjugates to molecules of interest without the need for using nucleic acid phosphoramidite species as scaffolds. As such, the used of phosphoramidite chemistry can be used directly in coupling the compounds of the invention to a compound of interest, without the need for other condensation reactions, such as condensation of the ligand to an amino group on the nucleic acid, for example at the N6 position of adenosine or a 2′-deoxy-2′-amino function. Additionally, compounds of the invention can be used to introduce non-nucleic acid based conjugated linkages into oligonucleotides that can provide more efficient coupling during oligonucleotide synthesis than the use of nucleic acid-based phosphoramidites. This improved coupling can take into account improved steric considerations of abasic or non-nucleosidic scaffolds bearing pendant alkyl linkages.

Compounds of the invention utilizing triphosphate groups can be utilized in the enzymatic incorporation of conjugate molecules into oligonucleotides. Such enzymatic incorporation is useful when conjugates are used in post-synthetic enzymatic conjugation or selection reactions, (see for example Matulic-Adamic et al., 2000, Bioorg. Med. Chem. Lett., 10, 1299-1302; Lee et al., 2001, NAR., 29, 1565-1573; Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumaretal., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; Kuwabara et al., 2000, Curr. Opin. Chem. Biol., 4, 669).

The term “biodegradable linker” as used herein, refers to a nucleic acid or non-nucleic acid linker molecule that is designed as a biodegradable linker to connect one molecule to another molecule, for example, a biologically active molecule to a DFO molecule of the invention or the strands of a DFO molecule of the invention. The biodegradable linker is designed such that its stability can be modulated for a particular purpose, such as delivery to a particular tissue or cell type. The stability of a nucleic acid-based biodegradable linker molecule can be modulated by using various chemistries, for example combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active DFO molecules either alone or in combination with other molecules contemplated by the instant invention include therapeutically active molecules such as antibodies, cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, DFO, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

The term “alkyl” as used herein refers to a saturated aliphatic hydrocarbon, including straight-chain, branched-chain “isoalkyl”, and cyclic alkyl groups. The term “alkyl” also comprises alkoxy, alkyl-thio, alkyl-thio-alkyl, alkoxyalkyl, alkylamino, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Preferably, the alkyl group has 1 to 12 carbons. More preferably it is a lower alkyl of from about 1 to about 7 carbons, more preferably about 1 to about 4 carbons. The alkyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkenyl groups containing at least one carbon-carbon double bond, including straight-chain, branched-chain, and cyclic groups. Preferably, the alkenyl group has about 2 to about 12 carbons. More preferably it is a lower alkenyl of from about 2 to about 7 carbons, more preferably about 2 to about 4 carbons. The alkenyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkynyl groups containing at least one carbon-carbon triple bond, including straight-chain, branched-chain, and cyclic groups. Preferably, the alkynyl group has about 2 to about 12 carbons. More preferably it is a lower alkynyl of from about 2 to about 7 carbons, more preferably about 2 to about 4 carbons. The alkynyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Alkyl groups or moieties of the invention can also include aryl, alkylaryl, carbocyclic aryl, heterocyclic aryl, amide and ester groups. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from about 1 to about 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

The term “alkoxyalkyl” as used herein refers to an alkyl-O-alkyl ether, for example, methoxyethyl or ethoxymethyl.

The term “alkyl-thio-alkyl” as used herein refers to an alkyl-S-alkyl thioether, for example, methylthiomethyl or methylthioethyl.

The term “amino” as used herein refers to a nitrogen containing group as is known in the art derived from ammonia by the replacement of one or more hydrogen radicals by organic radicals. For example, the terms “aminoacyl” and “aminoalkyl” refer to specific N-substituted organic radicals with acyl and alkyl substituent groups respectively.

The term “alkenyl” as used herein refers to a straight or branched hydrocarbon of a designed number of carbon atoms containing at least one carbon-carbon double bond. Examples of “alkenyl” include vinyl, allyl, and 2-methyl-3-heptene.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkenyl” as used herein refers to a C3-C8 cyclic hydrocarbon containing at least one carbon-carbon double bond. Examples of cycloalkenyl include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “cycloalkyl” as used herein refers to a C3-C8 cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C3-C7 cycloalkyl group attached to the parent molecular moiety through an alkyl group, as defined above. Examples of cycloalkylalkyl groups include cyclopropylmethyl and cyclopentylethyl.

The terms “halogen” or “halo” as used herein refers to indicate fluorine, chlorine, bromine, and iodine.

The term “heterocycloalkyl,” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “C1-C6 hydrocarbyl” as used herein refers to straight, branched, or cyclic alkyl groups having 1-6 carbon atoms, optionally containing one or more carbon-carbon double or triple bonds. Examples of hydrocarbyl groups include, for example, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, vinyl, 2-pentene, cyclopropylmethyl, cyclopropyl, cyclohexylmethyl, cyclohexyl and propargyl. When reference is made herein to C1-C6 hydrocarbyl containing one or two double or triple bonds it is understood that at least two carbons are present in the alkyl for one double or triple bond, and at least four carbons for two double or triple bonds.

The term “phosphorus containing group” as used herein, refers to a chemical group containing a phosphorus atom. The phosphorus atom can be trivalent or pentavalent, and can be substituted with O, H, N, S, C or halogen atoms. Examples of phosphorus containing groups of the instant invention include but are not limited to phosphorus atoms substituted with O, H, N, S, C or halogen atoms, comprising phosphonate, alkylphosphonate, phosphate, diphosphate, triphosphate, pyrophosphate, phosphorothioate, phosphorodithioate, phosphoramidate, phosphoramidite groups, nucleotides and nucleic acid molecules.

The term “degradable linker” as used herein, refers to linker moieties that are capable of cleavage under various conditions. Conditions suitable for cleavage can include but are not limited to pH, UV irradiation, enzymatic activity, temperature, hydrolysis, elimination, and substitution reactions, and thermodynamic properties of the linkage.

The term “photolabile linker” as used herein, refers to linker moieties as are known in the art, that are selectively cleaved under particular UV wavelengths. Compounds of the invention containing photolabile linkers can be used to deliver compounds to a target cell or tissue of interest, and can be subsequently released in the presence of a UV source.

The term “nucleic acid conjugates” as used herein, refers to nucleoside, nucleotide and oligonucleotide conjugates.

The term “lipid” as used herein, refers to any lipophilic compound. Non-limiting examples of lipid compounds include fatty acids and their derivatives, including straight chain, branched chain, saturated and unsaturated fatty acids, carotenoids, terpenes, bile acids, and steroids, including cholesterol and derivatives or analogs thereof.

The term “folate” as used herein, refers to analogs and derivatives of folic acid, for example antifolates, dihydrofloates, tetrahydrofolates, tetrahydorpterins, folinic acid, pteropolyglutamic acid, 1-deza, 3-deaza, 5-deaza, 8-deaza, 10-deaza, 1,5-deaza, 5,10 dideaza, 8,10-dideaza, and 5,8-dideaza folates, antifolates, and pteroic acid derivatives.

The term “compounds with neutral charge” as used herein, refers to compositions which are neutral or uncharged at neutral or physiological pH. Examples of such compounds are cholesterol and other steroids, cholesteryl hemisuccinate (CHEMS), dioleoyl phosphatidyl choline, distearoylphosphotidyl choline (DSPC), fatty acids such as oleic acid, phosphatidic acid and its derivatives, phosphatidyl serine, polyethylene glycol-conjugated phosphatidylamine, phosphatidylcholine, phosphatidylethanolamine and related variants, prenylated compounds including famesol, polyprenols, tocopherol, and their modified forms, diacylsuccinyl glycerols, fusogenic or pore forming peptides, dioleoylphosphotidylethanolamine (DOPE), ceramide and the like.

The term “lipid aggregate” as used herein refers to a lipid-containing composition wherein the lipid is in the form of a liposome, micelle (non-lamellar phase) or other aggregates with one or more lipids.

The term “nitrogen containing group” as used herein refers to any chemical group or moiety comprising a nitrogen or substituted nitrogen. Non-limiting examples of nitrogen containing groups include amines, substituted amines, amides, alkylamines, amino acids such as arginine or lysine, polyamines such as spermine or spermidine, cyclic amines such as pyridines, pyrimidines including uracil, thymine, and cytosine, morpholines, phthalimides, and heterocyclic amines such as purines, including guanine and adenine.

Therapeutic nucleic acid molecules (e.g., DFO molecules) delivered exogenously optimally are stable within cells until reverse transcription of the RNA has been modulated long enough to reduce the levels of the RNA transcript. The nucleic acid molecules are resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

Use of the nucleic acid-based molecules of the invention will lead to better treatment of the disease progression by affording the possibility of combination therapies (e.g., multiple DFO molecules targeted to different genes; nucleic acid molecules coupled with known small molecule modulators; or intermittent treatment with combinations of molecules, including different motifs and/or other chemical or biological molecules). The treatment of subjects with DFO molecules can also include combinations of different types of nucleic acid molecules, such as enzymatic nucleic acid molecules (ribozymes), allozymes, antisense, 2,5-A oligoadenylate, decoys, and aptamers.

In another aspect a DFO molecule of the invention comprises one or more 5′ and/or a 3′-cap structure.

By “cap structure” is meant chemical modifications, which have been incorporated at either terminus of the oligonucleotide (see, for example, Adamic et al., U.S. Pat. No. 5,998,203, and Beigelman et al., WO 03/70918 incorporated by reference herein). These terminal modifications protect the nucleic acid molecule from exonuclease degradation, and can help in delivery and/or localization within a cell. The cap can be present at the 5′-terminus (5′-cap) or at the 3′-terminal (3′-cap) or can be present on both termini. Non-limiting examples of the 5′-cap include, but are not limited to, glyceryl, inverted deoxy abasic residue (moiety); 4′,5′-methylene nucleotide; I-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al., International PCT Publication No. WO 93/15187; Uhlman & Peyman, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents.

In one embodiment, the invention features modified DFO molecules, with phosphate backbone modifications comprising one or more phosphorothioate, phosphonoacetate, and/or thiophosphonoacetate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

By “abasic” is meant sugar moieties lacking a base or having other chemical groups in place of a base at the 1′ position, see for example Adamic et al., U.S. Pat. No. 5,998,203.

By “unmodified nucleoside” is meant one of the bases adenine, cytosine, guanine, thymine, or uracil joined to the 1′ carbon of β-D-ribo-furanose.

By “modified nucleoside” is meant any nucleotide base which contains a modification in the chemical structure of an unmodified nucleotide base, sugar and/or phosphate. Non-limiting examples of modified nucleotides are shown by Formulae I-VII and/or other modifications described herein.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., U.S. Pat. No. 6,248,878, which are both incorporated by reference in their entireties.

Various modifications to nucleic acid DFO structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Administration of Nucleic Acid Molecules

A DFO molecule of the invention can be adapted for use to treat any disease, infection or condition associated with gene expression, and other indications that can respond to the level of gene product in a cell or tissue, alone or in combination with other therapies. For example, a DFO molecule can comprise a delivery vehicle, including liposomes, for administration to a subject, carriers and diluents and their salts, and/or can be present in pharmaceutically acceptable formulations. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and US Patent Application Publication No. U.S. 2002130430), biodegradable nanocapsules, and bioadhesive microspheres, or by proteinaceous vectors (O'Hare and Normand, International PCT Publication No. WO 00/53722). In one embodiment, nucleic acid molecules or the invention are administered via biodegradable implant materials, such as elastic shape memory polymers (see for example Lendelein and Langer, 2002, Science, 296, 1673). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. Many examples in the art describe CNS delivery methods of oligonucleotides by osmotic pump, (see Chun et al., 1998, Neuroscience Letters, 257, 135-138, D'Aldin et al., 1998, Mol. Brain Research, 55, 151-164, Dryden et al., 1998, J. Endocrinol., 157, 169-175, Ghirnikar et al., 1998, Neuroscience Letters, 247, 21-24) or direct infusion (Broaddus et al., 1997, Neurosurg. Focus, 3, article 4). Other routes of delivery include, but are not limited to oral (tablet or pill form) and/or intrathecal delivery (Gold, 1997, Neuroscience, 76, 1153-1158). More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein. The molecules of the instant invention can be used as pharmaceutical agents. Pharmaceutical agents prevent, modulate the occurrence, or treat (alleviate a symptom to some extent, preferably all of the symptoms) of a disease state in a subject.

In addition, the invention features the use of methods to deliver the nucleic acid molecules of the instant invention to hematopoietic cells, including monocytes and lymphocytes. These methods are described in detail by Hartmann et al., 1998, J. Pharmacol. Exp. Ther., 285(2), 920-928; Kronenwett et al., 1998, Blood, 91(3), 852-862; Filion and Phillips, 1997, Biochem. Biophys. Acta., 1329(2), 345-356; Ma and Wei, 1996, Leuk. Res., 20(11/12), 925-930; and Bongartz et al., 1994, Nucleic Acids Research, 22(22), 4681-8. Such methods, as described above, include the use of free oligonucleitide, cationic lipid formulations, liposome formulations including pH sensitive liposomes and immunoliposomes, and bioconjugates including oligonucleotides conjugated to fusogenic peptides, for the transfection of hematopoietic cells with oligonucleotides.

In one embodiment, a compound, molecule, or composition for the treatment of ocular conditions (e.g., macular degeneration, diabetic retinopathy etc.) is administered to a subject intraocularly or by intraocular means. In another embodiment, a compound, molecule, or composition for the treatment of ocular conditions (e.g., macular degeneration, diabetic retinopathy etc.) is administered to a subject periocularly or by periocular means (see for example Ahlheim et al., International PCT publication No. WO 03/24420). In one embodiment, a DFO molecule and/or formulation or composition thereof is administered to a subject intraocularly or by intraocular means. In another embodiment, a DFO molecule and/or formualtion or composition thereof is administered to a subject periocularly or by periocular means. Periocular administration generally provides a less invasive approach to administering DFO molecules and formualtion or composition thereof to a subject (see for example Ahlheim et al., International PCT publication No. WO 03/24420). The use of periocular administraction also minimizes the risk of retinal detachment, allows for more frequent dosing or administraction, provides a clinically relevant route of administraction for macular degeneration and other optic conditions, and also provides the possiblilty of using resevoirs (e.g., implants, pumps or other devices) for drug delivery.

In one embodiment, a DFO molecule of the invention is complexed with membrane disruptive agents such as those described in U.S. Patent Appliaction Publication No. 20010007666, incorporated by reference herein in its entirety including the drawings. In another embodiment, the membrane disruptive agent or agents and the DFO molecule are also complexed with a cationic lipid or helper lipid molecule, such as those lipids described in U.S. Pat. No. 6,235,310, incorporated by reference herein in its entirety including the drawings.

In one embodiment, DFO molecules of the invention are formulated or complexed with polyethylenimine (e.g., linear or branched PEI) and/or polyethylenimine derivatives, including for example grafted PEIs such as galactose PEI, cholesterol PEI, antibody derivatized PEI, and polyethylene glycol PEI (PEG-PEI) derivatives thereof (see for example Ogris et al., 2001, AAPA PharmSci, 3, 1-11; Furgeson et al., 2003, Bioconjugate Chem., 14, 840-847; Kunath et al., 2002, Phramaceutical Research, 19, 810-817; Choi et al., 2001, Bull. Korean Chem. Soc., 22, 46-52; Bettinger et al., 1999, Bioconjugate Chem., 10, 558-561; Peterson et al., 2002, Bioconjugate Chem., 13, 845 854; Erbacher et al., 1999, Journal of Gene Medicine Preprint, 1, 1-18; Godbey et al., 1999., PNAS USA, 96, 5177-5181; Godbey et al., 1999, Journal of Controlled Release, 60, 149-160; Diebold et al., 1999, Journal of Biological Chemistry, 274, 19087-19094; Thomas and Klibanov, 2002, PNAS USA, 99, 14640-14645; and Sagara, U.S. Pat. No. 6,586,524, incorporated by reference herein.

In one embodiment, a DFO molecule of the invention comprises a bioconjugate, for example a nucleic acid conjugate as described in Vargeese et al., U.S. Ser. No. 10/427,160, filed Apr. 30, 2003; U.S. Pat. No. 6,528,631; U.S. Pat. No. 6,335,434; US 6, 235,886; U.S. Pat. No. 6,153,737; U.S. Pat. No. 5,214,136; U.S. Pat. No. 5,138,045, all incorporated by reference herein.

Thus, the invention features a pharmaceutical composition comprising one or more nucleic acid(s) of the invention in an acceptable carrier, such as a stabilizer, buffer, and the like. The polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes that lead to systemic absorption include, without limitation: intravenous, subcutaneous, intraperitoneal, inhalation, oral, intrapulmonary and intramuscular. Each of these administration routes exposes the DFO molecules of the invention to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By “pharmaceutically acceptable formulation” is meant a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, DF et al, 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in a mixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

In one embodiment, the invention comprises compositions suitable for administering nucleic acid molecules of the invention to specific cell types. For example, the asialoglycoprotein receptor (ASGPr) (Wu and Wu, 1987, J. Biol. Chem. 262, 4429-4432) is unique to hepatocytes and binds branched galactose-terminal glycoproteins, such as asialoorosomucoid (ASOR). In another example, the folate receptor is overexpressed in many cancer cells. Binding of such glycoproteins, synthetic glycoconjugates, or folates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987, Glycoconjugate J., 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose, galactosamine, or folate based conjugates to transport exogenous compounds across cell membranes can provide a targeted delivery approach to, for example, the treatment of liver disease, cancers of the liver, or other cancers. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention. Non-limiting examples of such bioconjugates are described in Vargeese et al., U.S. Ser. No. 10/201,394, filed Aug. 13, 2001; and Matulic-Adamic et al., U.S. Ser. No. 10/151,116, filed May 17, 2002. In one embodiment, nucleic acid molecules of the invention are complexed with or covalently attached to nanoparticles, such as Hepatitis B virus S, M, or L evelope proteins (see for example Yamado et al., 2003, Nature Biotechnology, 21, 885). In one embodiment, nucleic acid molecules of the invention are delivered with specificity for human tumor cells, specifically non-apoptotic human tumor cells including for example T-cells, hepatocytes, breast carcinoma cells, ovarian carcinoma cells, melanoma cells, intestinal epithelial cells, prostate cells, testicular cells, non-small cell lung cancers, small cell lung cancers, etc.

Alternatively, certain DFO molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Thompson et al., 1995, Nucleic Acids Res., 23, 2259; Good et al., 1997, Gene Therapy, 4, 45; Noonberg et al., 5,624,803; Thompson, U.S. Pat. Nos. 5,902,880 and 6,146,886; Paul et al., 2002, Nature Biotechnology, 19, 505; Miyagishi and Taira, 2002, Nature Biotechnology, 19, 497; Lee et al., 2002, Nature Biotechnology, 19, 500; for a review see Couture et al., 1996, TIG., 12, 510). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856).

In another aspect of the invention, DFO molecules of the present invention can be expressed from transcription units (see for example Couture et al., 1996, TIG., 12, 510) inserted into DNA or RNA vectors. The recombinant vectors can be DNA plasmids or viral vectors. DFO expressing viral vectors can be constructed based on, but not limited to, adeno-associated virus, retrovirus, adenovirus, or alphavirus. In another embodiment, pol III based constructs are used to express nucleic acid molecules of the invention (see for example Thompson, U.S. Pat. Nos. 5,902,880 and 6,146,886). The recombinant vectors capable of expressing the DFO molecules can be delivered as described above, and persist in target cells. Alternatively, viral vectors can be used that provide for transient expression of nucleic acid molecules. Such vectors can be repeatedly administered as necessary. Once expressed, the DFO molecule interacts with the target mRNA and generates an RNAi response. Delivery of DFO molecule expressing vectors can be systemic, such as by intravenous or intramuscular administration, by administration to target cells ex-planted from a subject followed by reintroduction into the subject, or by any other means that would allow for introduction into the desired target cell (for a review see Couture et al., 1996, TIG., 12, 510).

### EXAMPLES

The following are non-limiting examples showing the selection, isolation, synthesis and activity of nucleic acids of the instant invention.

### Example 1

### Serum Stability of Chemically Modified DFO Constructs

Chemical modifications are introduced into DFO constructs to determine the stability of these constructs compared to native DFO oligonucleotides (or those containing for example two thymidine nucleotide overhangs) in human serum. RNAi stability tests are performed by internally labeling DFO and duplexing by incubating in appropriate buffers to facilitate the formation of duplexes by the DFO. Duplexed DFO constructs are then tested for stability by incubating at a final concentration of 2 μM DFO (strand 2 concentration) in 90% mouse or human serum for time-points of 30 sec, 1 min, 5 min, 30 min, 90 min, 4 hrs 10 min, 16 hrs 24 min, and 49 hrs. Time points are run on a 15% denaturing polyacrylamide gels and analyzed on a phosphoimager.

Internal labeling is performed via kinase reactions with polynucleotide kinase (PNK) and ³²P-γ-ATP, with addition of radiolabeled phosphate at a nucleotide position (e.g. nucleotide 13) of strand 2, counting in from the 3′ side. Ligation of the remaining fragments with T4 RNA ligase results in the full length strand 2. Duplexing of DFO is accomplished for example by adding an appropriate concentration of the DFO oligonucleotide and heating to 95° C. for 5 minutes followed by slow cooling to room temperature. Reactions are performed by adding 100% serum to the DFO duplexes and incubating at 37° C., then removing aliquots at desired time-points.

### Example 2

### Identification of Potential DFO Target Sites in any RNA Sequence

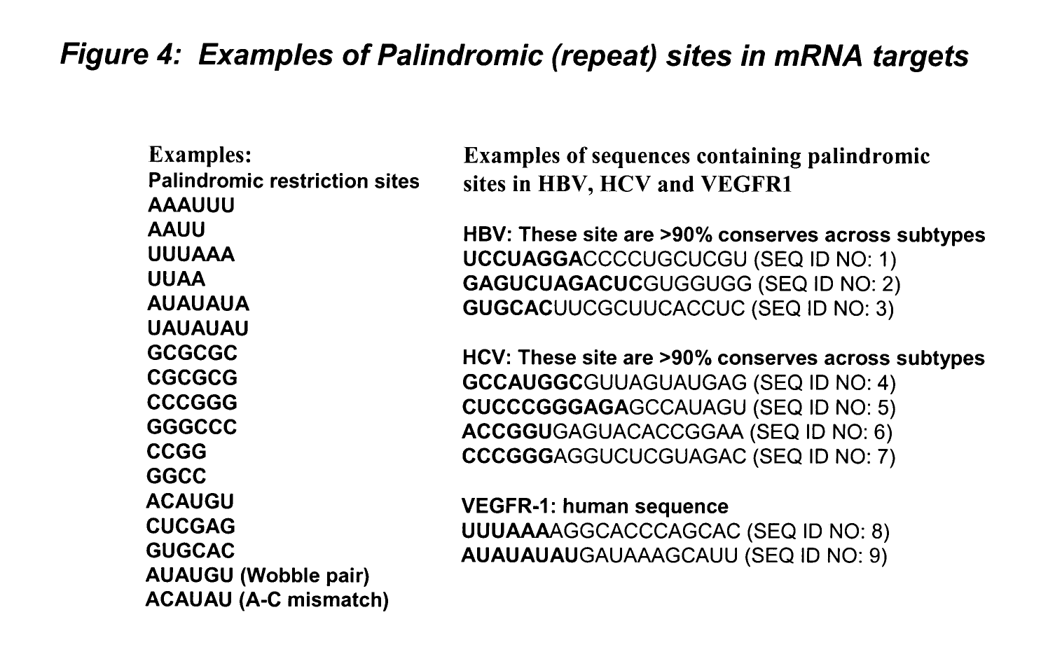

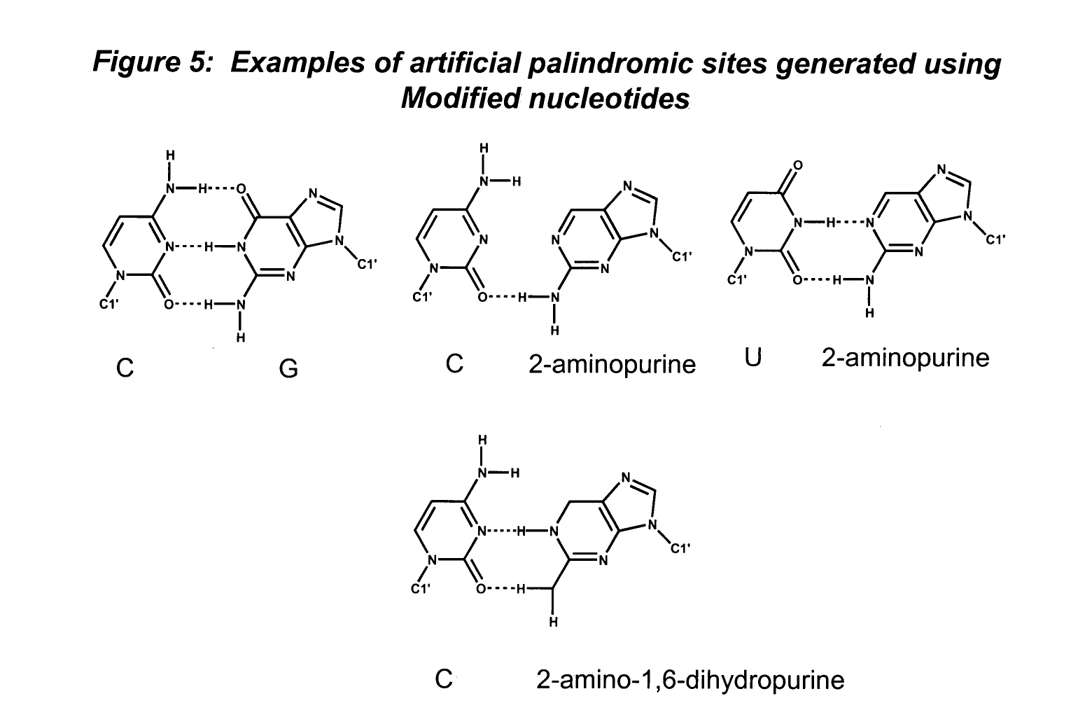

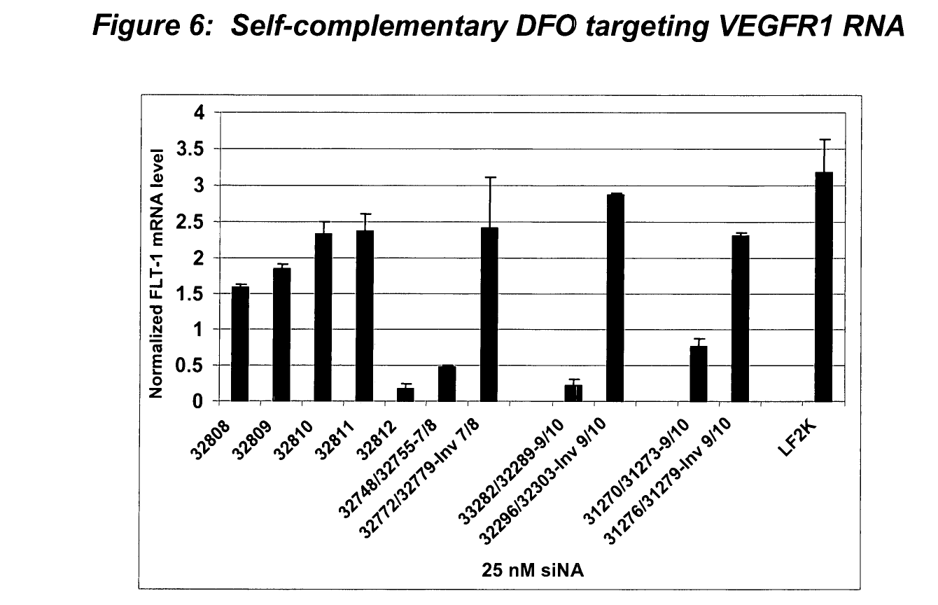

The sequence of an RNA target of interest, such as a viral or human mRNA transcript, is screened for target sites, for example by using a computer folding algorithm. Such target sites can contain palindrome or repeat sequences, for example as shown in FIG. 4. In a non-limiting example, the sequence of a gene or RNA gene transcript derived from a database, such as Genbank, is used to generate DFO targets having complementarity to the target. Such sequences can be obtained from a database, or can be determined experimentally as known in the art. Target sites that are known, for example, those target sites determined to be effective target sites based on studies with other nucleic acid molecules, for example ribozymes or antisense, or those targets known to be associated with a disease or condition such as those sites containing mutations or deletions, can be used to design DFO molecules targeting those sites. Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, or the relative position of the target sequence within the RNA transcript. Based on these determinations, any number of target sites within the RNA transcript can be chosen to screen DFO molecules for efficacy, for example by using in vitro RNA cleavage assays, cell culture, or animal models. In a non-limiting example, anywhere from 1 to 1000 target sites are chosen within the transcript based on the size of the DFO construct to be used. High throughput screening assays can be developed for screening DFO molecules using methods known in the art, such as with multi-well or multi-plate assays or combinatorial/DFO library screening assays to determine efficient reduction in target gene expression.

### Example 3

### Selection of DFO Molecule Target Sites in a RNA

The following non-limiting steps can be used to carry out the selection of DFOs targeting a given gene sequence or transcript.

The target sequence is parsed in silico into a list of all fragments or subsequences containing palindromic or repeat sequences for fragments containing, for example, 2-18 nucleotide repeats contained within the target sequence. This step is typically carried out using a custom Perl script, but commercial sequence analysis programs such as Oligo, MacVector, or the GCG Wisconsin Package can be employed as well.

In some instances, the DFOs correspond to more than one target sequence; such would be the case for example in targeting different transcripts of the same gene, targeting different transcripts of more than one gene, or for targeting both the human gene and an animal homolog. In this case, a subsequence list of a particular length is generated for each of the targets, and then the lists are compared to find matching sequences in each list. The subsequences are then ranked according to the number of target sequences that contain the given subsequence. The goal is to find subsequences that are present in most or all of the target sequences. Alternately, the ranking can identify subsequences that are unique to a target sequence, such as a mutant target sequence. Such an approach would enable the use of DFO to target specifically the mutant sequence and not effect the expression of the normal sequence.

In some instances, the DFO subsequences are absent in one or more sequences while present in the desired target sequence; such would be the case if the DFO targets a gene with a paralogous family member that is to remain untargeted. As in case 2 above, a subsequence list of a particular length is generated for each of the targets, and then the lists are compared to find sequences that are present in the target gene but are absent in the untargeted paralog.

The ranked DFO subsequences can be further analyzed and ranked according to GC content. A preference can be given to sites containing 30-70% GC, with a further preference to sites containing 40-60% GC.

The ranked DFO subsequences can be further analyzed and ranked according to whether they have runs of GGG or CCC in the sequence. GGG (or even more Gs) in either strand can make oligonucleotide synthesis problematic and can potentially interfere with activity, so it is avoided when other appropriately suitable sequences are available. CCC is searched in the target strand because that will place GGG in the DFO strand.

The ranked DFO subsequences can be further analyzed and ranked according to whether they have the dinucleotide UU (uridine dinucleotide) on the 3′-end of the sequence, and/or AA on the 5′-end of the sequence (to yield 3′ UU on the DFO sequence). These sequences allow one to design DFO molecules with terminal TT thymidine dinucleotides.

The DFO molecules are screened in an appropriate in vitro, cell culture or animal model system, such as the systems described herein or otherwise known in the art, to identify the most active DFO molecule or the most preferred target site within the target RNA sequence.

### Example 4

### DFO Design

DFO target sites were chosen by analyzing sequences of the target RNA and optionally prioritizing the target sites on the basis of preferred sequence motifs, such as predicted duplex stability, GC content, folding (structure of any given sequence analyzed to determine DFO accessibility to the target), or by using a library of DFO molecules. DFO molecules were designed that could bind each target and are optionally individually analyzed by computer folding to assess whether the DFO molecule can interact with the target sequence. Varying the length of the DFO molecules can be chosen to optimize activity. Generally, a sufficient number of complementary nucleotide bases are chosen to bind to, or otherwise interact with, the target RNA, but the degree of complementarity can be modulated to accommodate DFO duplexes or varying length or base composition. By using such methodologies, DFO molecules can be designed to target sites within any known RNA sequence, for example those RNA sequences corresponding to the any gene transcript.

Chemically modified DFO constructs are designed to provide nuclease stability for systemic administration in vivo and/or improved pharmacokinetic, localization, and delivery properties while preserving the ability to mediate gene inibition activity. Chemical modifications as described herein are introduced synthetically using synthetic methods described herein and those generally known in the art. The synthetic DFO constructs are then assayed for nuclease stability in serum and/or cellular/tissue extracts (e.g. liver extracts). The synthetic DFO constructs are also tested in parallel for activity using an appropriate assay, such as a luciferase reporter assay as described herein or another suitable assay that can quantity inhibitory activity. Synthetic DFO constructs that possess both nuclease stability and activity can be further modified and re-evaluated in stability and activity assays. The chemical modifications of the stabilized active DFO constructs can then be applied to any DFO sequence targeting any chosen RNA and used, for example, in target screening assays to pick lead DFO compounds for therapeutic development. Alternately, chemically modified DFO constructs can be screened directly for activity in an appropriate assay system (e.g., cell cuture, animal models etc.).

### Example 5

### Chemical Synthesis and Purification of DFO

DFO molecules can be designed to interact with various sites in the RNA message, for example, target sequences within the RNA sequences described herein. The sequence of the DFO molecule(s) is complementary to the target site sequences described above. The DFO molecules can be chemically synthesized using methods described herein. Inactive DFO molecules that are used as control sequences can be synthesized by scrambling the sequence of the DFO molecules such that it is not complementary to the target sequence. Generally, DFO constructs can by synthesized using solid phase oligonucleotide synthesis methods as described herein (see for example Usman et al., U.S. Pat. Nos. 5,804,683; 5,831,071; 5,998,203; 6,117,657; 6,353,098; 6,362,323; 6,437,117; 6,469,158; Scaringe et al., U.S. Pat. Nos. 6,111,086; 6,008,400; 6,111,086 all incorporated by reference herein in their entirety).

In a non-limiting example, RNA oligonucleotides are synthesized in a stepwise fashion using the phosphoramidite chemistry described herein and as is known in the art. Standard phosphoramidite chemistry involves the use of nucleosides comprising any of 5′-O-dimethoxytrityl, 2′-O-tert-butyldimethylsilyl, 3′-O-2-Cyanoethyl N,N-diisopropylphos-phoroamidite groups, and exocyclic amine protecting groups (e.g. N6-benzoyl adenosine, N4 acetyl cytidine, and N2-isobutyryl guanosine). Alternately, 2′-O-Silyl ethers can be used in conjunction with acid-labile 2′-O-orthoester protecting groups in the synthesis of RNA as described by Scaringe supra. Differing 2′ chemistries can require different protecting groups, for example, 2′-deoxy-2′-amino nucleosides can utilize N-phthaloyl protection as described by Usman et al., U.S. Pat. No. 5,631,360, incorporated by reference herein in its entirety).

During solid phase synthesis, each nucleotide is added sequentially (3′- to 5′-direction) to the solid support-bound oligonucleotide. The first nucleoside at the 3′-end of the chain is covalently attached to a solid support (e.g., controlled pore glass or polystyrene) using various linkers. The nucleotide precursor, a ribonucleoside phosphoramidite, and activator are combined resulting in the coupling of the second nucleoside phosphoramidite onto the 5′-end of the first nucleoside. The support is then washed and any unreacted 5′-hydroxyl groups are capped with a capping reagent such as acetic anhydride to yield inactive 5′-acetyl moieties. The trivalent phosphorus linkage is then oxidized to a more stable phosphate linkage. At the end of the nucleotide addition cycle, the 5-O-protecting group is cleaved under suitable conditions (e.g., acidic conditions for trityl-based groups and Fluoride for silyl-based groups). The cycle is repeated for each subsequent nucleotide.

Modification of synthesis conditions can be used to optimize coupling efficiency, for example, by using differing coupling times, differing reagent/phosphoramidite concentrations, differing contact times, differing solid supports and solid support linker chemistries depending on the particular chemical composition of the DFO to be synthesized. Deprotection and purification of the DFO can be performed as is generally described in Usman et al., U.S. Pat. No. 5,831,071, U.S. Pat. No. 6,353,098, U.S. Pat. No. 6,437,117, and Bellon et al., U.S. Pat. No. 6,054,576, U.S. Pat. No. 6,162,909, U.S. Pat. No. 6,303,773, or Scaringe supra, incorporated by reference herein in their entireties. Additionally, deprotection conditions can be modified to provide the best possible yield and purity of DFO constructs. For example, applicant has observed that oligonucleotides comprising 2′-deoxy-2′-fluoro nucleotides can degrade under inappropriate deprotection conditions. Such oligonucleotides are deprotected using aqueous methylamine at about 35° C. for 30 minutes. If the 2′-deoxy-2′-fluoro containing oligonucleotide also comprises ribonucleotides, after deprotection with aqueous methylamine at about 35° C. for 30 minutes, TEA-HF is added and the reaction maintained at about 65° C. for an additional 15 minutes.

### Example 6

### Nucleic Acid Inhibition of Target RNA In Vivo

DFO molecules targeted to the target RNA are designed and synthesized as described above. These nucleic acid molecules can be tested for cleavage activity in vivo, for example, using the following procedure.

Two formats are used to test the efficacy of DFOs targeting a particular gene transcipt. First, the reagents are tested on target expressing cells (e.g., HeLa), to determine the extent of RNA and protein inhibition. DFO reagents are selected against the RNA target. RNA inhibition is measured after delivery of these reagents by a suitable transfection agent to cells. Relative amounts of target RNA are measured versus actin using real-time PCR monitoring of amplification (eg., ABI 7700 Taqman®). A comparison is made to a mixture of oligonucleotide sequences made to unrelated targets or to a randomized DFO control with the same overall length and chemistry, but with randomly substituted nucleotides at each position. Primary and secondary lead reagents are chosen for the target and optimization performed. After an optimal transfection agent concentration is chosen, a RNA time-course of inhibition is performed with the lead DFO molecule. In addition, a cell-plating format can be used to determine RNA inhibition.

Delivery of DFO to Cells

Cells (e.g., HeLa) are seeded, for example, at 1×10⁵ cells per well of a six-well dish in EGM-2 (BioWhittaker) the day before transfection. DFO (final concentration, for example 20 nM) and cationic lipid (e.g., final concentration 2 μg/ml) are complexed in EGM basal media (Biowhittaker) at 37° C. for 30 mins in polystyrene tubes. Following vortexing, the complexed DFO is added to each well and incubated for the times indicated. For initial optimization experiments, cells are seeded, for example, at 1×10³ in 96 well plates and DFO complex added as described. Efficiency of delivery of DFO to cells is determined using a fluorescent DFO complexed with lipid. Cells in 6-well dishes are incubated with DFO for 24 hours, rinsed with PBS and fixed in 2% paraformaldehyde for 15 minutes at room temperature. Uptake of DFO is visualized using a fluorescent microscope.

Tagman and Lightcycler Quantification of mRNA

Total RNA is prepared from cells following DFO delivery, for example, using Qiagen RNA purification kits for 6-well or Rneasy extraction kits for 96-well assays. For Taqman analysis, dual-labeled probes are synthesized with the reporter dye, FAM or JOE, covalently linked at the 5′-end and the quencher dye TAMRA conjugated to the 3′-end. One-step RT-PCR amplifications are performed on, for example, an ABI PRISM 7700 Sequence Detector using 50 μl reactions consisting of 10 μl total RNA, 100 nM forward primer, 900 nM reverse primer, 100 nM probe, 1× TaqMan PCR reaction buffer (PE-Applied Biosystems), 5.5 mM MgCl₂, 300 μM each dATP, dCTP, dGTP, and dTTP, 10U RNase Inhibitor (Promega), 1.25U AmpliTaq Gold (PE-Applied Biosystems) and 10U M-MLV Reverse Transcriptase (Promega). The thermal cycling conditions can consist of 30 min at 48° C., 10 min at 95° C., followed by 40 cycles of 15 sec at 95° C. and 1 min at 60° C. Quantitation of mRNA levels is determined relative to standards generated from serially diluted total cellular RNA (300, 100, 33, 11 ng/rxn) and normalizing to β-actin or GAPDH mRNA in parallel TaqMan reactions. For each gene of interest an upper and lower primer and a fluorescently labeled probe are designed. Real time incorporation of SYBR Green I dye into a specific PCR product can be measured in glass capillary tubes using a lightcyler. A standard curve is generated for each primer pair using control cRNA. Values are represented as relative expression to GAPDH in each sample.

Western Blotting

Nuclear extracts can be prepared using a standard micro preparation technique (see for example Andrews and Faller, 1991, Nucleic Acids Research, 19, 2499). Protein extracts from supernatants are prepared, for example, using TCA precipitation. An equal volume of 20% TCA is added to the cell supernatant, incubated on ice for 1 hour and pelleted by centrifugation for 5 minutes. Pellets are washed in acetone, dried and resuspended in water. Cellular protein extracts are run on a 10% Bis-Tris NuPage (nuclear extracts) or 4-12% Tris-Glycine (supernatant extracts) polyacrylamide gel and transferred onto nitro-cellulose membranes. Non-specific binding can be blocked by incubation, for example, with 5% non-fat milk for 1 hour followed by primary antibody for 16 hour at 4° C. Following washes, the secondary antibody is applied, for example, (1:10,000 dilution) for 1 hour at room temperature and the signal detected with SuperSignal reagent (Pierce).

### Example 7

### Self Complementary DFO Constructs Targeting VEGFR1

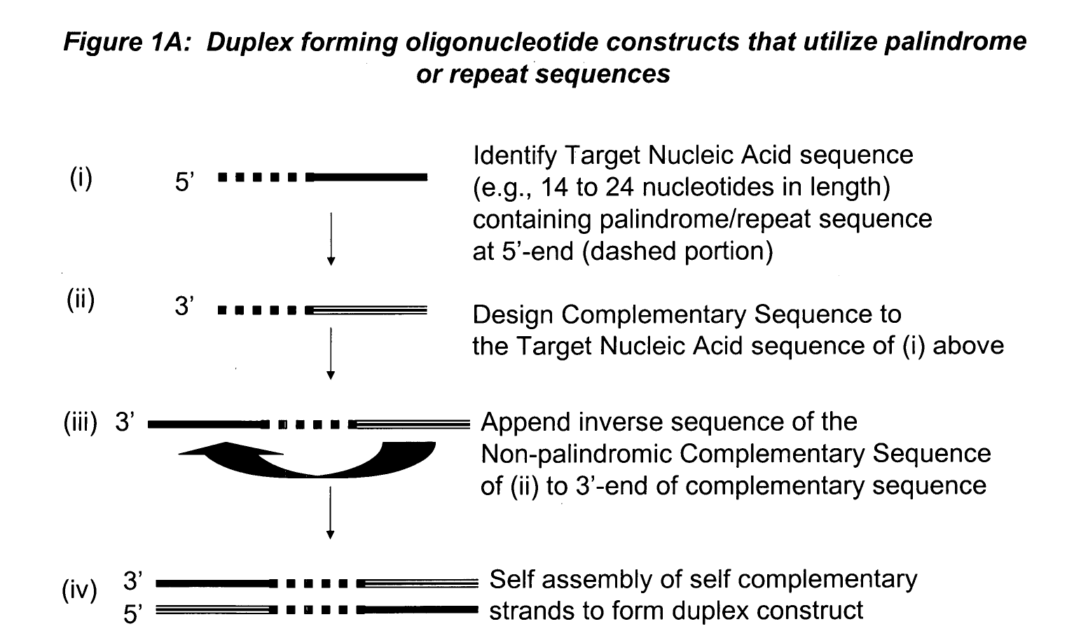

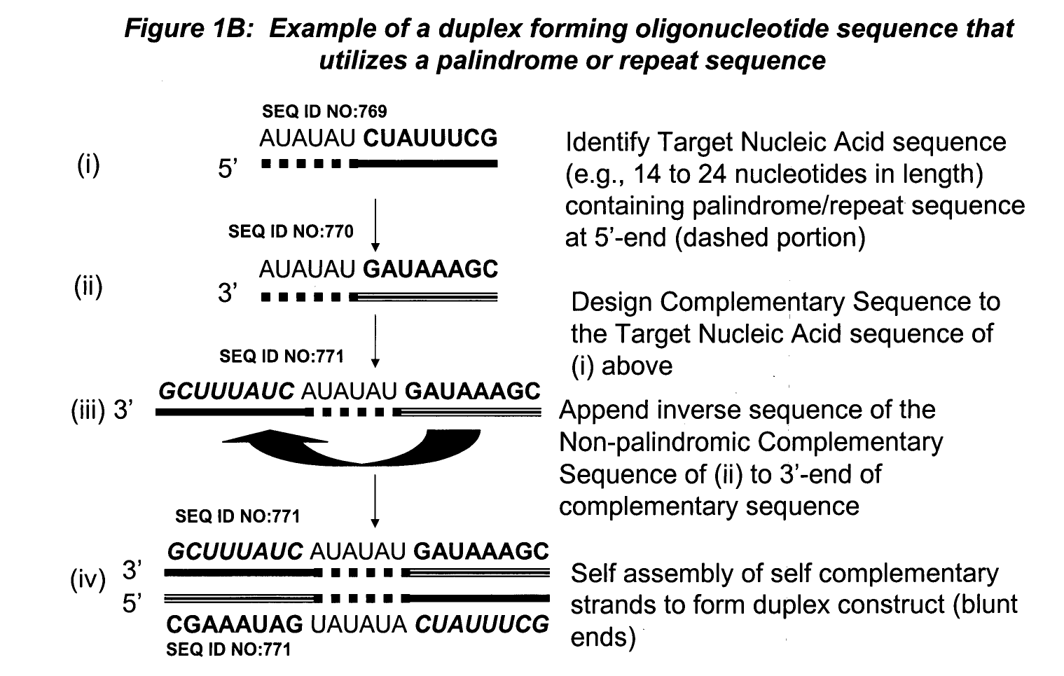

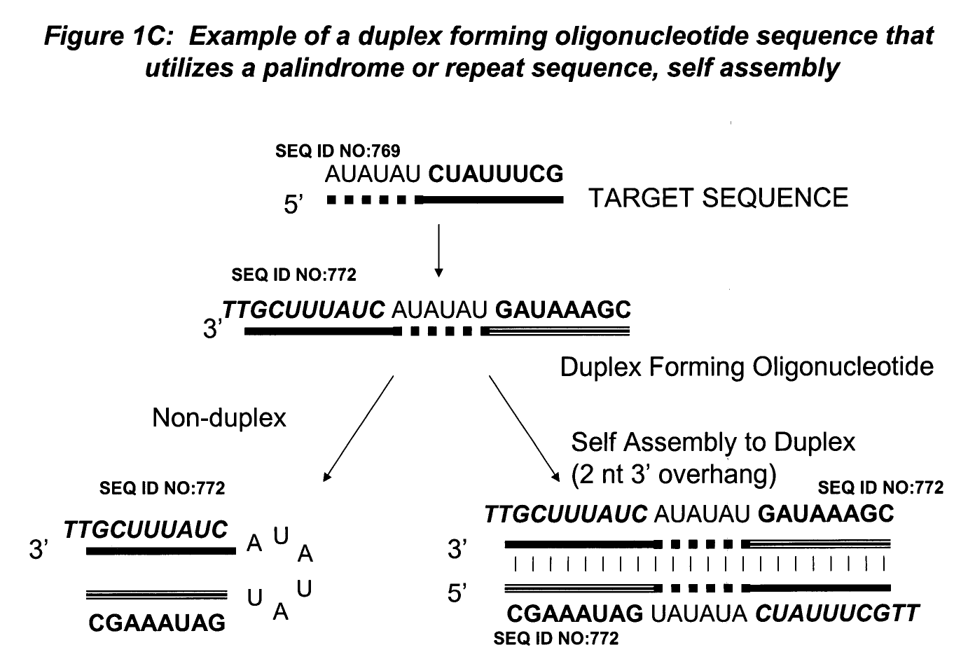

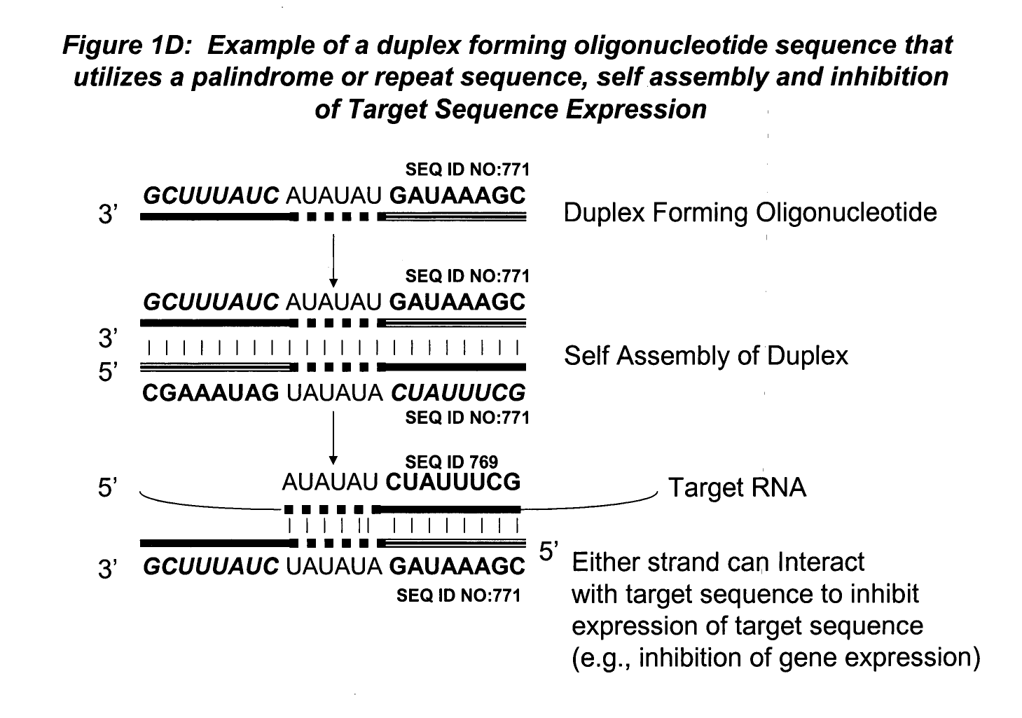

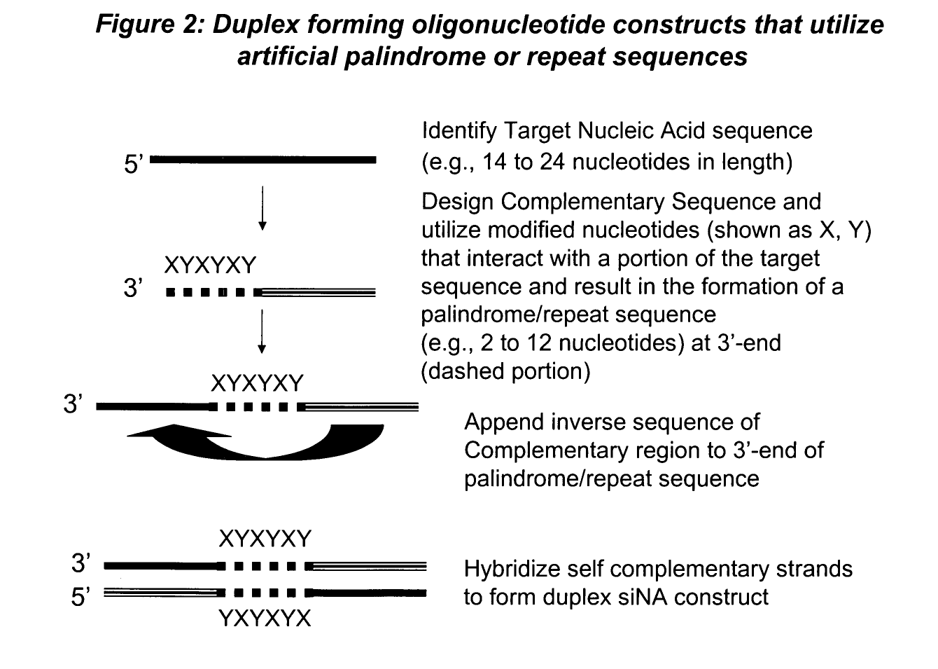

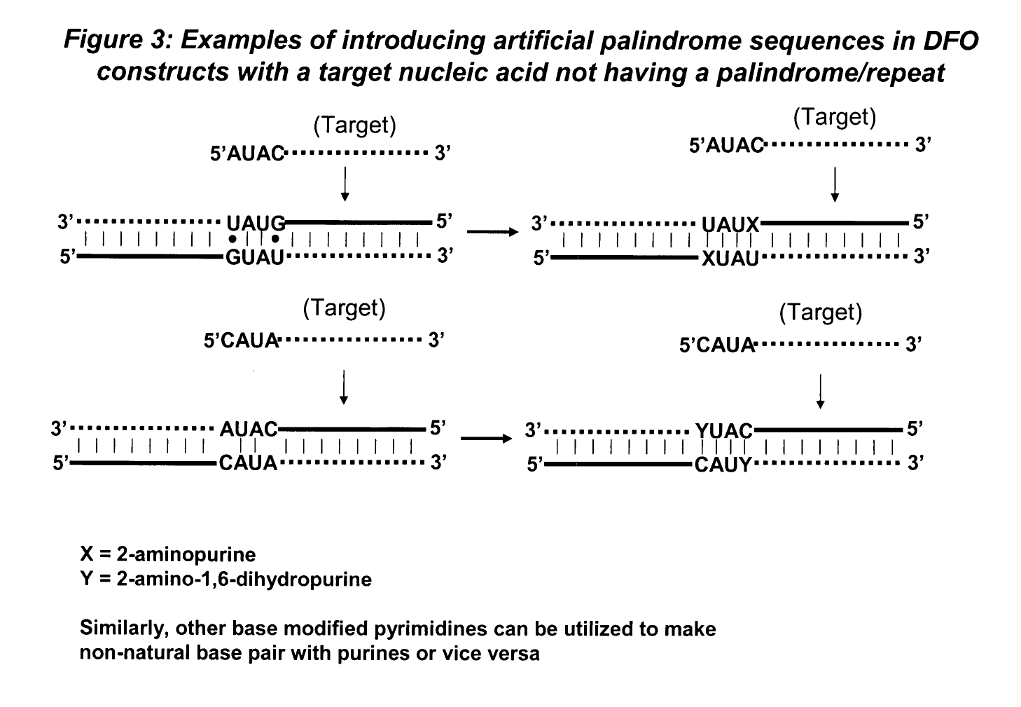

Using the methods described herein, self complementary DFO constructs comprising palindrome or repeat nucleotide sequences were designed against VEGFR1 target RNA. These DFO constructs utilize the identification of palindromic or repeat sequences (for example Z in Formula I(a) and I(b) herein) in a target nucleic acid sequence of interest, generally these palindrome/repeat sequences comprise about 2 to about 12 nucleotids in length are are selected to originate at the 5′-region of the target nucleic acid sequence. A nucleotide sequence that is complementary to target nucleic acid sequence adjacent (3′) to the palindrome/repeat sequence is incorporated at the 5′-end of the palindrome/repeat sequence in the DFO molecule. Lastly, a nucleic sequence that is inverse repeat of the sequence at the 5′ end of the palindrome/repeat sequence is inserted at the 3′ end of the palindrome/repeat sequence such that the full length DFO sequence comprises self complementary sequence. This design of DFO construct allows for the formation of a duplex oligonucleotide in which both strands comprise the same sequence (e.g., see FIG. 1). Generally, the longer the repeat identified in the target nucleic acid sequence, the shorter the resulting DFO sequence will be. For example, if the target sequence is 17 nucleotides in length and there is no repeat found in the sequence, the resulting DFO construct will be, for example, 17+0+17=34 nucleotides in length. The first 17 nucleotides represent sequence complementary to the target nucleic acid sequence, the 0 represents the lack of a palindrome sequence, and the second 17 nucleotides represent inverted repeat sequence of the first 17 nucleotides. If a 2 nucleotide repeat is utilized, the resulting DFO construct will be, for example, 15+2+15=32 nucleotides in length. If a 4 nucleotide repeat is utilized, the resulting DFO construct will be, for example, 13+4+13=30 nucleotides in length. If a 6 nucleotide repeat is utilized, the resulting DFO construct will be, for example, 11+6+11=28 nucleotides in length. If a 8 nucleotide repeat is utilized, the resulting DFO construct will be, for example, 9+8+9=26 nucleotides in length. If a 10 nucleotide repeat is utilized, the resulting DFO construct will be, for example, 7+10+7=24 nucleotides in length. If a 12 nucleotide repeat is utilized, the resulting DFO construct will be, for example, 5+12+5=22 nucleotides in length and so forth. Thus, for each nucleotide reduction in the target site, the DFO length can be shortened by 2 nucleotides. These same principles can be utilized for a target site having different length nucleotide sequences, such as target sites comprising 14 to 24 nucleotides. In addition, various combinations of 5′ and 3′ overhang sequences (e.g., TT) can be introduced to the DFO constructs designed with palindrome/repeat sequences. Furthermore, palindrome/repeat sequences can be added to the 5′-end of a DFO sequence having complementarity to any target nucleic acid sequence of interest, enabling self complementary palindrome/repeat DFO constructs to be designed against any target nucleic acid sequence (see for example FIGS. 2-3).

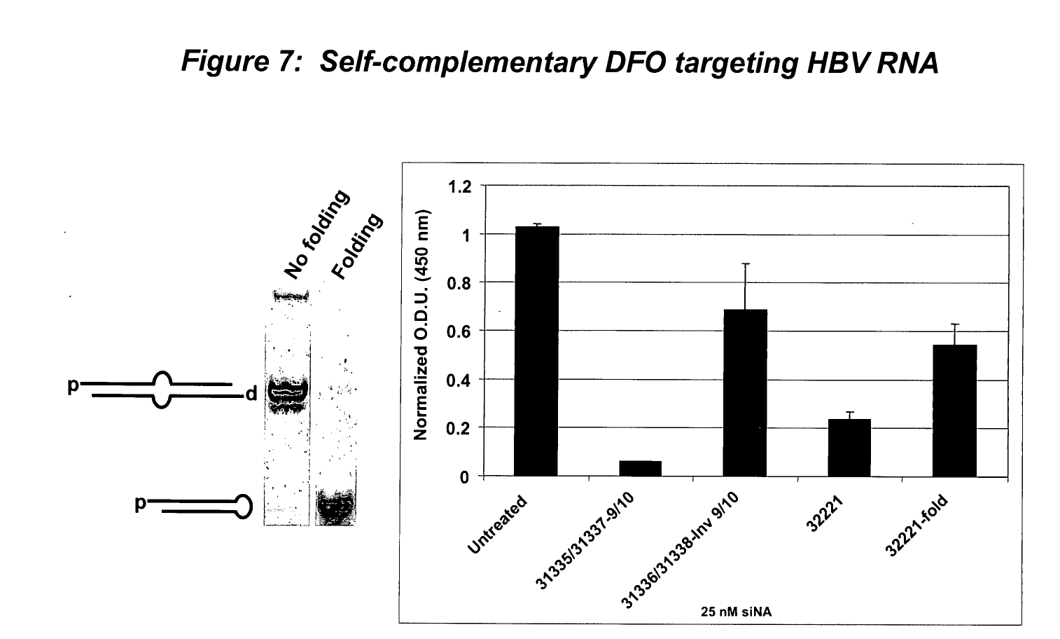

Self complementary DFO palindrome/repeat sequences shown in Table I (compound # 32808, 32809, 32810, 32811, and 32812) were designed against VEGFR1 RNA targets and were screened in cell culture experiments along with chemically modified siNA constructs (compound #s 32748/32755, 33282/32289, 31270/31273) with known activity with matched chemistry inverted controls (compound #s 32772/32779, 32296/32303, 31276/31279) and untreated cells along with a trasfection control (LF2K), see FIG. 7. HAEC cells were transfected with 0.25 μg/well of lipid complexed with 25 nM DFO targeting VEGFR1 site 1229. Cells were incubated at 37° for 24 h in the continued presence of the DFO transfection mixture. At 24 h, RNA was prepared from each well of treated cells. The supernatants with the transfection mixtures were first removed and discarded, then the cells were lysed and RNA prepared from each well. Target gene expression following treatment was evaluated by RT-PCR for the VEGFR1 mRNA and for a control gene (36B4, an RNA polymerase subunit) for normalization. Compound # 32812, a 29 nucleotide self complementary DFO construct targeting VEGFR1 site 1229 displayed potent inhibition of VEGFR1 RNA expression in this system (see for example FIG. 7).

### Example 8

### Self Complementary DFO Constructs Targeting HBV RNA

Self complementary DFO constructs comprising palindrome or repeat nucleotide sequences (see Table I) were designed against HBV target RNA and were screened in HepG2 cells. Transfection of the human hepatocellular carcinoma cell line, Hep G2, with replication-competent HBV DNA results in the expression of HBV proteins and the production of virions. The human hepatocellular carcinoma cell line Hep G2 was grown in Dulbecco's modified Eagle media supplemented with 10% fetal calf serum, 2 mM glutamine, 0.1 mM nonessential amino acids, 1 mM sodium pyruvate, 25 mM Hepes, 100 units penicillin, and 100 μg/ml streptomycin. To generate a replication competent cDNA, prior to transfection the HBV genomic sequences are excised from the bacterial plasmid sequence contained in the psHBV-I vector. Other methods known in the art can be used to generate a replication competent cDNA. This was done with an EcoRI and Hind III restriction digest. Following completion of the digest, a ligation was performed under dilute conditions (20 μg/ml) to favor intermolecular ligation. The total ligation mixture was then concentrated using Qiagen spin columns.

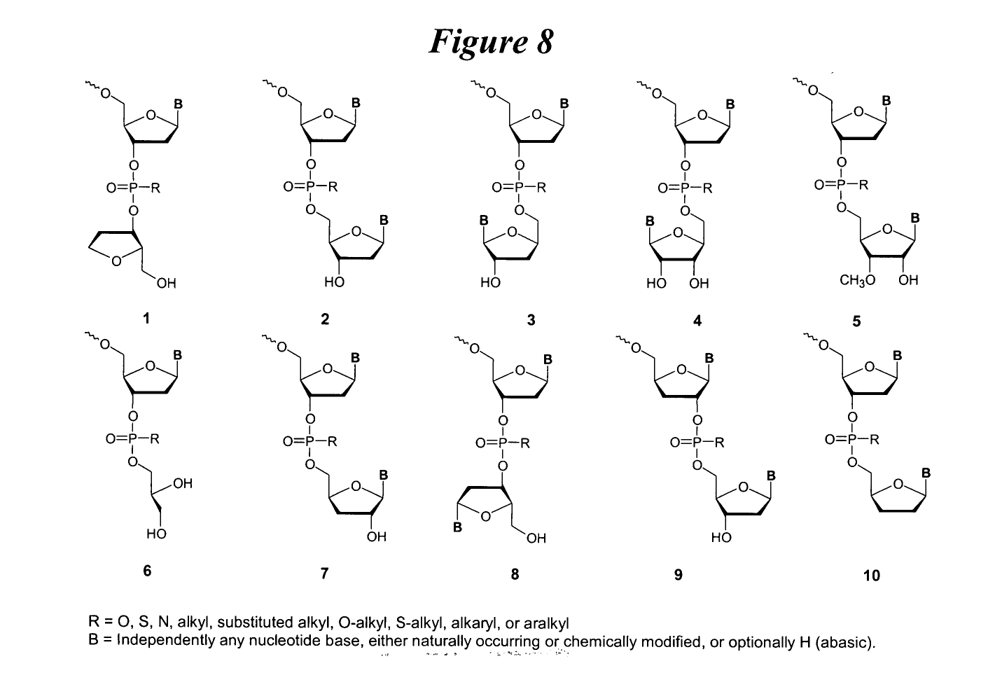

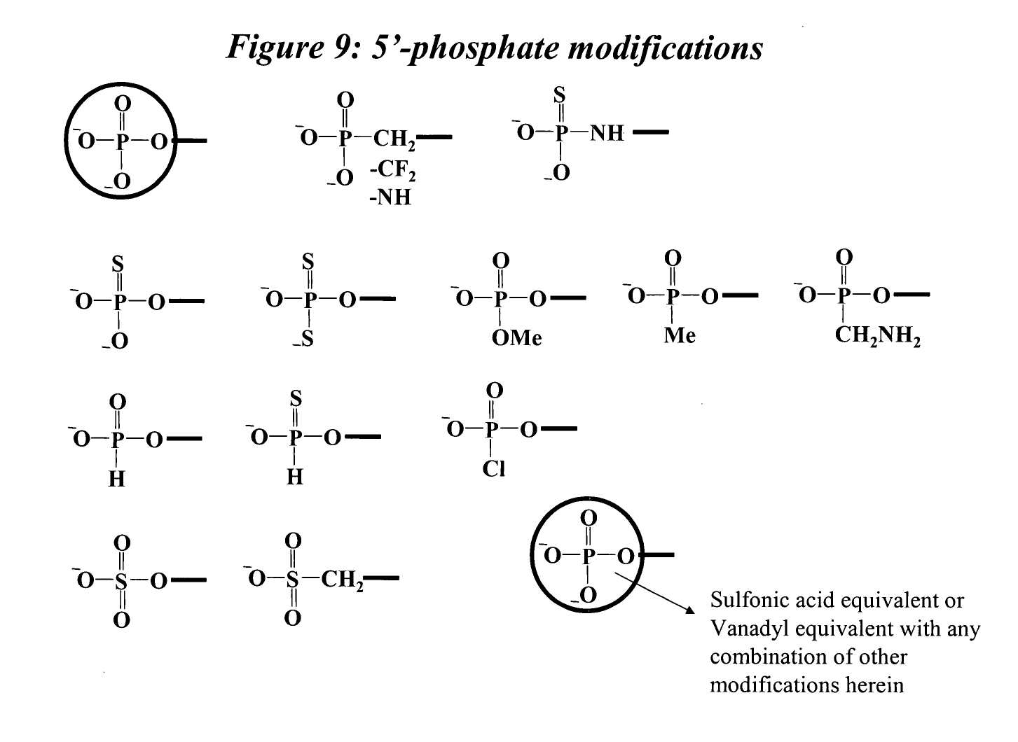

To test the efficacy of DFOs targeted against HBV RNA, DFO duplexes targeting HBV pregenomic RNA were co-transfected with HBV genomic DNA once at 25 nM with lipid at 12.5 μg/ml into Hep G2 cells, and the subsequent levels of secreted HBV surface antigen (HBsAg) were analyzed by ELISA. A DFO construct comprising self complementary sequence (compound # 32221) was assayed with a chemically modified siNA targeting HBV site 1580 (compound # 31335/31337), a corresponding matched chemistry inverted control (compound # 31336/31338), and untreated cells. The self complementary DFO construct was tested both as a preannealed duplex (compound # 32221) or as a single stranded hairpin (compound # 32221 fold), as confirmed by gel electrophoresis, (see FIG. 8). Immulon 4 (Dynax) microtiter wells were coated overnight at 4° C. with anti-HBsAg Mab (Biostride B88-95-31ad,ay) at 1 μg/ml in Carbonate Buffer (Na2CO3 15 mM, NaHCO3 35 mM, pH 9.5). The wells were then washed 4× with PBST (PBS, 0.05% Tweeng 20) and blocked for 1 hr at 37° C. with PBST, 1% BSA. Following washing as above, the wells were dried at 37° C. for 30 min. Biotinylated goat anti-HBsAg (Accurate YVS1807) was diluted 1:1000 in PBST and incubated in the wells for 1 hr. at 37° C. The wells were washed 4× with PBST. Streptavidin/Alkaline Phosphatase Conjugate (Pierce 21324) was diluted to 250 ng/ml in PBST, and incubated in the wells for 1 hr. at 37° C. After washing as above, p-nitrophenyl phosphate substrate (Pierce 37620) was added to the wells, which were then incubated for 1 hour at 37° C. The optical density at 450 nm was then determined. As shown in FIG. 8, the self complementary DFO construct 32221 in duplex form shows significant inhibition of HBsAg.

### Example 9

### Animal Models

Various animal models can be used to screen DFO constructs in vivo as are known in the art, for example those animal models that are used to evaluate other nucleic acid technologies such as enzymatic nucleic acid molecules (ribozymes) and/or antisense. Such animal models are used to test the efficacy of DFO molecules described herein. In a non-limiting example, DFO molecules that are designed as anti-angiogenic agents can be screened using animal models. There are several animal models available in which to test the anti-angiogenesis effect of nucleic acids of the present invention, such as DFO, directed against genes associated with angiogenesis and/or metastais, such as VEGF or VEGFR (e.g., VEGFR1, VEGFR2, and VEGFR3) genes. Typically a corneal model has been used to study angiogenesis in rat and rabbit, since recruitment of vessels can easily be followed in this normally avascular tissue (Pandey et al., 1995 Science 268: 567-569). In these models, a small Teflon or Hydron disk pretreated with an angiogenesis factor (e.g. bFGF or VEGF) is inserted into a pocket surgically created in the cornea. Angiogenesis is monitored 3 to 5 days later. DFO molecules directed against VEGFR mRNAs would be delivered in the disk as well, or dropwise to the eye over the time course of the experiment. In another eye model, hypoxia has been shown to cause both increased expression of VEGF and neovascularization in the retina (Pierce et al., 1995 Proc. Natl. Acad. Sci. USA. 92: 905-909; Shweiki et al., 1992 J. Clin. Invest. 91: 2235-2243).

Several animal models exist for screening of anti-angiogenic agents. These include corneal vessel formation following corneal injury (Burger et al., 1985 Cornea 4: 35-41; Lepri, et al., 1994 J. Ocular Pharmacol. 10: 273-280; Ormerod et al., 1990 Am. J. Pathol. 137: 1243-1252) or intracorneal growth factor implant (Grant et al., 1993 Diabetologia 36: 282-291; Pandey et al. 1995 supra, Zieche et al., 1992 Lab. Invest. 67: 711-715), vessel growth into Matrigel matrix containing growth factors (Passaniti et al., 1992 supra), female reproductive organ neovascularization following hormonal manipulation (Shweiki et al., 1993 Clin. Invest. 91: 2235-2243), several models involving inhibition of tumor growth in highly vascularized solid tumors (O'Reilly et al., 1994 Cell 79: 315-328; Senger et al., 1993 Cancer and Metas. Rev. 12: 303-324; Takahasi et al., 1994 Cancer Res. 54: 4233-4237; Kim et al., 1993 supra), and transient hypoxia-induced neovascularization in the mouse retina (Pierce et al., 1995 Proc. Natl. Acad. Sci. USA. 92: 905-909).

The cornea model, described in Pandey et al. supra, is the most common and well characterized anti-angiogenic agent efficacy screening model. This model involves an avascular tissue into which vessels are recruited by a stimulating agent (growth factor, thermal or alkalai burn, silver nitrate, endotoxin). The corneal model utilizes the intrastromal corneal implantation of a Teflon pellet soaked in a VEGF-Hydron solution to recruit blood vessels toward the pellet, which can be quantitated using standard microscopic and image analysis techniques. To evaluate their anti-angiogenic efficacy, DFO molecules are applied topically to the eye or bound within Hydron on the Teflon pellet itself. This avascular cornea as well as the Matrigel model (described below) provide for low background assays. While the corneal model has been performed extensively in the rabbit, studies in the rat have also been conducted.

The mouse model (Passaniti et al., supra) is a non-tissue model which utilizes Matrigel, an extract of basement membrane (Kleinman et al., 1986) or Millipore® filter disk, which can be impregnated with growth factors and anti-angiogenic agents in a liquid form prior to injection. Upon subcutaneous administration at body temperature, the Matrigel or Millipore® filter disk forms a solid implant. VEGF embedded in the Matrigel or Millipore® filter disk is used to recruit vessels within the matrix of the Matrigel or Millipore® filter disk which can be processed histologically for endothelial cell specific vWF (factor VIII antigen) immunohistochemistry, Trichrome-Masson stain, or hemoglobin content. Like the cornea, the Matrigel or Millipore® filter disk are avascular; however, it is not tissue. In the Matrigel or Millipore® filter disk model, DFO molecules are administered within the matrix of the Matrigel or Millipore® filter disk to test their anti-angiogenic efficacy. Thus, delivery issues in this model, as with delivery of DFO molecules by Hydron-coated Teflon pellets in the rat cornea model, may be less problematic due to the homogeneous presence of the DFO within the respective matrix.

The Lewis lung carcinoma and B-16 murine melanoma models are well accepted models of primary and metastatic cancer and are used for initial screening of anti-cancer agents. These murine models are not dependent upon the use of immunodeficient mice, are relatively inexpensive, and minimize housing concerns. Both the Lewis lung and B-16 melanoma models involve subcutaneous implantation of approximately 10⁶ tumor cells from metastatically aggressive tumor cell lines (Lewis lung lines 3LL or D122, LLc-LN7; B-16-BL6 melanoma) in C57BL/6J mice. Alternatively, the Lewis lung model can be produced by the surgical implantation of tumor spheres (approximately 0.8 mm in diameter). Metastasis also may be modeled by injecting the tumor cells directly intraveneously. In the Lewis lung model, microscopic metastases can be observed approximately 14 days following implantation with quantifiable macroscopic metastatic tumors developing within 21-25 days. The B-16 melanoma exhibits a similar time course with tumor neovascularization beginning 4 days following implantation. Since both primary and metastatic tumors exist in these models after 21-25 days in the same animal, multiple measurements can be taken as indices of efficacy. Primary tumor volume and growth latency as well as the number of micro- and macroscopic metastatic lung foci or number of animals exhibiting metastases can be quantitated. The percent increase in lifespan can also be measured. Thus, these models would provide suitable primary efficacy assays for screening systemically administered DFO molecules and DFO formulations.

In the Lewis lung and B-16 melanoma models, systemic pharmacotherapy with a wide variety of agents usually begins 1-7 days following tumor implantation/inoculation with either continuous or multiple administration regimens. Concurrent pharmacokinetic studies can be performed to determine whether sufficient tissue levels of DFO can be achieved for pharmacodynamic effect to be expected. Furthermore, primary tumors and secondary lung metastases can be removed and subjected to a variety of in vitro studies (i.e. target RNA reduction).

Ohno-Matsui et al., 2002, Am. J. Pathology, 160, 711-719 describe a model of severe proliferative retinopathy and retinal detachment in mice under inducible expression of vascular endothelial growth factor. In this model, expression of a VEGF transgene results in elevated levels of ocular VEGF that is associated with severe proliferative retinopathy and retinal detachment. Furthermore, Mori et al., 2001, J. Cellular Physiology, 188, 253-263, describe a model of laser induced choroidal neovascularization that can be used in conjunction with intravitreous or subretianl injection of DFO molecules of the invention to evaluate the efficacy of DFO treatment of severe proliferative retinopathy and retinal detachment.

In utilizing these models to assess DFO activity, VEGF, VEGFR1, VEGFR2, and/or VEGFR3 protein levels can be measured clinically or experimentally by FACS analysis. VEGFR1, VEGFR2, and/or VEGFR3 encoded mRNA levels can be assessed by Northern analysis, RNase-protection, primer extension analysis and/or quantitative RT-PCR. DFO molecules that block VEGFR1, VEGFR2, and/or VEGFR3 protein encoding mRNAs and therefore result in decreased levels of VEGFR1, VEGFR2, and/or VEGFR3 activity by more than 20% in vitro can be identified using the techniques described herein.

### Example 10

### Indications

The DFO molecules of the invention can be used to treat a variety of diseases and conditions through modulation of gene expression. Using the methods described herein, chemically modified DFO molecules can be designed to modulate the expression of any number of target genes, including but not limited to genes associated with cancer, metabolic diseases, infectious diseases such as viral, bacterial or fungal infections, neurologic diseases, musculoskeletal diseases, diseases of the immune system, diseases associated with signaling pathways and cellular messengers, and diseases associated with transport systems including molecular pumps and channels.

Non-limiting examples of various viral genes that can be targeted using DFO molecules of the invention include Hepatitis C Virus (HCV, for example Genbank Accession Nos: D11168, D50483.1, L38318 and S82227), Hepatitis B Virus (HBV, for example GenBank Accession No. AF100308.1), Human Immunodeficiency Virus type 1 (HIV-1, for example GenBank Accession No. U51188), Human Immunodeficiency Virus type 2 (HIV-2, for example GenBank Accession No. X60667), West Nile Virus (WNV for example GenBank accession No. NC_{—}001563), cytomegalovirus (CMV for example GenBank Accession No. NC_{—}001347), respiratory syncytial virus (RSV for example GenBank Accession No. NC_{—}001781), influenza virus (for example example GenBank Accession No. AF037412, rhinovirus (for example, GenBank accession numbers: D00239, X02316, X01087, L24917, M16248, K02121, X01087), papillomavirus (for example GenBank Accession No. NC_{—}001353), Herpes Simplex Virus (HSV for example GenBank Accession No. NC_{—}001345), and other viruses such as HTLV (for example GenBank Accession No. AJ430458) and SARS (for example GenBank Accession No. NC_{—}004718). Due to the high sequence variability of many viral genomes, selection of DFO molecules for broad therapeutic applications would likely involve the conserved regions of the viral genome. Nonlimiting examples of conserved regions of the viral genomes include but are not limited to 5′-Non Coding Regions (NCR), 3′-Non Coding Regions (NCR) LTR regions and/or internal ribosome entry sites (IRES). DFO molecules designed against conserved regions of various viral genomes will enable efficient inhibition of viral replication in diverse patient populations and may ensure the effectiveness of the DFO molecules against viral quasi species which evolve due to mutations in the non-conserved regions of the viral genome.

Non-limiting examples of human genes that can be targeted using DFO molecules of the invention using methods described herein include any human RNA sequence, for example those commonly referred to by Genbank Accession Number. These RNA sequences can be used to design DFO molecules that inhibit gene expression and therefore abrogate diseases, conditions, or infections associated with expression of those genes. Such non-limiting examples of human genes that can be targeted using DFO molecules of the invention include VEGF (for example GenBank Accession No. NM_{—}003376.3), VEGFr (VEGFRL for example GenBank Accession No. XM_{—}067723, VEGFR2 for example GenBank Accession No. AF063658), HER1, HER2, HER3, and HER4 (for example Genbank Accession Nos: NM_{—}005228, NM_{—}004448, NM_{—}001982, and NM_{—}005235 respectively), telomerase (TERT, for example GenBank Accession No. NM_{—}003219), telomerase RNA (for example GenBank Accession No. U86046), NFkappaB, Rel-A (for example GenBank Accession No. NM_{—}005228), NOGO (for example GenBank Accession No. AB020693), NOGOr (for example GenBank Accession No. XM_{—}015620), RAS (for example GenBank Accession No. NM_{—}004283), RAF (for example GenBank Accession No. XM_{—}033884), CD20 (for example GenBank Accession No. X07203), METAP2 (for example GenBank Accession No. NM 003219), CLCAI (for example GenBank Accession No. NM_{—}001285), phospholamban (for example GenBank Accession No. NM_{—}002667), PTPIB (for example GenBank Accession No. M31724), PCNA (for example GenBank Accession No. NM_{—}002592.1), PKC-alpha (for example GenBank Accession No. NM_{—}002737) and others. The genes described herein are provided as non-limiting examples of genes that can be targeted using DFO molecules of the invention. Additional examples of such genes are described by accession number in Beigelman et al., U.S. Ser. No. 60/363,124, filed Mar. 11, 2002 and incorporated by reference herein in its entirety.

The DFO molecule of the invention can also be used in a variety of agricultural applications involving modulation of endogenous or exogenous gene expression in plants using DFO, including use as insecticidal, antiviral and anti-fungal agents or modulate plant traits such as oil and starch profiles and stress resistance.

### Example 11

### Diagnostic Uses

The DFO molecules of the invention can be used in a variety of diagnostic applications, such as in the identification of molecular targets (e.g., RNA) in a variety of applications, for example, in clinical, industrial, environmental, agricultural and/or research settings. Such diagnostic use of DFO molecules involves utilizing reconstituted RNAi systems, for example, using cellular lysates or partially purified cellular lysates. DFO molecules of this invention can be used as diagnostic tools to examine genetic drift and mutations within diseased cells or to detect the presence of endogenous or exogenous, for example viral, RNA in a cell. The close relationship between DFO activity and the structure of the target RNA allows the detection of mutations in any region of the molecule, which alters the base-pairing and three-dimensional structure of the target RNA. By using multiple DFO molecules described in this invention, one can map nucleotide changes, which are important to RNA structure and function in vitro, as well as in cells and tissues. Cleavage of target RNAs with DFO molecules can be used to inhibit gene expression and define the role of specified gene products in the progression of disease or infection. In this manner, other genetic targets can be defined as important mediators of the disease. These experiments will lead to better treatment of the disease progression by affording the possibility of combination therapies (e.g., multiple DFO molecules targeted to different genes, DFO molecules coupled with known small molecule inhibitors, or intermittent treatment with combinations DFO molecules and/or other chemical or biological molecules). Other in vitro uses of DFO molecules of this invention are well known in the art, and include detection of the presence of mRNAs associated with a disease, infection, or related condition. Such RNA is detected by determining the presence of a cleavage product after treatment with a DFO using standard methodologies, for example, fluorescence resonance emission transfer (FRET).

In a specific example, DFO molecules that cleave only wild-type or mutant forms of the target RNA are used for the assay. The first DFO molecules (i.e., those that cleave only wild-type forms of target RNA) are used to identify wild-type RNA present in the sample and the second DFO molecules (i.e., those that cleave only mutant forms of target RNA) are used to identify mutant RNA in the sample. As reaction controls, synthetic substrates of both wild-type and mutant RNA are cleaved by both DFO molecules to demonstrate the relative DFO efficiencies in the reactions and the absence of cleavage of the “non-targeted” RNA species. The cleavage products from the synthetic substrates also serve to generate size markers for the analysis of wild-type and mutant RNAs in the sample population. Thus, each analysis requires two DFO molecules, two substrates and one unknown sample, which is combined into six reactions. The presence of cleavage products is determined using an RNase protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., disease related or infection related) is adequate to establish risk. If probes of comparable specific activity are used for both transcripts, then a qualitative comparison of RNA levels is adequate and decreases the cost of the initial diagnosis. Higher mutant form to wild-type ratios are correlated with higher risk whether RNA levels are compared qualitatively or quantitatively.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims. The present invention teaches one skilled in the art to test various combinations and/or substitutions of chemical modifications described herein toward generating nucleic acid constructs with improved activity for mediating RNAi activity. Such improved activity can comprise improved stability, improved bioavailability, and/or improved activation of cellular responses mediating RNAi. Therefore, the specific embodiments described herein are not limiting and one skilled in the art can readily appreciate that specific combinations of the modifications described herein can be tested without undue experimentation toward identifying DFO molecules with improved RNAi activity.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE I
Synthetic Sequences

| Compound | | | SEQ |
|---|---|---|---|
| # | Aliases | Sequence | ID# |
| 32802 | HVEGFR1:1247L21 (1229C) v1 5′p | pAAUGCUUUAUCAUAUAUAU GAUAAAGC B | 10 |
| 32809 | HVEGFR1:1247L21 (1229C) v2 5′p | pAAUGCUUUAUCAUAUAU GAUAAAGC B | 11 |
| 32810 | HVEGFR1:1247L21 (1229C) v3 5′p | pAAUGCUUUAUCAUAU GAUAAAGC B | 12 |
| 32811 | HVEGFR1:1247L21 (1229C) v4 5′p | pAAUGCUUUAUCAUAU GAUAAAGCA B | 13 |
| 32812 | HVEGFR1:1247L21 (1229C) v5 5′p | pAAUGCUUUAUCAUAUAU GAUAAAGCAUU B | 14 |
| 32748 | HVEGFR1:346U21 stab07 | B GAAcuGAGuuuAAAAGGcATT B | 15 |
| 32755 | HVEGFR1:364L21 (346C) stab08 | uGccuuuuAAAcucAGuucTsT | 16 |
| 32772 | HVEGFR1:346U21 inv stab07 | B AcGGAAAAuuuGAGucAAGTT B | 17 |
| 32779 | HVEGFR1:364L21 (346C) inv stab08 | cuuGAcucAAAuuuuccGuTsT | 18 |
| 33282 | HBV:2389L21 (2371C) stab08 | GcGAGGGAGuucuucuucuTsT | 19 |
| 32289 | HVEGFR1:364L21 (346C) stab10 | UGCCUUUUAAACUCAGUUCTsT | 20 |
| 32296 | HVEGFR1:346U21 inv stab09 | B ACGGAAAAUUUGAGUCAAGTT B | 21 |
| 32303 | HVEGFR1:364L21 (346C) inv stab10 | CUUGACUCAAAUUUUCCGUTsT | 22 |
| 31270 | HVEGFR1:349U21 stab09 | B CUGAGUUUAAAAGGCACCCTT B | 23 |
| 31273 | HVEGFR1:367L21 (349C) stab10 | GGGUGCCUUUUAAACUCAGTsT | 24 |
| 31276 | HVEGFR1:349U21 stab09 inv | B CCCACGGAAAAUUUGAGUCTT B | 25 |
| 31279 | HVEGFR1:367L21 (349C) stab10 inv | GACUCAAAUUUUCCGUGGGTsT | 26 |
| 31335 | HBV:1580U21 stab09 | B UGUGCACUUCGCUUCACCUTT B | 27 |
| 31337 | HBV:1598L21 (1580C) stab10 | AGGUGAAGCGAAGUGCACATsT | 28 |
| 31336 | HBV:1580U21 inv stab09 | B UCCACUUCGCUUCACGUGUTT B | 29 |
| 31338 | HBV:1598L21 (1580C) inv stab10 | ACACGUGAAGCGAAGUGGATsT | 30 |
| 32221 | HBV:1598L21 (1580C) v4 5′p | pAGGUGAAGCGAAGUGCACA CUUCGCUUCA u B | 31 |
| 34092 | HVEGFR1:373L18 (354C) v4 5′p | pUGCUGGGUGCCUUUUAAA AGGCACCCAGC B | 32 |
| 34093 | HVEGFR1:373L17 (354C) v5 5′p | pGCUGGGUGCCUUUUAAA AGGCACCCAGC B | 33 |
| 34094 | HVEGFR1:373L17 (354C) v6 5′p | pGCUGGGUGCCUUUUAAA AGGCACCCAGCT B | 34 |
| 34095 | HVEGFR1:373L17 (354C) v7 5′p | pGCUGGGUGCCUUUUAAA AGGCACCCAG B | 35 |
| 34096 | HVEGFR1:373L16 (354C) v8 5′p | pCUGGGUGCCUUUUAAA AGGCACCCAG B | 36 |
| 34097 | HVEGFR1:373L16 (354C) v9 5′p | pCUGGGUGCCUUUUAAA AGGCACCCA B | 37 |
| 34098 | HVEGFR1:373L15 (354C) v10 5′p | pUGGGUGCCUUUUAAA AGGCACCCA B | 38 |
| 34099 | HVEGFR1:373L15 (354C) v11 5′p | pUGGGUGCCUUUUAAA AGGCACCCAT B | 39 |
| 34100 | HVEGFR1:373L15 (354C) v12 5′p | pUGGGUGCCUUUUAAA AGGCACCCATT B | 40 |
| 34101 | HVEGFR1:1247L21 (1229C) v14 5′p | pUGCUUUAUCAUAUAUAU GAUAAAGCA B | 41 |
| 34102 | HVEGFR1:1247L21 (1229C) v15 5′p | pUGCUUUAUCAUAUAUAU GAUAAAGC B | 42 |
| 34103 | HVEGFR1:1247L21 (1229C) v16 5′p | pGCUUUAUCAUAUAUAU GAUAAAGC B | 43 |
| 34104 | HVEGFR1:1247L17 (1229C) v5 | AAUGCUUUAUCAUAUAU GAUAAAGCAUU B | 44 |
| 34105 | HVEGFR1:1247L17 (1229C) v7 5′p | pAAUGCUUUAUCAUAUAU GAUAAAGCAUUT B | 45 |
| 34106 | HVEGFR1:1247L17 (1229C) v8 5′p | pAAUGCUUUAUCAUAUAU GAUAAAGCAUUTT B | 46 |
| 34107 | HVEGFR1:1247L17 (1229C) v9 5′p | pAAUGCUUUAUCAUAUAU GAUAAAGCAU B | 47 |
| 34108 | HVEGFR1:1247L16 (1229C) v10 5′p | pAUGCUUUAUCAUAUAU GAUAAAGCAU B | 48 |
| 34109 | HVEGFR1:1247L16 (1229C) v11 5′p | pAUGCUUUAUCAUAUAU GAUAAAGCAUT B | 49 |
| 34110 | HVEGFR1:1247L16 (1229C) v12 5′p | pAUGCUUUAUCAUAUAU GAUAAAGCAUTT B | 50 |
| 34111 | HVEGFR1:1247L16 (1229C) v13 5′p | pAUGCUUUAUCAUAUAU GAUAAAGCA B | 51 |
| 34112 | HVEGFR1:1247L17 (1229C) v14 5′p | pAAUGCUUUAUCAUAUAU CUAUAAGCAUU B | 52 |
| 34113 | HVEGFR1:1247L17 (1229C) v15 5′p | pAAUGCUUUUAGUUAUAU GAUAAAGCAUU B | 53 |
| 34114 | HVEGFR1:1247L17 (1229C) v16 5′p | pAAUCCUUAAUCUUAUUU GAUAAAGCAUU B | 54 |
| 34115 | HVEGFR1:1247L17 (1229C) v17 5′p | pAAuGcuuuAucAuAuAu GAuAAAGcAuu B | 55 |
| 34116 | HVEGFR1:1247L17 (1229C) v18 5′p | pAAuGcuuuAucAuAuAu GAuAAAGcAuu B | 56 |
| 34117 | HBV:197L18 (179C) 5′p | pCGAGCAGGGGUCCUAGGA CCCCUGCUCGB | 57 |
| 34118 | HBV:197L17 (179C) 5′p | pGAGCAGGGGUCCUAGGA CCCCUGCUCB | 58 |
| 34119 | HBV:197L16 (179C) 5′p | pAGCAGGGGUCCUAGGA CCCCUGCUB | 59 |
| 34120 | HBV:197L15 (179C) 5′p | pGCAGGGGUCCUAGGA CCCCUGCB | 60 |
| 34121 | HBV:264L19 (246C) 5′p | pCCACCACGAGUCUAGACUC GUGGUGGB | 61 |
| 34122 | HBV:264L17 (246C) 5′p | pACCACGAGUCUAGACUC GUGGUB | 62 |
| 34123 | HBV:264L16 (246C) 5′p | pCCACGAGUCUAGACUC GUGGB | 63 |
| 34124 | HBV:264L15 (246C) 5′p | pCACGAGUCUAGACUC GUGB | 64 |
| 34125 | HBV:1597L17 (1581C) 5′p | pGGUGAAGCGAAGUGCAC UUCGCUUCACCB | 65 |
| 34126 | HBV:1597L16 (1581C) 5′p | pGUGAAGCGAAGUGCAC UUCGCUUCACB | 66 |
| 34127 | HBV:1597L15 (1581C) 5′p | pUGAAGCGAAGUGCAC UUCGCUUCAB | 67 |
| 34128 | HCVb:100L18 (82C) 5′p | pUCAUACUAACGCCAUGGC GUUAGUAUGAB | 68 |
| 34129 | HCVb:100L17 (82C) 5′p | pCAUACUAACGCCAUGGC GUUAGUAUGB | 69 |
| 34130 | HCVb:100L16 (82C) 5′p | pAUACUAACGCCAUGGC GUUAGUAUB | 70 |
| 34131 | HCVb:100L15 (82C) 5′p | pUACUAACGCCAUGGC GUUAGUAB | 71 |
| 34132 | HCVb:144L19 (126C) 5′p | pACUAUGGCUCUCCCGGGAG AGCCAUAGUB | 72 |
| 34133 | HCVb:144L18 (126C) 5′p | pCUAUGGCUCUCCCGGGAG AGCCAUAGB | 73 |
| 34134 | HCVb:144L17 (126C) 5′p | pUAUGGCUCUCCCGGGAG AGCCAUAB | 74 |
| 34135 | HCVb:144L16 (126C) 5′p | pAUGGCUCUCCCGGGAG AGCCAUB | 75 |
| 34136 | HCVb:144L15 (126C) 5′p | pUGGCUCUCCCGGGAG AGCCAB | 76 |
| 34137 | HCVb:172L17 (155C) 5′p | pCCGGUGUACUCACCGGU GAGUACACCGGB | 77 |
| 34138 | HCVb:172L16 (155C) 5′p | pCGGUGUACUCACCGGU GAGUACACCGB | 78 |
| 34139 | HCVb:172L15 (155C) 5′p | pGGUGUACUCACCGGU GAGUACACCB | 79 |
| 34140 | HCVb:332L17 (315C) 5′p | pCUACGAGACCUCCCGGG AGGUCUCGUAGB | 80 |
| 34141 | HCVb:332L16 (315C) 5′p | pUACGAGACCUCCCGGG AGGUCUCGUAB | 81 |
| 34142 | HCVb:332L15 (315C) 5′p | pACGAGACCUCCCGGG AGGUCUCGUB | 82 |

UPPER CASE = ribonucleotide
UPPER CASE UNDERLINE = 2′-O-methyl nucleotide
Lowercase = 2′-deoxy-2′-fluoro nucleotide
T = thymidine
B = inverted deoxyabasic
S = phosphorothioate internucleotide linkage
A = deoxyadenosine
G = deoxyguanosine
p = phosphate

I. VEGFR1 TARGET AND CORRESPONDING PALINDROME SEQUENCES

| | | | | | | |
|---|---|---|---|---|---|---|
| | | | | Palindrome | | |
| Alias | Target Sequence | SEQ ID | Pos | Length | Palindrome | SEQ ID |

| | | | | | | |
|---|---|---|---|---|---|---|
| hVEGFR1:99U19 | CCCGGGGAAGUGGUUGUCU | 83 | 99 | 6 | CCCGGG | 169 |
| hVEGFR1:156U19 | GCCGGCGGCGGCGAACGAG | 84 | 156 | 6 | GCCGGC | 170 |
| hVEGFR1:189U19 | CGGCCGGGUCGUUGGCCGG | 85 | 189 | 6 | CGGCCG | 171 |
| hVEGFR1:247U19 | ACCAUGGUCAGCUACUGGG | 86 | 247 | 8 | ACCAUGGU | 172 |
| hVEGFR1:284U19 | GCGCGCUGCUCAGCUGUCU | 87 | 284 | 6 | GCGCGC | 173 |
| hVEGFR1:294U19 | CAGCUGUCUGCUUCUCACA | 88 | 294 | 6 | CAGCUG | 174 |
| hVEGFR1:354U19 | UUUAAAAGGCACCCAGCAC | 89 | 354 | 6 | UUUAAA | 175 |
| hVEGFR1:513U19 | CUGCAGUACUUUAACCUUG | 90 | 513 | 6 | CUGCAG | 176 |
| hVEGFR1:564U19 | CAGCUGCAAAUAUCUAGCU | 91 | 564 | 6 | CAGCUG | 177 |
| hVEGFR1:622U19 | UAUAUAUUUAUUAGUGAUA | 92 | 622 | 6 | UAUAUA | 178 |
| hVEGFR1:662U19 | UGUACAGUGAAAUCCCCGA | 93 | 662 | 6 | UGUACA | 179 |
| hVEGFR1:706U19 | GAGCUCGUCAUUCCCUGCC | 94 | 706 | 6 | GAGCUC | 180 |
| hVEGFR1:753U19 | UUUAAAAAAGUUUCCACUU | 95 | 753 | 6 | UUUAAA | 181 |
| hVEGFR1:999U19 | CAAUUGUACUGCUACCACU | 96 | 999 | 6 | CAAUUG | 182 |
| hVEGFR1:1212U19 | UGUUAACACCUCAGUGCAU | 97 | 1212 | 8 | UGUUAACA | 183 |
| hVEGFR1:1228U19 | CAUAUAUAUGAUAAAGCAU | 98 | 1228 | 10 | CAUAUAUAUG | 184 |
| hVEGFR1:1418U19 | UAAUUAUCAAGGACGUAAC | 99 | 1418 | 6 | UAAUUA | 185 |
| hVEGFR1:1492U19 | UUUAAAAACCUCACUGCCA | 100 | 1492 | 6 | UUUAAA | 186 |
| hVEGFR1:1616U19 | CAUAUGGUAUCCCUCAACC | 101 | 1616 | 6 | CAUAUG | 187 |
| hVEGFR1:1804U19 | GCUAGCACCUUGGUUGUGG | 102 | 1804 | 6 | GCUAGC | 188 |
| hVEGFR1:1828U19 | UCUAGAAUUUCUGGAAUCU | 103 | 1828 | 6 | UCUAGA | 189 |
| hVEGFR1:1893U19 | AAGCUUUUAUAUCACAGAU | 104 | 1893 | 6 | AAGCUU | 190 |
| hVEGFR1:1930U19 | GUUAACUUGGAAAAAAUGC | 105 | 1930 | 6 | GUUAAC | 191 |
| hVEGFR1:1984U19 | GUUAACAAGUUCUUAUACA | 106 | 1984 | 6 | GUUAAC | 192 |
| hVEGFR1:2074U19 | AUGGCCAUCACUAAGGAGC | 107 | 2074 | 8 | AUGGCCAU | 193 |
| hVEGFR1:2117U19 | UCAUGAAUGUUUCCCUGCA | 108 | 2117 | 6 | UCAUGA | 194 |
| hVEGFR1:2154U19 | CUGCAGAGCCAGGAAUGUA | 109 | 2154 | 6 | CUGCAG | 195 |
| hVEGFR1:2169U19 | UGUAUACACAGGGGAAGAA | 110 | 2169 | 8 | UGUAUACA | 196 |
| hVEGFR1:2252U19 | GUGAUCACACAGUGGCCAU | 111 | 2252 | 8 | GUGAUCAC | 197 |
| hVEGFR1:2264U19 | UGGCCAUCAGCAGUUCCAC | 112 | 2264 | 6 | UGGCCA | 198 |
| hVEGFR1:2332U19 | UUUAAAAACAACCACAAAA | 113 | 2332 | 6 | UUUAAA | 199 |
| hVEGFR1:2525U19 | UGAUCACUCUAACAUGCAC | 114 | 2525 | 6 | UGAUCA | 200 |
| hVEGFR1:2638U19 | AUUAUAAUGGACCCAGAUG | 115 | 2638 | 8 | AUUAUAAU | 201 |
| hVEGFR1:2717U19 | CCCGGGAGAGACUUAAACU | 116 | 2717 | 6 | CCCGGG | 202 |
| hVEGFR1:2904U19 | UGGCCACCAUCUGAACGUG | 117 | 2904 | 6 | UGGCCA | 203 |
| hVEGFR1:2922U19 | GGUUAACCUGCUGGGAGCC | 118 | 2922 | 8 | GGUUAACC | 204 |
| hVEGFR1:3088U19 | CCAGGCCUGGAACAAGGCA | 119 | 3088 | 10 | CCAGGCCUGG | 205 |
| hVEGFR1:3140U19 | AAAGCUUUGCGAGCUCCGG | 120 | 3140 | 8 | AAAGCUUU | 206 |
| hVEGFR1:3150U19 | GAGCUCCGGCUUUCAGGAA | 121 | 3150 | 6 | GAGCUC | 207 |
| hVEGFR1:3240U19 | AGAUCUGAUUUCUUACAGU | 122 | 3240 | 6 | AGAUCU | 208 |
| hVEGFR1:3266U19 | UGGCCAGAGGCAUGGAGUU | 123 | 3266 | 6 | UGGCCA | 209 |
| hVEGFR1:3380U19 | CCCGGGAUAUUUAUAAGAA | 124 | 3380 | 6 | CCCGGG | 210 |
| hVEGFR1:3390U19 | UUAUAAGAACCCCGAUUAU | 125 | 3390 | 6 | UUAUAA | 211 |
| hVEGFR1:3608U19 | GAGCUCCUGAGUACUCUAC | 126 | 3608 | 6 | GAGCUC | 212 |
| hVEGFR1:3616U19 | GAGUACUCUACUCCUGAAA | 127 | 3616 | 8 | GAGUACUC | 213 |
| hVEGFR1:3732U19 | UGUACAACAGGAUGGUAAA | 128 | 3732 | 6 | UGUACA | 214 |
| hVEGFR1:3905U19 | UCAUGAGCCUGGAAAGAAU | 129 | 3905 | 6 | UCAUGA | 215 |
| hVEGFR1:4013U19 | UGAAGCGCUUCACCUGGAC | 130 | 4013 | 12 | UGAAGCGCUUCA | 216 |
| hVEGFR1:4046U19 | AGGCCUCGCUCAAGAUUGA | 131 | 4046 | 6 | AGGCCU | 217 |
| hVEGFR1:4134U19 | CAGCUGUGGGCACGUCAGC | 132 | 4134 | 6 | CAGCUG | 218 |
| hVEGFR1:4262U19 | UCUAGAGUUUGACACGAAG | 133 | 4262 | 6 | UCUAGA | 219 |
| hVEGFR1:4287U19 | UUCUAGAAGCACAUGUGUA | 134 | 4287 | 8 | UUCUAGAA | 220 |
| hVEGFR1:4296U19 | CACAUGUGUAUUUAUACCC | 135 | 4296 | 8 | CACAUGUG | 221 |
| hVEGFR1:4340U19 | UAUGCAUAUAUAAGUUUAC | 136 | 4340 | 8 | UAUGCAUA | 222 |
| hVEGFR1:4371U19 | CCAUGGGAGCCAGCUGCUU | 137 | 4371 | 6 | CCAUGG | 223 |
| hVEGFR1:4381U19 | CAGCUGCUUUUUGUGAUUU | 138 | 4381 | 6 | CAGCUG | 224 |
| hVEGFR1:4594U19 | UGAUCACCCAAUGCAUCAC | 139 | 4594 | 6 | UGAUCA | 225 |
| hVEGFR1:4604U19 | AUGCAUCACGUACCCCACU | 140 | 4604 | 6 | AUGCAU | 226 |
| hVEGFR1:4632U19 | CUGCAGCCCAAAACCCAGG | 141 | 4632 | 6 | CUGCAG | 227 |
| hVEGFR1:4717U19 | AGGCCUAAGACAUGUGAGG | 142 | 4717 | 6 | AGGCCU | 228 |
| hVEGFR1:4726U19 | ACAUGUGAGGAGGAAAAGG | 143 | 4726 | 6 | ACAUGU | 229 |
| hVEGFR1:4889U19 | GGGCCCAGCCAGGAGCAGA | 144 | 4889 | 6 | GGGCCC | 230 |
| hVEGFR1:4983U19 | AAAUUUUAGACCUUUACCU | 145 | 4983 | 6 | AAAUUU | 231 |
| hVEGFR1:5186U19 | UAUUAAUAUAUAGUCCAGA | 146 | 5186 | 8 | UAUUAAUA | 232 |
| hVEGFR1:5681U19 | GGCGCCUACUCUUCAGGGU | 147 | 5681 | 6 | GGCGCC | 233 |
| hVEGFR1:5893U19 | CUGCAGCCAGUCAGAAGCU | 148 | 5893 | 6 | CUGCAG | 234 |
| hVEGFR1:5991U19 | GAGCUCUAAGUAACCGAAG | 149 | 5991 | 6 | GAGCUC | 235 |
| hVEGFR1:6045U19 | UUUAAAGGCUCUCUGUAUG | 150 | 6045 | 6 | UUUAAA | 236 |
| hVEGFR1:6177U19 | AGAUCUAAAUCCAAACAAA | 151 | 6177 | 6 | AGAUCU | 237 |
| hVEGFR1:6269U19 | CAGCUGGCAAUUUUAUAAA | 152 | 6269 | 6 | CAGCUG | 238 |
| hVEGFR1:6280U19 | UUUAUAAAUCAGGUAACUG | 153 | 6280 | 8 | UUUAUAAA | 239 |
| hVEGFR1:6432U19 | UAAUUAAUUCUUAAUCAUU | 154 | 6432 | 6 | UAAUUA | 240 |
| hVEGFR1:6682U19 | AAUAUUCCAAUCAUUUGCC | 155 | 6682 | 6 | AAUAUU | 241 |
| hVEGFR1:6914U19 | GCUAGCCUCAUUUAAAUUG | 156 | 6914 | 6 | GCUAGC | 242 |
| hVEGFR1:6923U19 | AUUUAAAUUGAUUAAAGGA | 157 | 6923 | 8 | AUUUAAAU | 243 |
| hVEGFR1:7065U19 | UUUAAAGUUACUUUUAUAC | 158 | 7065 | 6 | UUUAAA | 244 |
| hVEGFR1:7093U19 | AUAUAUGCUACAGAUAUAA | 159 | 7093 | 6 | AUAUAU | 245 |
| hVEGFR1:7142U19 | UCAUGAUGAAUGUAUUUUG | 160 | 7142 | 6 | UCAUGA | 246 |
| hVEGFR1:7160U19 | GUAUACCAUCUUCAUAUAA | 161 | 7160 | 6 | GUAUAC | 247 |
| hVEGFR1:7188U19 | AAAUAUUUCUUAAUUGGGA | 162 | 7188 | 8 | AAAUAUUU | 248 |
| hVEGFR1:7271U19 | AAAUUUUUCAAAAUACUAA | 163 | 7271 | 6 | AAAUUU | 249 |
| hVEGFR1:7331U19 | AAAUUUAUCCUUGUUUAGA | 164 | 7331 | 6 | AAAUUU | 250 |
| hVEGFR1:7397U19 | AAAUAUUUUCAAUGGAAAA | 165 | 7397 | 8 | AAAUAUUU | 251 |
| hVEGFR1:7448U19 | UUCGAACCUUUCACUUUUU | 166 | 7448 | 6 | UUCGAA | 252 |
| hVEGFR1:7543U19 | AUAUAUUUGACCAUCACCC | 167 | 7543 | 6 | AUAUAU | 253 |
| hVEGFR1:7622U19 | UAUAUAUUCUCUGCUCUUU | 168 | 7622 | 6 | UAUAUA | 254 |

VEGFR2 Target and Corresponding Palindrome Sequences

| | | | | | | |
|---|---|---|---|---|---|---|
| | | SEQ | | Palindrome | | SEQ |
| Alias | Target Sequence | ID | Pos | Length | Palindrome | ID |

| | | | | | | |
|---|---|---|---|---|---|---|
| hVEGFR2:6U19 | GUCCCGGGACCCCGGGAGA | 255 | 6 | 10 | GUCCCGGGAC | 331 |
| hVEGFR2:16U19 | CCCGGGAGAGCGGUCAGUG | 256 | 16 | 6 | CCCGGG | 332 |
| hVEGFR2:76U19 | GCGCGCCGCAGAAAGUCCG | 257 | 76 | 6 | GCGCGC | 333 |
| hVEGFR2:106U19 | GGAUAUCCUCUCCUACCGG | 258 | 106 | 8 | GGAUAUCC | 334 |
| hVEGFR2:140U19 | CUGCAGCCGCCGGUCGGCG | 259 | 140 | 6 | CUGCAG | 335 |
| hVEGFR2:155U19 | GGCGCCCGGGCUCCCUAGC | 260 | 155 | 6 | GGCGCC | 336 |
| hVEGFR2:159U19 | CCCGGGCUCCCUAGCCCUG | 261 | 159 | 6 | CCCGGG | 337 |
| hVEGFR2:235U19 | UCUAGACAGGCGCUGGGAG | 262 | 235 | 6 | UCUAGA | 338 |
| hVEGFR2:291U19 | CUCGAGGUGCAGGAUGCAG | 263 | 291 | 6 | CUCGAG | 339 |
| hVEGFR2:353U19 | CCCGGGCCGCCUCUGUGGG | 264 | 353 | 6 | CCCGGG | 340 |
| hVEGFR2:667U19 | AGAUCUCCAUUUAUUGCUU | 265 | 667 | 6 | AGAUCU | 341 |
| hVEGFR2:710U19 | UGUACAUUACUGAGAACAA | 266 | 710 | 6 | UGUACA | 342 |
| hVEGFR2:875U19 | UGAUCAGCUAUGCUGGCAU | 267 | 875 | 6 | UGAUCA | 343 |
| hVEGFR2:913U19 | AUUAAUGAUGAAAGUUACC | 268 | 913 | 6 | AUUAAU | 344 |
| hVEGFR2:939U19 | UAUGUACAUAGUUGUCGUU | 269 | 939 | 10 | UAUGUACAUA | 345 |
| hVEGFR2:1024U19 | AAGCUUGUCUUAAAUUGUA | 270 | 1024 | 6 | AAGCUU | 346 |
| hVEGFR2:1039U19 | UGUACAGCAAGAACUGAAC | 271 | 1039 | 6 | UGUACA | 347 |
| hVEGFR2:1094U19 | CUUCGAAGCAUCAGCAUAA | 272 | 1094 | 8 | CUUCGAAG | 348 |
| hVEGFR2:1162U19 | AAAUUUUUGAGCACCUUAA | 273 | 1162 | 6 | AAAUUU | 349 |
| hVEGFR2:1181U19 | CUAUAGAUGGUGUAACCCG | 274 | 1181 | 6 | CUAUAG | 350 |
| hVEGFR2:1214U19 | UGUACACCUGUGCAGCAUC | 275 | 1214 | 6 | UGUACA | 351 |
| hVEGFR2:1633U19 | ACAUGUACGGUCUAUGCCA | 276 | 1633 | 6 | ACAUGU | 352 |
| hVEGFR2:1881U19 | UUUGUACAAAUGUGAAGCG | 277 | 1881 | 10 | UUUGUACAAA | 353 |
| hVEGFR2:1939U19 | CACGUGACCAGGGGUCCUG | 278 | 1939 | 6 | CACGUG | 354 |
| hVEGFR2:1966U19 | UUGCAACCUGACAUGCAGC | 279 | 1966 | 6 | UUGCAA | 355 |
| hVEGFR2:2013U19 | GUGCACUGCAGACAGAUCU | 280 | 2013 | 6 | GUGCAC | 356 |
| hVEGFR2:2018U19 | CUGCAGACAGAUCUACGUU | 281 | 2018 | 6 | CUGCAG | 357 |
| hVEGFR2:2026U19 | AGAUCUACGUUUGAGAACC | 282 | 2026 | 6 | AGAUCU | 358 |
| hVEGFR2:2055U19 | CAAGCUUGGCCCACAGCCU | 283 | 2055 | 8 | CAAGCUUG | 359 |
| hVEGFR2:2109U19 | UUGCAAGAACUUGGAUACU | 284 | 2109 | 6 | UUGCAA | 360 |
| hVEGFR2:2177U19 | UGAUCAUGGAGCUUAAGAA | 285 | 2177 | 6 | UGAUCA | 361 |
| hVEGFR2:2188U19 | CUUAAGAAUGCAUCCUUGC | 286 | 2188 | 6 | CUUAAG | 362 |
| hVEGFR2:2195U19 | AUGCAUCCUUGCAGGACCA | 287 | 2195 | 6 | AUGCAU | 363 |
| hVEGFR2:2404U19 | UUUAAAGAUAAUGAGACCC | 288 | 2404 | 6 | UUUAAA | 364 |
| hVEGFR2:2499U19 | AGGCCUCUACACCUGCCAG | 289 | 2499 | 6 | AGGCCU | 365 |
| hVEGFR2:2518U19 | GCAUGCAGUGUUCUUGGCU | 290 | 2518 | 6 | GCAUGC | 366 |
| hVEGFR2:2720U19 | UGGAUCCAGAUGAACUCCC | 291 | 2720 | 8 | UGGAUCCA | 367 |
| hVEGFR2:2783U19 | GGGAAUUCCCCAGAGACCG | 292 | 2783 | 10 | GGGAAUUCCC | 368 |
| hVEGFR2:2837U19 | UUGGCCAAGUGAUUGAAGC | 293 | 2837 | 8 | UUGGCCAA | 369 |
| hVEGFR2:2942U19 | GAGCUCUCAUGUCUGAACU | 294 | 2942 | 6 | GAGCUC | 370 |
| hVEGFR2:3052U19 | GAAUUCUGCAAAUUUGGAA | 295 | 3052 | 6 | GAAUUC | 371 |
| hVEGFR2:3060U19 | CAAAUUUGGAAACCUGUCC | 296 | 3060 | 8 | CAAAUUUG | 372 |
| hVEGFR2:3213U19 | GAGCUCAGCCAGCUCUGGA | 297 | 3213 | 6 | GAGCUC | 373 |
| hVEGFR2:3282U19 | AGAUCUGUAUAAGGACUUC | 298 | 3282 | 6 | AGAUCU | 374 |
| hVEGFR2:3364U19 | UCGCGAAAGUGUAUCCACA | 299 | 3364 | 6 | UCGCGA | 375 |
| hVEGFR2:3452U19 | CCCGGGAUAUUUAUAAAGA | 300 | 3452 | 6 | CCCGGG | 376 |
| hVEGFR2:3461U19 | UUUAUAAAGAUCCAGAUUA | 301 | 3461 | 8 | UUUAUAAA | 377 |
| hVEGFR2:3544U19 | GUGUACACAAUCCAGAGUG | 302 | 3544 | 8 | GUGUACAC | 378 |
| hVEGFR2:3562U19 | GACGUCUGGUCUUUUGGUG | 303 | 3562 | 6 | GACGUC | 379 |
| hVEGFR2:3593U19 | AAAUAUUUUCCUUAGGUGC | 304 | 3593 | 8 | AAAUAUUU | 380 |
| hVEGFR2:3680U19 | GGGCCCCUGAUUAUACUAC | 305 | 3680 | 6 | GGGCCC | 381 |
| hVEGFR2:3792U19 | CUUGCAAGCUAAUGCUCAG | 306 | 3792 | 8 | CUUGCAAG | 382 |
| hVEGFR2:3840U19 | GAUAUCAGAGACUUUGAGC | 307 | 3840 | 6 | GAUAUC | 383 |
| hVEGFR2:3972U19 | UCUGCAGAACAGUAAGCGA | 308 | 3972 | 8 | UCUGCAGA | 384 |
| hVEGFR2:3995U19 | GCCGGCCUGUGAGUGUAAA | 309 | 3995 | 6 | GCCGGC | 385 |
| hVEGFR2:4024U19 | GAUAUCCCGUUAGAAGAAC | 310 | 4024 | 6 | GAUAUC | 386 |
| hVEGFR2:4222U19 | UCCGGAUAUCACUCCGAUG | 311 | 4222 | 6 | UCCGGA | 387 |
| hVEGFR2:4226U19 | GAUAUCACUCCGAUGACAC | 312 | 4226 | 6 | GAUAUC | 388 |
| hVEGFR2:4281U19 | UUUAAAGCUGAUAGAGAUU | 313 | 4281 | 6 | UUUAAA | 389 |
| hVEGFR2:4309U19 | ACCGGUAGCACAGCCCAGA | 314 | 4309 | 6 | ACCGGU | 390 |
| hVEGFR2:4356U19 | GAGCUCUCCUCCUGUUUAA | 315 | 4356 | 6 | GAGCUC | 391 |
| hVEGFR2:4370U19 | UUUAAAAGGAAGCAUCCAC | 316 | 4370 | 6 | UUUAAA | 392 |
| hVEGFR2:4507U19 | CUGCAGGGAGCCAGUCUUC | 317 | 4507 | 6 | CUGCAG | 393 |
| hVEGFR2:4610U19 | UUUAAAAAGCAUUAUCAUG | 318 | 4610 | 6 | UUUAAA | 394 |
| hVEGFR2:4647U19 | CCAUGGGUUUAGAACAAAG | 319 | 4647 | 6 | CCAUGG | 395 |
| hVEGFR2:4843U19 | CUUAAGUGUGGAAUUCGGA | 320 | 4843 | 6 | CUUAAG | 396 |
| hVEGFR2:4853U19 | GAAUUCGGAUUGAUAGAAA | 321 | 4853 | 6 | GAAUUC | 397 |
| hVEGFR2:4879U19 | UAACGUUACCUUGCUUUGG | 322 | 4879 | 8 | UAACGUUA | 398 |
| hVEGFR2:4900U19 | AGUACUGGAGCCUGCAAAU | 323 | 4900 | 6 | AGUACU | 399 |
| hVEGFR2:4916U19 | AAUGCAUUGUGUUUGCUCU | 324 | 4916 | 8 | AAUGCAUU | 400 |
| hVEGFR2:5504U19 | UUAUAACAUCUAUUGUAUU | 325 | 5504 | 6 | UUAUAA | 401 |
| hVEGFR2:5611U19 | UGGUACCAUAGUGUGAAAU | 326 | 5611 | 8 | UGGUACCA | 402 |
| hVEGFR2:5665U19 | AUAUAUUUAUAGUCUGUUU | 327 | 5665 | 6 | AUAUAU | 403 |
| hVEGFR2:5699U19 | UAAUAUAUUAAAGCCUUAU | 328 | 5699 | 10 | UAAUAUAUUA | 404 |
| hVEGFR2:5714U19 | UUAUAUAUAAUGAACUUUG | 329 | 5714 | 10 | UUAUAUAUAA | 405 |
| hVEGFR2:5791U19 | CAAUUGAUGUCAUUUUAUU | 330 | 5791 | 6 | CAAUUG | 406 |

VEGF Target and Corresponding Palindrome Sequences

| | | | | | | |
|---|---|---|---|---|---|---|
| | | SEQ | | Palindrome | | |
| Alias | Target Sequence | ID | Pos | Length | Palindrome | SEQ ID |

| | | | | | | |
|---|---|---|---|---|---|---|
| VEGF:50U19 | GCUAGCACCAGCGCUCUGU | 407 | 50 | 6 | GCUAGC | 428 |
| VEGF:59U19 | AGCGCUCUGUCGGGAGGCG | 408 | 59 | 6 | AGCGCU | 429 |
| VEGF:91U19 | GACCGGUCAGCGGACUCAC | 409 | 91 | 8 | GACCGGUC | 430 |
| VEGF:188U19 | UUUUAAAACUGUAUUGUUU | 410 | 188 | 8 | UUUUAAAA | 431 |
| VEGF:333U19 | GAGCUCCAGAGAGAAGUCG | 411 | 333 | 6 | GAGCUC | 432 |
| VEGF:379U19 | GCGCGCGGGCGUGCGAGCA | 412 | 379 | 6 | GCGCGC | 433 |
| VEGF:474U19 | GGGAUCCCGCAGCUGACCA | 413 | 474 | 8 | GGGAUCCC | 434 |
| VEGF:483U19 | CAGCUGACCAGUCGCGCUG | 414 | 483 | 6 | CAGCUG | 435 |
| VEGF:551U19 | CCGGCCGGCGGCGGACAGU | 415 | 551 | 8 | CCGGCCGG | 436 |
| VEGF:554U19 | GCCGGCGGCGGACAGUGGA | 416 | 554 | 6 | GCCGGC | 437 |
| VEGF:585U19 | CCGCGGGCAGGGGCCGGAG | 417 | 585 | 6 | CCGCGG | 438 |
| VEGF:705U19 | CCGCGCGGGGGAAGCCGAG | 418 | 705 | 8 | CCGCGCGG | 439 |
| VEGF:745U19 | GCUAGCUCGGGCCGGGAGG | 419 | 745 | 6 | GCUAGC | 440 |
| VEGF:874U19 | UGCGCAGACAGUGCUCCAG | 420 | 874 | 6 | UGCGCA | 441 |
| VEGF:894U19 | CGCGCGCGCUCCCCAGGCC | 421 | 894 | 8 | CGCGCGCG | 442 |
| VEGF:915U19 | GGCCCGGGCCUCGGGCCGG | 422 | 915 | 10 | GGCCCGGGCC | 443 |
| VEGF:955U19 | GGCGCCGAGGAGAGCGGGC | 423 | 955 | 6 | GGCGCC | 444 |
| VEGF:1012U19 | GCCGGCCCCGGUCGGGCCU | 424 | 1012 | 6 | GCCGGC | 445 |
| VEGF:1121U19 | CCAUGGCAGAAGGAGGAGG | 425 | 1121 | 6 | CCAUGG | 446 |
| VEGF:1571U19 | UUGUACAAGAUCCGCAGAC | 426 | 1571 | 8 | UUGUACAA | 447 |
| VEGF:1623U19 | UUGCAAGGCGAGGCAGCUU | 427 | 1623 | 6 | UUGCAA | 448 |

TGFbetaR1 Target and Corresponding Palindrome Sequences

| | | | | | | |
|---|---|---|---|---|---|---|
| | | SEQ | | Palindrome | | |
| Alias | Target Sequence | ID | Pos | Length | Palindrome | SEQ ID |

| | | | | | | |
|---|---|---|---|---|---|---|
| TGFbR1:36U19 | CGGCCGGGCCGGGCCGGGC | 449 | 36 | 6 | CGGCCG | 474 |
| TGFbR1:75U19 | CCAUGGAGGCGGCGGUCGC | 450 | 75 | 6 | CCAUGG | 475 |
| TGFbR1:160U19 | CCCGGGGGCGACGGCGUUA | 451 | 160 | 6 | CCCGGG | 476 |
| TGFbR1:197U19 | UGUACAAAAGACAAUUUUA | 452 | 197 | 6 | UGUACA | 477 |
| TGFbR1:312U19 | CUCGAGAUAGGCCGUUUGU | 453 | 312 | 6 | CUCGAG | 478 |
| TGFbR1:333U19 | GUGCACCCUCUUCAAAAAC | 454 | 333 | 6 | GUGCAC | 479 |
| TGFbR1:390U19 | AUUGCAAUAAAAUAGAACU | 455 | 390 | 8 | AUUGCAAU | 480 |
| TGFbR1:456U19 | CAGCUGUCAUUGCUGGACC | 456 | 456 | 6 | CAGCUG | 481 |
| TGFbR1:675U19 | CAAUUGCGAGAACUAUUGU | 457 | 675 | 6 | CAAUUG | 482 |
| TGFbR1:781U19 | CUCUAGAGAAGAACGUUCG | 458 | 781 | 8 | CUCUAGAG | 483 |
| TGFbR1:791U19 | GAACGUUCGUGGUUCCGUG | 459 | 791 | 8 | GAACGUUC | 484 |
| TGFbR1:841U19 | UCAUGAAAACAUCCUGGGA | 460 | 841 | 6 | UCAUGA | 485 |
| TGFbR1:922U19 | UCAUGAGCAUGGAUCCCUU | 461 | 922 | 6 | UCAUGA | 486 |
| TGFbR1:932U19 | GGAUCCCUUUUUGAUUACU | 462 | 932 | 6 | GGAUCC | 487 |
| TGFbR1:1040U19 | GGUACCCAAGGAAAGCCAG | 463 | 1040 | 6 | GGUACC | 488 |
| TGFbR1:1332U19 | GAAUUCAUGAAGAUUACCA | 464 | 1332 | 6 | GAAUUC | 489 |
| TGFbR1:1335U19 | UUCAUGAAGAUUACCAACU | 465 | 1335 | 8 | UUCAUGAA | 490 |
| TGFbR1:1623U19 | CAGAUCUGCUCCUGGGUUU | 466 | 1623 | 8 | CAGAUCUG | 491 |
| TGFbR1:1781U19 | GUGCACUAUGAACGCUUCU | 467 | 1781 | 6 | GUGCAC | 492 |
| TGFbR1:1854U19 | UUUUUAAAAAGAUGAUUGC | 468 | 1854 | 10 | UUUUUAAAAA | 493 |
| TGFbR1:1953U19 | ACAUGUCUUAUUACUAAAG | 469 | 1953 | 6 | ACAUGU | 494 |
| TGFbR1:2056U19 | CUAUAGUUUUUCAGGAUCU | 470 | 2056 | 6 | CUAUAG | 495 |
| TGFbR1:2087U19 | UUAUAAAACUCUUAUCUUG | 471 | 2087 | 6 | UUAUAA | 496 |
| TGFbR1:2150U19 | CAAUUGUAUUUUGUAUACU | 472 | 2150 | 6 | CAAUUG | 497 |
| TGFbR1:2162U19 | GUAUACUAUUAUUGUUCUU | 473 | 2162 | 6 | GUAUAC | 498 |

Target and Corresponding Palindrome Sequences

| | | | | | | |
|---|---|---|---|---|---|---|
| | | SEQ | | Palindrome | | SEQ |
| Alias | Target Sequence | ID | Pos | Length | Palindrome | ID |

| | | | | | | |
|---|---|---|---|---|---|---|
| HIVth:2654U19 | CAAUGGCCAUUGACAGAAG | 499 | 2654 | 12 | CAAUGGCCAUUG | 634 |
| HIVth:4819U19 | UUUUAAAAGAAAAGGGGGG | 500 | 4819 | 8 | UUUUAAAA | 635 |
| HIVth:9102U19 | UUUUAAAAGAAAAGGGGGG | 501 | 9102 | 8 | UUUUAAAA | 636 |
| HIVth:4413U19 | CCAUGCAUGGACAAGUAGA | 502 | 4413 | 10 | CCAUGCAUGG | 637 |
| HIVth:4089U19 | AUGCAUUAGGAAUCAUUCA | 503 | 4089 | 6 | AUGCAU | 638 |
| HIVth:4929U19 | AAAAUUUUCGGGUUUAUUA | 504 | 4929 | 8 | AAAAUUUU | 639 |
| HIVth:7692U19 | CAAUUGGAGAAGUGAAUUA | 505 | 7692 | 6 | CAAUUG | 640 |
| HIVth:2502U19 | CUAUAGGUACAGUAUUAGU | 506 | 2502 | 6 | CUAUAG | 641 |
| HIVth:2724U19 | AAAUUUCAAAAAUUGGGCC | 507 | 2724 | 6 | AAAUUU | 642 |
| HIVth:554U19 | GCUUAAGCCUCAAUAAAGC | 508 | 554 | 8 | GCUUAAGC | 643 |
| HIVth:9679U19 | GCUUAAGCCUCAAUAAAGC | 509 | 9679 | 8 | GCUUAAGC | 644 |
| HIVth:1280U19 | UUUAAAUGCAUGGGUAAAA | 510 | 1280 | 6 | UUUAAA | 645 |
| HIVth:3015U19 | GAUAUCAGUACAAUGUGCU | 511 | 3015 | 6 | GAUAUC | 646 |
| HIVth:3606U19 | AAAUUUAUCAAGAGCCAUU | 512 | 3606 | 6 | AAAUUU | 647 |
| HIVth:2699U19 | UGUACAGAAAUGGAAAAGG | 513 | 2699 | 6 | UGUACA | 648 |
| HIVth:6542U19 | ACAUGUGGAAAAAUAACAU | 514 | 6542 | 6 | ACAUGU | 649 |
| HIVth:7956U19 | GUUGCAACUCACAGUCUGG | 515 | 7956 | 8 | GUUGCAAC | 650 |
| HIVth:4192U19 | GGUACCAGCACACAAAGGA | 516 | 4192 | 6 | GGUACC | 651 |
| HIVth:4381U19 | CAGCUGUGAUAAAUGUCAG | 517 | 4381 | 6 | CAGCUG | 652 |
| HIVth:2925U19 | AUGCAUAUUUUUCAGUUCC | 518 | 2925 | 6 | AUGCAU | 653 |
| HIVth:569U19 | AAGCUUGCCUUGAGUGCUU | 519 | 569 | 6 | AAGCUU | 654 |
| HIVth:2789U19 | AGUACUAAAUGGAGAAAAU | 520 | 2789 | 6 | AGUACU | 655 |
| HIVth:3752U19 | UUUAAACUACCCAUACAAA | 521 | 3752 | 6 | UUUAAA | 656 |
| HIVth:9694U19 | AAGCUUGCCUUGAGUGCUU | 522 | 9694 | 6 | AAGCUU | 657 |
| HIVth:1544U19 | UACUAGUACCCUUCAGGAA | 523 | 1544 | 8 | UACUAGUA | 658 |
| HIVth:2049U19 | CCUAGGAAAAAGGGCUGUU | 524 | 2049 | 6 | CCUAGG | 659 |
| HIVth:3337U19 | CAGCUGGACUGUCAAUGAC | 525 | 3337 | 6 | CAGCUG | 660 |
| HIVth:1285U19 | AUGCAUGGGUAAAAGUAGU | 526 | 1285 | 6 | AUGCAU | 661 |
| HIVth:3748U19 | UAAAUUUAAACUACCCAUA | 527 | 3748 | 8 | UAAAUUUA | 662 |
| HIVth:510U19 | CAGAUCUGAGCCUGGGAGC | 528 | 510 | 8 | CAGAUCUG | 663 |
| HIVth:2694U19 | AAAUUUGUACAGAAAUGGA | 529 | 2694 | 6 | AAAUUU | 664 |
| HIVth:4460U19 | UGUACACAUUUAGAAGGAA | 530 | 4460 | 6 | UGUACA | 665 |
| HIVth:7708U19 | UUAUAUAAAUAUAAAGUAG | 531 | 7708 | 8 | UUAUAUAA | 666 |
| HIVth:9635U19 | CAGAUCUGAGCCUGGGAGC | 532 | 9635 | 8 | CAGAUCUG | 667 |
| HIVth:3863U19 | UGGUACCAGUUAGAGAAAG | 533 | 3863 | 8 | UGGUACCA | 668 |
| HIVth:2581U19 | AAAUUUUCCCAUUAGUCCU | 534 | 2581 | 6 | AAAUUU | 669 |
| HIVth:4780U19 | UCUUAAGACAGCAGUACAA | 535 | 4780 | 8 | UCUUAAGA | 670 |
| HIVth:7000U19 | UGUACACAUGGAAUUAGGC | 536 | 7000 | 6 | UGUACA | 671 |
| HIVth:2044U19 | GGGCCCCUAGGAAAAAGGG | 537 | 2044 | 6 | GGGCCC | 672 |
| HIVth:2964U19 | AGUAUACUGCAUUUACCAU | 538 | 2964 | 8 | AGUAUACU | 673 |
| HIVth:1825U19 | UUUUAAAAGCAUUGGGACC | 539 | 1825 | 8 | UUUUAAAA | 674 |
| HIVth:3315U19 | CUAUAGUGCUGCCAGAAAA | 540 | 3315 | 6 | CUAUAG | 675 |
| HIVth:2578U19 | UUUAAAUUUUCCCAUUAGU | 541 | 2578 | 6 | UUUAAA | 676 |
| HIVth:4513U19 | AUAUAUAGAAGCAGAAGUU | 542 | 4513 | 6 | AUAUAU | 677 |
| HIVth:8302U19 | UAUAUAAAAAUAUUCAUAA | 543 | 8302 | 6 | UAUAUA | 678 |
| HIVth:1376U19 | UUUAAACACCAUGCUAAAC | 544 | 1376 | 6 | UUUAAA | 679 |
| HIVth:4589U19 | UGGCCAGUAAAAACAAUAC | 545 | 4589 | 6 | UGGCCA | 680 |
| HIVth:2006U19 | CAAUUGUGGCAAAGAAGGG | 546 | 2006 | 6 | CAAUUG | 681 |
| HIVth:2907U19 | CAGUACUGGAUGUGGGUGA | 547 | 2907 | 8 | CAGUACUG | 682 |
| HIVth:6533U19 | AAAAUUUUAACAUGUGGAA | 548 | 6533 | 8 | AAAAUUUU | 683 |
| HIVth:8310U19 | AAUAUUCAUAAUGAUAGUA | 549 | 8310 | 6 | AAUAUU | 684 |
| HIVth:468U19 | AAGCAGCUGCUUUUUGCCU | 550 | 468 | 12 | AAGCAGCUGCUU | 685 |
| HIVth:9051U19 | AGGUACCUUUAAGACCAAU | 551 | 9051 | 8 | AGGUACCU | 686 |
| HIVth:9593U19 | AAGCAGCUGCUUUUUGCCU | 552 | 9593 | 12 | AAGCAGCUGCUU | 687 |
| HIVth:749U19 | GCGCGCACGGCAAGAGGCG | 553 | 749 | 6 | GCGCGC | 688 |
| HIVth:1720U19 | ACCGGUUCUAUAAAACUCU | 554 | 1720 | 6 | ACCGGU | 689 |
| HIVth:3623U19 | UUUAAAAAUCUGAAAACAG | 555 | 3623 | 6 | UUUAAA | 690 |
| HIVth:1750U19 | AAGCUUCACAGGAGGUAAA | 556 | 1750 | 6 | AAGCUU | 691 |
| HIVth:5780U19 | AGAAUUCUGCAACAACUGC | 557 | 5780 | 8 | AGAAUUCU | 692 |
| HIVth:3061U19 | AAUAUUCCAAAGUAGCAUG | 558 | 3061 | 6 | AAUAUU | 693 |
| HIVth:6571U19 | AUGCAUGAGGAUAUAAUCA | 559 | 6571 | 6 | AUGCAU | 694 |
| HIVth:794U19 | AAAAUUUUGACUAGCGGAG | 560 | 794 | 8 | AAAAUUUU | 695 |
| HIVth:1058U19 | AUUAUAUAAUACAGUAGCA | 561 | 1058 | 10 | AUUAUAUAAU | 696 |
| HIVth:7140U19 | AAUUAAUUGUACAAGACCC | 562 | 7140 | 8 | AAUUAAUU | 697 |
| HIVth:9088U19 | AGAUCUUAGCCACUUUUUA | 563 | 9088 | 6 | AGAUCU | 698 |
| HIVth:6867U19 | ACAGGCCUGUCCAAAGGUA | 564 | 6867 | 10 | ACAGGCCUGU | 699 |
| HIVth:8642U19 | AAUAUUGGUGGAAUCUCCU | 565 | 8642 | 6 | AAUAUU | 700 |
| HIVth:525U19 | GAGCUCUCUGGCUAACUAG | 566 | 525 | 6 | GAGCUC | 701 |
| HIVth:9650U19 | GAGCUCUCUGGCUAACUAG | 567 | 9650 | 6 | GAGCUC | 702 |
| HIVth:4261U19 | AGUACUAUUUUUAGAUGGA | 568 | 4261 | 6 | AGUACU | 703 |
| HIVth:109U19 | GAUAUCCACUGACCUUUGG | 569 | 109 | 6 | GAUAUC | 704 |
| HIVth:7535U19 | ACAUGUGGCAGGAAGUAGG | 570 | 7535 | 6 | ACAUGU | 705 |
| HIVth:9234U19 | GAUAUCCACUGACCUUUGG | 571 | 9234 | 6 | GAUAUC | 706 |
| HIVth:716U19 | GAGCUCUCUCGACGCAGGA | 572 | 716 | 6 | GAGCUC | 707 |
| HIVth:7146U19 | UUGUACAAGACCCAACAAC | 573 | 7146 | 8 | UUGUACAA | 708 |
| HIVth:675U19 | GGCGCCCGAACAGGGACUU | 574 | 675 | 6 | GGCGCC | 709 |
| HIVth:7603U19 | AAUAUUACAGGGCUGCUAU | 575 | 7603 | 6 | AAUAUU | 710 |
| HIVth:5822U19 | UGUCGACAUAGCAGAAUAG | 576 | 5822 | 8 | UGUCGACA | 711 |
| HIVth:6194U19 | AAUAUUAAGACAAAGAAAA | 577 | 6194 | 6 | AAUAUU | 712 |
| HIVth:5469U19 | CCUAGGUGUGAAUAUCAAG | 578 | 5469 | 6 | CCUAGG | 713 |
| HIVth:6976U19 | UGUACAAAUGUCAGCACAG | 579 | 6976 | 6 | UGUACA | 714 |
| HIVth:7529U19 | UUAUAAACAUGUGGCAGGA | 580 | 7529 | 6 | UUAUAA | 715 |
| HIVth:6440U19 | CAUAUGAUACAGAGGUACA | 581 | 6440 | 6 | CAUAUG | 716 |
| HIVth:8716U19 | GCUAUAGCAGUAGCUGAGG | 582 | 8716 | 8 | GCUAUAGC | 717 |
| HIVth:5672U19 | CUUAAGAAUGAAGCUGUUA | 583 | 5672 | 6 | CUUAAG | 718 |
| HIVth:7658U19 | AGAUCUUCAGACCUGGAGG | 584 | 7658 | 6 | AGAUCU | 719 |
| HIVth:1225U19 | CUAUAGUGCAGAACAUCCA | 585 | 1225 | 6 | CUAUAG | 720 |
| HIVth:30U19 | GAUAUCCUUGAUCUGUGGA | 586 | 30 | 6 | GAUAUC | 721 |
| HIVth:5534U19 | AUUAAUAACACCAAAAAAG | 587 | 5534 | 6 | AUUAAU | 722 |
| HIVth:9155U19 | GAUAUCCUUGAUCUGUGGA | 588 | 9155 | 6 | GAUAUC | 723 |
| HIVth:1609U19 | UUUAUAAAAGAUGGAUAAU | 589 | 1609 | 8 | UUUAUAAA | 724 |
| HIVth:2134U19 | AGAUCUGGCCUUCCUACAA | 590 | 2134 | 6 | AGAUCU | 725 |
| HIVth:9081U19 | CAGCUGUAGAUCUUAGCCA | 591 | 9081 | 6 | CAGCUG | 726 |
| HIVth:2467U19 | UGAUCAGAUACUCAUAGAA | 592 | 2467 | 6 | UGAUCA | 727 |
| HIVth:4685U19 | GGAAUUCCCUACAAUCCCC | 593 | 4685 | 8 | GGAAUUCC | 728 |
| HIVth:1606U19 | AAAUUUAUAAAAGAUGGAU | 594 | 1606 | 6 | AAAUUU | 729 |
| HIVth:6064U19 | AAGCUUCUCUAUCAAAGCA | 595 | 6064 | 6 | AAGCUU | 730 |
| HIVth:305U19 | UCCGGAGUACUUCAAGAAC | 596 | 305 | 6 | UCCGGA | 731 |
| HIVth:5160U19 | CAUAUGUAUGUUUCAGGGA | 597 | 5160 | 6 | CAUAUG | 732 |
| HIVth:5713U19 | CCAUGGCUUAGGGCAACAU | 598 | 5713 | 6 | CCAUGG | 733 |
| HIVth:9430U19 | UCCGGAGUACUUCAAGAAC | 599 | 9430 | 6 | UCCGGA | 734 |
| HIVth:310U19 | AGUACUUCAAGAACUGCUG | 600 | 310 | 6 | AGUACU | 735 |
| HIVth:6384U19 | GGUACCUGUGUGGAAGGAA | 601 | 6384 | 6 | GGUACC | 736 |
| HIVth:9435U19 | AGUACUUCAAGAACUGCUG | 602 | 9435 | 6 | AGUACU | 737 |
| HIVth:1453U19 | CUGCAGAAUGGGAUAGAGU | 603 | 1453 | 6 | CUGCAG | 738 |
| HIVth:868U19 | AUCGAUGGGAAAAAAUUCG | 604 | 868 | 6 | AUCGAU | 739 |
| HIVth:1123U19 | AAGCUUUAGACAAGAUAGA | 605 | 1123 | 6 | AAGCUU | 740 |
| HIVth:6645U19 | UUUAAAGUGCACUGAUUUG | 606 | 6645 | 6 | UUUAAA | 741 |
| HIVth:1183U19 | CAGCUGACACAGGACACAG | 607 | 1183 | 6 | CAGCUG | 742 |
| HIVth:6091U19 | UACAUGUAAUGCAACCUAU | 608 | 6091 | 8 | UACAUGUA | 743 |
| HIVth:8178U19 | AAGCUUAAUACACUCCUUA | 609 | 8178 | 6 | AAGCUU | 744 |
| HIVth:6650U19 | AGUGCACUGAUUUGAAGAA | 610 | 6650 | 8 | AGUGCACU | 745 |
| HIVth:6779U19 | AUGCAUUUUUUUAUAAACU | 611 | 6779 | 6 | AUGCAU | 746 |
| HIVth:7284U19 | UUUAAAACAGAUAGCUAGC | 612 | 7284 | 6 | UUUAAA | 747 |
| HIVth:7295U19 | UAGCUAGCAAAUUAAGAGA | 613 | 7295 | 6 | UAGCUA | 748 |
| HIVth:8933U19 | UCUCGAGACCUAGAAAAAC | 614 | 8933 | 8 | UCUCGAGA | 749 |
| HIVth:4569U19 | UUUUAAAAUUAGCAGGAAG | 615 | 4569 | 8 | UUUUAAAA | 750 |
| HIVth:6788U19 | UUUAUAAACUUGAUAUAAU | 616 | 6788 | 8 | UUUAUAAA | 751 |
| HIVth:7462U19 | AGUACUGAAGGGUCAAAUA | 617 | 7462 | 6 | AGUACU | 752 |
| HIVth:8512U19 | GGAUCCUUAGCACUUAUCU | 618 | 8512 | 6 | GGAUCC | 753 |
| HIVth:5442U19 | AAGGCCUUAUUAGGACACA | 619 | 5442 | 8 | AAGGCCUU | 754 |
| HIVth:7120U19 | CAGCUGAACACAUCUGUAG | 620 | 7120 | 6 | CAGCUG | 755 |
| HIVth:1872U19 | GCAUGCCAGGGAGUAGGAG | 621 | 1872 | 6 | GCAUGC | 756 |
| HIVth:6037U19 | GAGCUCAUCAGAACAGUCA | 622 | 6037 | 6 | GAGCUC | 757 |
| HIVth:7073U19 | UAAUUAGAUCUGUCAAUUU | 623 | 7073 | 6 | UAAUUA | 758 |
| HIVth:7078U19 | AGAUCUGUCAAUUUCACGG | 624 | 7078 | 6 | AGAUCU | 759 |
| HIVth:5980U19 | UCAUGACAAAAGCCUUAGG | 625 | 5980 | 6 | UCAUGA | 760 |
| HIVth:1480U19 | UGCAUGCAGGGCCUAUCGC | 626 | 1480 | 8 | UGCAUGCA | 761 |
| HIVth:8662U19 | CAAUAUUGGAGUCAGGAGC | 627 | 8662 | 8 | CAAUAUUG | 762 |
| HIVth:292U19 | CUCGAGAGCUGCAUCCGGA | 628 | 292 | 6 | CUCGAG | 763 |
| HIVth:5698U19 | UCCUAGGAUUUAGCUCCAU | 629 | 5698 | 8 | UCCUAGGA | 764 |
| HIVth:9417U19 | CUCGAGAGCUGCAUCCGGA | 630 | 9417 | 6 | CUCGAG | 765 |
| HIVth:4176U19 | AGAUCUAUCUGGCAUGGGU | 631 | 4176 | 6 | AGAUCU | 766 |
| HIVth:7438U19 | AGUACUUGGAGUAAUAGUA | 632 | 7438 | 6 | AGUACU | 767 |
| HIVth:7453U19 | AGUACUUUGAGUACUGAAG | 633 | 7453 | 6 | AGUACU | 768 |

TABLE II

| | | | | | |
|---|---|---|---|---|---|
| Reagent | Equivalents | Amount | Wait Time* DNA | Wait Time* 2′-O-methyl | Wait Time*RNA |

A. 2.5 μmol Synthesis Cycle ABI 394 Instrument

| | | | | | |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl | 186 | 233 μL | 5 sec | 5 sec | 5 sec |
| Imidazole | | | | | |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| | | | | | |
|---|---|---|---|---|---|
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl | 1245 | 124 μL | 5 sec | 5 sec | 5 sec |
| Imidazole | | | | | |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |

C. 0.2 μmol Synthesis Cycle 96 well Instrument

| | Equivalents: DNA/ | Amount: DNA/2′-O- | | | Wait Time* 2′-O- | |
|---|---|---|---|---|---|---|
| Reagent | 2′-O-methyl/Ribo | methyl/Ribo | | Wait Time* DNA | methyl | Wait Time* Ribo |
| Phosphoramidites | 22/33/66 | 40/60/120 | μL | 60 sec | 180 sec | 360 sec |
| S-Ethyl Tetrazole | 70/105/210 | 40/60/120 | μL | 60 sec | 180 min | 360 sec |
| Acetic Anhydride | 265/265/265 | 50/50/50 | μL | 10 sec | 10 sec | 10 sec |
| N-Methyl | 502/502/502 | 50/50/50 | μL | 10 sec | 10 sec | 10 sec |
| Imidazole | | | | | | |
| TCA | 238/475/475 | 250/500/500 | μL | 15 sec | 15 sec | 15 sec |
| Iodine | 6.8/6.8/6.8 | 80/80/80 | μL | 30 sec | 30 sec | 30 sec |
| Beaucage | 34/51/51 | 80/120/120 | | 100 sec | 200 sec | 200 sec |
| Acetonitrile | NA | 1150/1150/1150 | μL | NA | NA | NA |

*Wait time does not include contact time during delivery.
*Tandem synthesis utilizes double coupling of linker molecule

TABLE III
Non-limiting examples of Stabilization Chemistries for chemically
modified DFO constructs

| | | | | Phosphorothioate |
|---|---|---|---|---|
| Chemistry | Pyrimidine | Purine | CAP | linkage |
| “Stab 1” | Ribo | Ribo | — | 5 at 5′-end |
| | | | | 1 at 3′-end |
| “Stab 2” | Ribo | Ribo | — | All linkages |
| “Stab 3” | 2′-fluoro | Ribo | — | 4 at 5′-end |
| | | | | 4 at 3′-end |
| “Stab 4” | 2′-fluoro | Ribo | 5′ and/or | — |
| | | | 3′-ends | |
| “Stab 5” | 2′-fluoro | Ribo | — | 1 at 3′-end |
| “Stab 6” | 2′-O-Methyl | Ribo | 5′ and/or | — |
| | | | 3′-ends | |
| “Stab 7” | 2′-fluoro | 2′-deoxy | 5′ and/or | — |
| | | | 3′-ends | |
| “Stab 8” | 2′-fluoro | 2′-O- | — | 1 at 3′-end |
| | | Methyl | | |
| “Stab 9” | Ribo | Ribo | 5′ and/or | — |
| | | | 3′-ends | |
| “Stab 10” | Ribo | Ribo | — | 1 at 3′-end |
| “Stab 11” | 2′-fluoro | 2′-deoxy | — | 1 at 3′-end |
| Stab 12 | 2′-fluoro | LNA | 5′ and/or | |
| | | | 3′-ends | |
| “Stab 13” | 2′-fluoro | LNA | | 1 at 3′-end |
| “Stab 14” | 2′-fluoro | 2′-deoxy | | 2 at 5′-end |
| | | | | 1 at 3′-end |
| “Stab 15” | 2′-deoxy | 2′-deoxy | | 2 at 5′-end |
| | | | | 1 at 3′-end |
| “Stab 16 | Ribo | 2′-O- | 5′ and/or | |
| | | Methyl | 3′-ends | |
| “Stab 17” | 2′-O-Methyl | 2′-O- | 5′ and/or | |
| | | Methyl | 3′-ends | |
| “Stab 18” | 2′-fluoro | 2′-O- | | 1 at 3′-end |
| | | Methyl | | |

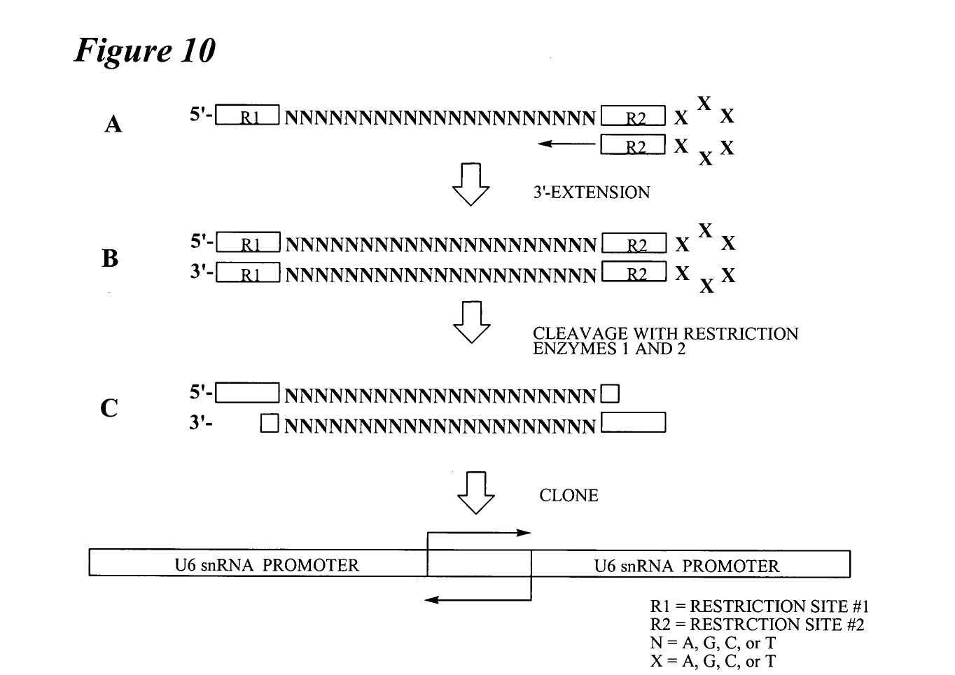

CAP = any terminal cap, see for example FIG. 10.
All Stab 1-18 chemistries can comprise 3′-terminal thymidine (TT) residue

1. A duplex forming oligonucleotide (DFO) comprising a first region having nucleotide sequence complementary to nucleotide sequence of a target RNA sequence or a portion thereof, and a second region having nucleotide sequence that is an inverted repeat of the nucleotide sequence in said first region, wherein said DFO can assemble into a double stranded oligonucleotide, and wherein the nucleotide sequence of each strand of the double stranded oligonucleotide is identical.

2. The DFO molecule of claim 1, wherein said first region and said second region are separated by a palindrome sequence.

3. The DFO molecule of claim 2, wherein said palindrome is about 2 to about 12 nucleotides in length.

4. The DFO molecule of claim 1, wherein said DFO comprises a 3′-terminal cap moiety.

5. The DFO molecule of claim 4, wherein said terminal cap moiety is an inverted deoxyabasic moiety.

6. The DFO molecule of claim 4, wherein said terminal cap moiety is an inverted deoxynucleotide moiety.

7. The DFO molecule of claim 4, wherein said terminal cap moiety is a dinucleotide moiety.

8. The DFO molecule of claim 7, wherein said dinucleotide is dithymidine (TT).

9. The DFO molecule of claim 1, wherein said DFO molecule comprises a 5′-phosphate group.

10. The DFO molecule of claim 1, wherein said DFO molecule comprises no ribonucleotides.

11. The DFO molecule of claim 1, wherein said DFO molecule comprises ribonucleotides.

12. The DFO molecule of claim 1, wherein said DFO comprises at least about 15 nucleotides that are complementary to the nucleotide sequence in said target RNA or a portion thereof.

13. The DFO molecule of claim 1, wherein said DFO comprises at least about 17 nucleotides that are complementary to the nucleotide sequence in said target RNA or a portion thereof.

14. The DFO molecule of claim 1, wherein said DFO comprises at least about 19 nucleotides that are complementary to the nucleotide sequence in said target RNA or a portion thereof.

15. The DFO molecule of claim 1, wherein any purine nucleotide in said DFO is a 2′-O-methylpyrimidine nucleotide.

16. The DFO molecule of claim 1, wherein any purine nucleotide in said DFO is a 2′-deoxy purine nucleotide.

17. The DFO molecule of claim 1, wherein any pyrimidine nucleotide in said DFO is a 2′-deoxy-2′-fluoro pyrimidine nucleotide.

18. The DFO molecule of claim 1, wherein said DFO molecule comprises 3′-nucleotide overhangs.

19. The DFO molecule of claim 18, wherein said 3′-overhangs comprise about 1 to about 4 nucleotides.

20. The DFO molecule of claim 19, wherein said nucleotides comprise deoxynucleotides.

21. The DFO molecule of claim 20, wherein said deoxynucleotides are thymidine nucleotides.

22. A pharmaceutical composition comprising the DFO molecule of claim 1 in an acceptable carrier or diluent.
